SEQUENCE LISTING (30 sequences)

SEQ ID NO: 1 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 163 aa, Helicobacter pylori
DIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 3 - protein, 1199 aa, Artificial Sequence
  Synthetic Construct
CVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPSGR

SEQ ID NO: 4 - protein, 1129 aa, Artificial Sequence
  Synthetic Construct
CVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELD

SEQ ID NO: 5 - DNA, 4 bp, Artificial Sequence
  Synthetic Construct
gsgg

SEQ ID NO: 6 - DNA, 7 bp, Artificial Sequence
  Synthetic Construct
  misc_feature (1)..(1): n is a, c, g, or t
ngtggsg

SEQ ID NO: 7 - DNA, 5 bp, Artificial Sequence
  Synthetic Constuct
ggggs

SEQ ID NO: 8 - protein, 14 aa, SARS-CoV-2
MFVFLVLLPLVSSQ

SEQ ID NO: 9 - protein, 243 aa, Artificial Sequence
  Synthetic Construct
MFVFLVLLPLVSSQRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFHHHHHH

SEQ ID NO: 10 - protein, 1262 aa, Artificial Sequence
  Synthetic Construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPSGRGGGGSRMKQIEDKIEEILSKQYHIENEIARIKKLIGERGGSGGHHHHHH

SEQ ID NO: 11 - protein, 1182 aa, Artificial Sequence
  Synthetic Construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPGRMKQIEDKIEEILSKQYHIENEIARIKKLIGERGGSGGHHHHHH

SEQ ID NO: 12 - protein, 1379 aa, Artificial Sequence
  Synthetic Construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPSGRSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 13 - protein, 1309 aa, Artificial Sequence
  Synthetic Construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 14 - protein, 1202 aa, Artificial Sequence
  Synthetic Construct
CVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPSGR

SEQ ID NO: 15 - protein, 1132 aa, Artificial Sequence
  Synthetic Construct
CVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELD

SEQ ID NO: 16 - protein, 1312 aa, Artificial Sequence
  Synthetic Construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFNDILDKIELIGNENHGLYLADQYVKGIAKSRKS

SEQ ID NO: 17 - protein, 1311 aa, Artificial Sequence
  Synthetic Construct
FVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 18 - protein, 1374 aa, Artificial Sequence
  Synthetic Construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 19 - protein, 1257 aa, Artificial Sequence
  Synthetic Construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGGGGSRMKQIEDKIEEILSKQYHIENEIARIKKLIGERGGSGGHHHHHH

SEQ ID NO: 20 - protein, 1195 aa, Artificial Sequence
  Synthetic Construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDGGGGSRMKQIEDKIEEILSKQYHIENEIARIKKLIGERGGSGGHHHHHH

SEQ ID NO: 21 - protein, 1312 aa, Artificial Sequence
  Synthetic Construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 22 - protein, 1312 aa, Artificial Sequence
  Synthetic Construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSGGDIIKLLNEQVNKEMQSSNLYMSMSSWCYTHSLDGAGLFLFDHAAEEYEHAKKLIIFLNENNVPVQLTSISAPEHNFTGLTQIFQKAYEHEQHISESINNIVDHAIKSKDHATFNFLQWYVAEQHEEEVLFNDILDKIELIGNENHGLYLADQYVKGIAKSRKS

SEQ ID NO: 23 - protein, 1312 aa, Artificial Sequence
  Synthetic Construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSGGDIIKLLNEQVNKEMQSSNLYMSMSSWCYTHSLDGAGLFLFDHAAEEYEHAKKLIIFLNENNVPVQLNSTSAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 24 - protein, 1312 aa, Artificial Sequence
  Synthetic Construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSGGDIIKLLNEQVNKEMQSSNLYMSMSSWCYTHSLDGAGLFLFDHAAEEYEHAKKLIIFLNENNVPVQLTSISAPENKTEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 25 - protein, 1312 aa, Artificial Sequence
  Synthetic Construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNNNTGLYLADQYVKGIAKSRKS

SEQ ID NO: 26 - protein, 1312 aa, Artificial Sequence
  Synthetic Construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 27 - protein, 1309 aa, Artificial Sequence
  Synthetic Construct
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHGSASSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILARLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 28 - protein, 1309 aa, Artificial Sequence
  Synthetic Construct
MFVFLVLLPLVSSQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 29 - protein, 1312 aa, Artificial Sequence
  Synthetic Construct
MFVFLVLLPLVSIQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSCMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 30 - protein, 1312 aa, Artificial Sequence
  Synthetic Construct
MFVFLVLLPLVSSQCVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

(19) United States
(12) Patent Application Publication
(10) Pub. No.: US 2023/0399364 A1
(43) Pub. Date: Dec. 14, 2023

(54) Immunogenic Coronavirus Fusion Proteins and Related Methods

(71) Applicants: The Board of Trustees of the Leland Stanford Junior University, Stanford, CA (US); CHAN ZUCKERBERG BIOHUB, INC., San Francisco, CA (US)

(72) Inventors: Abigail E. Powell, Stanford, CA (US); Payton Anders-Benner Weidenbacher, Stanford, CA (US); Natalia Friedland, Stanford, CA (US); Mrinmoy Sanyal, Stanford, CA (US); Peter S. Kim, Stanford, CA (US)

(73) Assignees: The Board of Trustees of the Leland Stanford Junior University, Stanford, CA (US); CZ Biohub SF, LLC, San Francisco, CA (US)

(21) Appl. No.: 18/043,285

(22) PCT Filed: Aug. 27, 2021
(86) PCT No.: PCT/US2021/047885

§ 371 Date: Feb. 27, 2023

Related U.S. Application Data
(60) Provisional application No. 63/196,837, filed on Jun. 4, 2021.
(60) Provisional application No. 63/130,056, filed on Dec. 23, 2020.
(60) Provisional application No. 63/070,961, filed on Aug. 27, 2020.

(51) Int. Cl. C07K 14/165; C07K 14/79; A61P 37/04; C12N 15/63

(52) U.S. Cl. CPC C07K 14/165; C07K 14/79; A61P 37/04; C12N 15/63; C07K 2319/91

(57) ABSTRACT

Provided are fusion proteins including an amino acid sequence of an ectodomain of Spike protein of a coronavirus, such as SARS-CoV-2, joined to an amino acid sequence of a ferritin subunit polypeptide. Nanoparticles including such fusion proteins, with surface-exposed trimers of the ectodomain of the Spike protein of the coronavirus, are also provided. Also provided are nucleic acids and vectors encoding the fusion proteins, cells containing such nucleic acid and vectors, immunogenic compositions including the fusion proteins, the nanoparticles, or the vectors, as well as corresponding methods and kits.

### BACKGROUND

Coronaviruses (CoV) are a large family of viruses that cause human illness ranging from the common cold to more severe diseases, such as Middle East Respiratory Syndrome (MERS) and Severe Acute Respiratory Syndrome (SARS). Coronaviruses are zoonotic, meaning they can be transmitted between animals and humans. Coronaviruses are large, enveloped, single-stranded RNA viruses having a characteristic crown, or corona, around the virions, due to the surface of the virus particle being covered in well-separated, petal-shaped glycoprotein “spikes,” having a diameter of 80-160 nm, that project from the virions. Spike glycoprotein is a Class I viral fusion protein located on an outer envelope of the virion. Spike protein plays an important role in viral infection by interacting with host cell receptors.

Severe Acute Respiratory Syndrome Coronavirus 2 (SARS-CoV-2) is the strain of coronavirus that causes so-called coronavirus disease 2019 (COVID-19), a respiratory illness. SARS-CoV-2 has spread throughout the world and has already resulted in over 16 million cases of COVID-19 and over 600 thousand deaths. SARS-CoV-2 can enter eukaryotic cells via endosomes or plasma membrane fusion. In both routes, spikes on the virion surface bind to the membrane-bound protein Angiotensin-converting enzyme 2 (ACE2) and mediate attachment to the membrane of and entry into a host cell. SARS-CoV-2 is highly infectious and primarily spreads between people through close contact and via respiratory droplets. Long-term control of SARS-CoV-2 will require an effective vaccine that can be made widely available across the globe.

### SUMMARY

The terms “invention,” “the invention,” “this invention” and “the present invention,” as used in this document, are intended to refer broadly to all of the subject matter of this patent application and the claims below. Statements containing these terms should be understood not to limit the subject matter described herein or to limit the meaning or scope of the patent claims below. Covered embodiments of the invention are defined by the claims, not this summary. This summary is a high-level overview of various aspects of the invention and introduces some of the concepts that are described and illustrated in the present document and the accompanying figures. This summary is not intended to identify key or essential features of the claimed subject matter, nor is it intended to be used in isolation to determine the scope of the claimed subject matter. The subject matter should be understood by reference to appropriate portions of the entire specification, any or all figures and each claim. Some of the exemplary embodiments of the present invention are discussed below.

Included among the embodiments of the present invention and described in the present disclosure are fusion proteins of an artificially modified amino acid sequence of a Spike protein of a coronavirus and an amino acid sequence of a ferritin subunit polypeptide. In some exemplary embodiments, the artificially modified amino acid sequence of the Spike protein is an artificially modified amino acid sequence of an ectodomain of the Spike protein with at least 90% sequence identity to SEQ ID NO:3, SEQ ID NO:4, SEQ ID NO:14, or SEQ ID NO:15. In some exemplary embodiments, the coronavirus is SARS-CoV-2. In some exemplary embodiments, the artificially modified amino acid sequence of the Spike protein of the coronavirus comprises a C-terminal deletion of at least an amino acid sequence of heptad repeat 2 (HR2). In some exemplary embodiments, the artificially modified amino acid sequence of the Spike protein of the coronavirus comprises a mutation eliminating a furin recognition site. In some exemplary embodiments, the artificially modified amino acid sequence of the Spike protein of the coronavirus comprises one or more mutations stabilizing the Spike protein a pre-fusion conformation. The ferritin subunit polypeptide can be Helicobacter pylori ferritin subunit polypeptide. In some exemplary embodiments, the amino acid sequence of the ferritin subunit polypeptide is a sequence with at least 90% sequence identity to SEQ ID NO:2. In some exemplary embodiments, the ferritin subunit polypeptide contains one or more (i.e. at least one) artificial glycosylation sites. In some exemplary embodiments, the artificially modified amino acid sequence of the Spike protein of the coronavirus is joined to the amino acid sequence of the ferritin subunit polypeptide by a linker amino acid sequence. In some exemplary embodiments, the amino acid sequence of the fusion protein is a sequence with at least 90% sequence identity to SEQ ID NO:12, SEQ ID NO:13, SEQ ID NO:16, SEQ ID NO:17, or SEQ ID NO:18, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:33, or SEQ ID NO:34.

Also included among the embodiments of the present invention and described in the present disclosure are nanoparticles comprising oligomers of the fusion proteins according to the embodiments of the present invention. The nanoparticles according to the embodiments of the present invention comprise surface-exposed trimers of an ectodomain of the Spike protein of the coronavirus. In some exemplary embodiments, each nanoparticle comprises eight of the surface-exposed trimers of the ectodomain of the Spike protein of the coronavirus. Also included among the embodiments of the present invention and described in the present disclosure are nucleic acids encoding the fusion protein according to the embodiments of the present invention. The nucleic acids according to the embodiments of the present invention can be DNA or RNA. Also included among the embodiments of the present invention and described in the present disclosure are vectors comprising the nucleic acids according to the embodiments of the present invention. Also included among the embodiments of the present invention and described in the present disclosure are cells comprising the nucleic acids according to the embodiments of the present invention, or the vectors according to the embodiments of the present invention. Also included among the embodiments of the present invention and described in the present disclosure are methods of producing fusion proteins according to the embodiments of the present invention. A method of producing a fusion proteins can comprise the steps of: introducing into a cell a nucleic acid according to the embodiments of the present invention, or a vector according to the embodiments of the present invention; incubating the cell under conditions allowing for expression of the fusion protein; and, isolating the fusion protein. Also included among the embodiments of the present invention and described in the present disclosure are methods of producing nanoparticles according to the embodiments of the present invention. A method of producing a nanoparticle can comprise the steps of: introducing into a cell a nucleic acid according to the embodiments of the present invention, or a vector according to the embodiments of the present invention; incubating the cell under conditions allowing for expression of a fusion protein according to the embodiments of the present invention and self-assembly of the nanoparticle; and, isolating the nanoparticle.

Also included among the embodiments of the present invention and described in the present disclosure are immunogenic compositions comprising the fusion proteins according to the embodiments of the present invention, the nanoparticles according to the embodiments of the present invention, the nucleic acids according to the embodiments of the present invention, or the vectors according to the embodiments of the present invention. In some exemplary embodiments, an immunogenic composition comprises two or more different fusion proteins according to the embodiments of the present invention, two or more different nanoparticles according to the embodiments of the present invention, two or more different nucleic acids according to the embodiments of the present invention, or two or more different vectors according to the embodiments of the present invention. The immunogenic compositions can further comprise one or more adjuvants (i.e. at least one), which may comprise alum. In some exemplary embodiments, the immunogenic compositions are lyophilized. Also included among the embodiments of the present invention and described in the present disclosure are kits comprising an immunogenic composition according to one or more of the embodiments of the present invention, and one or more of: a device for administering the immunogenic composition, and an excipient.

Also included among the embodiments of the present invention and described in the present disclosure are methods of inducing an immune response in a subject, the method comprising the step of administering to the subject an immunogenic composition according to the embodiments of the present invention. In such methods, an immunogenic composition can be administered in an amount capable of eliciting a protective immune response against the coronavirus in the subject. The immune response can comprise production of neutralizing antibodies against the coronavirus in the subject. In methods of inducing an immune response in a subject according to the embodiments of the present invention, the subject can be a human.

### BRIEF DESCRIPTION OF THE DRAWINGS

The present disclosure includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIG. 1A is a schematic illustration of SARS-CoV-2 Spike protein antigen polypeptide constructs according to certain aspects of this disclosure.

FIG. 1B is a schematic illustration of three-dimensional structures of SARS-CoV-2 Spike protein antigen polypeptides according to certain aspects of this disclosure, which are based on the structures of Spike trimers determined by cryogenic electron microscopy (cryo-EM) and the structure of Heliocbacter pylori ferritin nanoparticles determined by X-ray crystallography.

FIG. 2A shows a photographic image of the Western blot illustrating the results of expression and characterization of SARS-CoV-2 Spike protein antigens according to certain aspects of this disclosure.

FIG. 2B shows photographic images of the SDS-PAGE gels illustrating the results of expression and characterization of SARS-CoV-2 Spike protein antigens according to certain aspects of this disclosure.

FIG. 3 shows line plots illustrating the results of analytical scale size-exclusion chromatography coupled with multi-angle light scattering (SEC-MALS) of SARS-CoV-2 Spike protein antigens according to certain aspects of this disclosure.

FIG. 4 shows line plots illustrating the results of binding analysis of SARS-CoV-2 Spike protein antigens to human ACE2, purified SARS-CoV-2 reactive monoclonal antibodies CR3022, CB6, and COVA-2-15, and an Ebola virus reactive monoclonal antibody ADI-15731 (as a negative control) by enzyme-linked immunosorbent assay (ELISA) according to certain aspects of this disclosure.

FIG. 5A shows a representative motion-corrected cryo-EM micrograph and reference-free 2D class averages of SARS-CoV-2 SpikeΔC-ferritin fusion protein nanoparticles according to certain aspects of this disclosure.

FIG. 5B, top panel, shows reconstructed cryo-EM map of SARS-CoV-2 SpikeΔC-ferritin fusion protein nanoparticles in two different views according to certain aspects of this disclosure. The bottom panel shows two different views of the atomic model of SARS-CoV-2 SpikeΔC-ferritin fusion protein docked into the cryo-EM map displayed at lower contour level than the top panel according to certain aspects of this disclosure.

FIG. 6 shows dot plots illustrating the results of ELISA binding analysis of the sera extracted at Day 21 after the initial immunization from the mice immunized with SARS-CoV-2 Spike protein antigens according to certain aspects of this disclosure. The antigens are indicated on the X-axes. The binding of the sera to SARS-CoV-2 RBD protein (left graph) and SARS-CoV-2 Spike protein (right graph) was analyzed. Each point shown on the graphs represents an average log₁₀ EC50 value from two technical replicate ELISA curves from a single animal. Each bar in the graphs represents the mean log₁₀ EC50 value of 10 animals, and the error bars represent the standard deviations. Statistical comparisons were performed using Kruskal-Wallis ANOVA followed by Dunn's multiple comparisons. All p values are represented as following. *=p≤0.05, **=p≤0.01, ***=p≤0.001, ****=p≤0.0001.

FIG. 7 shows dot plots illustrating the neutralization properties of the sera extracted at Day 21 after the initial immunization from the mice immunized with SARS-CoV-2 Spike protein antigens assessed using a luciferase-based SARS-CoV-2 Spike pseudotyped lentiviral assay according to certain aspects of this disclosure. The antigens are indicated on the X-axes. The Y-axis is set at the assay limit of quantitation (1:100 serum dilution), and serum samples with neutralization activity less than that were set at the LOQ. Each point represents the log₁₀ IC50 value from a single animal derived from four replicates. To generate the four replicates, each experiment was performed twice on different days, with duplicate experiments performed on each of the days. This generated four normalized dilution curves that were then compiled to get each IC50 value for each animal. Each point on the graph bars represents the mean value for each group of 10 animals, and the error bars represent the standard deviations. Statistical comparisons were performed using Kruskal-Wallis ANOVA followed by Dunn's multiple comparisons. All p values are represented as following: *=p≤0.05, **=p≤0.01, ***=p≤0.001, ****=p≤0.0001

FIG. 8 shows dot plots illustrating the results of ELISA binding analysis of the sera extracted at Day 28 after the initial immunization from the mice immunized with SARS-CoV-2 Spike protein antigens according to certain aspects of this disclosure. The antigens are indicated on the X-axis. The binding of the sera to SARS-CoV-2 RBD protein (left graph) and SARS-CoV-2 Spike protein (right graph) was analyzed. Each point on the graphs represents an average log₁₀ EC50 value from two technical replicate ELISA curves from a single animal. The bars represents the mean of 10 animals, and the error bars represent the standard deviations. Statistical comparisons were performed using Kruskal-Wallis ANOVA followed by Dunn's multiple comparisons. All p values are represented as following: *=p≤0.05, **=p≤0.01, ***=p 0.001, ****=p 0.0001

FIG. 9 shows dot plots illustrating the neutralization properties of the sera extracted at Day 28 after the initial immunization from the mice immunized with SARS-CoV-2 Spike protein antigens according to certain aspects of this disclosure. The antigens are indicated on the X-axis. The neutralization properties were assessed using a luciferase-based SARS-CoV-2 Spike pseudotyped lentiviral assay. The Y-axis is set at the assay limit of quantitation (1:100 serum dilution), and serum samples with neutralization activity less than that were set at the LOQ. Each point shown in the graph represents log₁₀ IC50 value from a single animal derived from four replicates. To generate the four replicates, each experiment was performed twice on different days), with duplicate experiments performed on each of the days. This generated four normalized dilution curves that were then compiled to get each IC50 value for each animal. The bars represent the mean log₁₀ IC50 for each group of 10 animals, and the error bars represent the standard deviations. Statistical comparisons were performed using Kruskal-Wallis ANOVA followed by Dunn's multiple comparisons. All p values are represented as following. *=p≤0.05, **=p≤0.01, ***=p≤0.001, ****=p≤0.0001

FIG. 10 shows dot plots illustrating the results of ELISA binding analysis of IgG1, IgG2a, and IgG2b subclass responses (as indicated on the X-axis) of the sera extracted from experimental mice immunized with two doses SARS-CoV-2 Spike protein antigens according to certain aspects of this disclosure. The antigens are indicated at the top of each panel. Each point on the graphs represents log₁₀ EC50 value from a single animal; each horizontal bar represents the mean log₁₀ EC50 titer for the group of 10 animals; the error bars represent the standard deviations.

FIG. 11A shows dot plots illustrating the ratio of IgG2a/IgG1 EC50s determined by ELISA binding analysis of the sera extracted from experimental mice immunized with two doses SARS-CoV-2 Spike protein antigens according to certain aspects of this disclosure. The antigens are indicated on the X-axis. Each point on the graphs represents the ratio value from a single animal; each horizontal bar represents the mean ratio for the group of 10 animals; the error bars represent the standard deviations.

FIG. 11B shows dot plots illustrating the ratio of IgG2b/IgG1 EC50s determined by ELISA binding analysis of the sera extracted from experimental mice immunized with two doses SARS-CoV-2 Spike protein antigens according to certain aspects of this disclosure. The antigens are indicated on the X-axis. Each point on the graphs represents the ratio value from a single animal; each horizontal bar represents the mean ratio for the group of 10 animals; the error bars represent the standard deviations.

FIG. 12 shows line plots illustrating the results of binding analysis by ELISA evaluating the levels of IgM in the sera extracted from experimental mice immunized with two doses SARS-CoV-2 Spike protein antigens according to certain aspects of this disclosure. The antigens are indicated at the top of each panel. Each point represents an experimental duplicate from each animal (n=10 mice per group) fit with a dose-response association curve; error bars represent standard deviation for each point.

FIG. 13A shows dot plots illustrating the neutralization properties of the sera extracted from the experimental mice at day 28 after administration of different doses (indicated on the X-axis) of a SARS-CoV-2 Spike protein antigen according to certain aspects of this disclosure. The neutralization properties were assessed using a luciferase-based SARS-CoV-2 Spike pseudotyped lentiviral assay according to certain aspects of this disclosure. The Y-axis is set at the assay limit of quantitation (1:100 serum dilution), and serum samples with neutralization activity less than that were set at the LOQ. Each point represents the log₁₀ IC50 value from a single animal. Each horizontal bar represents the mean value for each group of 10 animals, and the error bars represent the standard deviations.

FIG. 13B shows dot plots illustrating the neutralization properties of the sera extracted from the experimental mice at various time points (indicated on the X-axis) after administration a SARS-CoV-2 Spike protein antigen according to certain aspects of this disclosure. The neutralization properties were assessed using a luciferase-based SARS-CoV-2 Spike pseudotyped lentiviral assay according to certain aspects of this disclosure. The Y-axis is set at the assay limit of quantitation (1:100 serum dilution), and serum samples with neutralization activity less than that were set at the LOQ. Each point represents the log₁₀ IC50 value from a single animal. Each horizontal bar represents the mean value for each group of five animals, and the error bars represent the standard deviations.

FIG. 14 shows dot plots illustrating the neutralization properties of the sera extracted from the experimental mice at various time points after the initial immunization (indicated on the X-axis) with SARS-CoV-2 Spike protein antigens (indicated at the top of each panel) according to certain aspects of this disclosure. The neutralization properties were assessed using a luciferase-based SARS-CoV-2 Spike pseudotyped lentiviral assay according to certain aspects of this disclosure. The Y-axis is set at the assay limit of quantitation (1:100 serum dilution), and serum samples with neutralization activity less than that were set at the LOQ. Each point represents the log₁₀ IC50 value from a single animal. Each horizontal bar represents the mean value for each group of five animals, and the error bars represent the standard deviations.

FIG. 15A shows dot plots illustrating the neutralization properties of the sera extracted from the experimental mice immunized with a single dose of 1 μg or 10 μg (as indicated on the X-axis) of SARS-CoV-2 Spike protein antigen according to certain aspects of this disclosure. The sera was collected at week 3 after the initial immunization. The SARS-CoV-2 Spike protein antigen was adjuvanted with either 500 μg Alhydrogel® and 20 μg CpG, or 10 μg Quil-A® and 10 μg MPLA, as indicated at the top of each panel. The neutralization properties were assessed using a luciferase-based SARS-CoV-2 Spike pseudotyped lentiviral assay according to certain aspects of this disclosure. The Y-axis is set at the assay limit of quantitation (1:100 serum dilution), and serum samples with neutralization activity less than that were set at the LOQ. Each point represents the log₁₀ IC50 value from a single animal. Each horizontal bar represents the mean value for each group of 10 or 20 animals (as shown), and the error bars represent the standard deviations.

FIG. 15B shows dot plots illustrating the neutralization properties of the sera extracted from the experimental mice immunized with one (“day 21”) or two (“day 28”) doses of 1 μg or 10 μg (as indicated on the X-axis) of SARS-CoV-2 Spike protein antigen according to certain aspects of this disclosure. The SARS-CoV-2 Spike protein antigen was adjuvanted with either 500 μg Alhydrogel® and 20 μg CpG, AddaVax™, or 10 μg Quil-A® and 10 μg MPLA (as indicated on the X-axis). The neutralization properties were assessed using a luciferase-based SARS-CoV-2 Spike pseudotyped lentiviral assay according to certain aspects of this disclosure. The Y-axis is set at the assay limit of quantitation (1:100 serum dilution), and serum samples with neutralization activity less than that were set at the LOQ. Each point represents the log₁₀ IC50 value from a single animal. Each horizontal bar represents the mean value for each group of 10 animals, and the error bars represent the standard deviations.

FIG. 16 shows dot plots illustrating the neutralization properties of the sera extracted from the experimental mice immunized with two doses of two SARS-CoV-2 Spike protein antigens (as indicated) according to certain aspects of this disclosure. The SARS-CoV-2 Spike protein antigens administered to the experimental mice were adjuvanted with 10 μg Quil-A® and 10 μg MPLA. The sera were collected at days 21, 28, and 56 (as indicated on the X-axis) after the initial immunization. The neutralization properties were assessed using a luciferase-based SARS-CoV-2 Spike pseudotyped lentiviral assay according to certain aspects of this disclosure. The Y-axis is set at the assay limit of quantitation (1:100 serum dilution), and serum samples with neutralization activity less than that were set at the LOQ. Each point represents the log₁₀ IC50 value from a single animal. Each horizontal bar represents the mean value for each group of five animals, and the error bars represent the standard deviations.

FIG. 17A shows a representative size-exclusion chromatography trace of a SARS-CoV-2 Spike protein antigen according to certain aspects of this disclosure.

FIG. 17B shows a bar graph illustrating a comparison of relative amounts SARS-CoV-2 Spike protein antigens expressed and purified according to certain aspects of this disclosure.

FIG. 18 shows plots generated by bio-layer interferometry (BLI) on the Octet® system (Sartorius, Gottingen, Germany) testing binding of SARS-CoV-2 Spike protein antigens according to certain aspects of this disclosure to conformational monoclonal antibodies and to ACE2 receptor. The monoclonal antibodies and ACE2 receptor fused to Fc fragment were immobilized on the biosensor surface, and the sensors were moved into wells containing SARS CoV-2 Spike protein antigens in solution, then into the wells that did not contain the antigens. Association and dissociation of the SARS CoV-2 Spike protein antigens to the antibodies and ACE2 results in changes in optical interference between light waves that reflect back to the spectrophotometers from an internal surface and from the external interface between sensor and solution. The change of the interference was plotted on the Y-axis and used to indicate the binding and dissociation. The magnitude of the change in the nm shift (plotted on the Y axis) is therefore used a surrogate for binding, where, for similar binding partners, a larger change reflects more binding.

FIG. 19 shows dot plots illustrating the neutralization properties of the sera extracted from the experimental mice immunized with two doses of two SARS-CoV-2 Spike protein antigens (as indicated) according to certain aspects of this disclosure. The SARS-CoV-2 Spike protein antigens administered to the experimental mice were adjuvanted with 500 μg Alum (Alhydrogel®, InvivoGen, San Diego, California) and 20 μg CpG (InvivoGen). The sera were collected at days 21 and 42 (as indicated on the X-axis) after the initial immunization. The neutralization properties were assessed using a luciferase-based SARS-CoV-2 Spike pseudotyped lentiviral assay according to certain aspects of this disclosure. The IC50 values are shown as neutralization titers for different groups at indicated time points. Each point represents the log₁₀ IC50 value from a single animal. The significance of differences between the groups were calculated by student-t test and found not significant (NS), as indicated in the plot. Each horizontal bar represents the mean value for each group of ten animals, and the error bars represent the standard deviations.

FIG. 20 shows UV spectra of lyophilized (“Lyo1,” “Lyo2,” and “Lyo3”) and frozen (“Frozen”) SpikeHexaProAC protein antigen samples according to certain aspects of this disclosure.

FIG. 21 shows, in the left panel, a line plot illustrating the results of scanning fluorimetry analysis of lyophilized (“Lyo”) and frozen (“Frozen”) SpikeHexaProAC protein antigen samples according to certain aspects of this disclosure.

FIG. 22 shows the plots generated by BLI on Octet® system to test binding of SARS-CoV-2 Spike protein antigen from lyophilized (“Lyo1,” “Lyo2,” and “Lyo3”) and frozen (“Frozen”) samples according to certain aspects of this disclosure to conformational monoclonal antibodies and to ACE2 receptor. The monoclonal antibodies and ACE2 receptor fused to Fc fragment were immobilized on the biosensor surface, and the sensors were moved into wells containing either frozen and thawed (“Frozen”) of lyophilized and reconstituted (“Lyo 1”-“Lyo 3”) SARS CoV-2 Spike protein antigens in solution, and then into the wells that did not contain the antigens. Association and dissociation of the SARS CoV-2 Spike protein antigens to the antibodies and ACE2 results in changes in optical interference between light waves that reflect back to the spectrophotometers from an internal surface and from the external interface between sensor and solution. The change of the interference was plotted on the Y-axis and used to indicate the binding and dissociation.

FIG. 23 shows dot plots illustrating the binding of SARS-CoV-2 RBD protein (measured by ELISA as described elsewhere in the present disclosure and indicated as EC50 values on the Y-axis) of the sera extracted from the groups of experimental mice immunized with SARS-CoV-2 Spike protein antigen from lyophilized and frozen samples (as indicated on the X-axis, three groups of mice each) according to certain aspects of this disclosure according to certain aspects of this disclosure. Each point represents the log₁₀ EC50 value from a single animal. The statistical differences in titers were analyzed by student t-test and found not significant (NS), as indicated in the plot.

FIG. 24 shows dot plots illustrating the neutralization properties of the sera extracted from the experimental mice immunized with SARS-CoV-2 Spike protein antigen from lyophilized and frozen samples (as indicated on the X-axis, three groups of mice each) according to certain aspects of this disclosure according to certain aspects of this disclosure. The neutralization properties were assessed using a luciferase-based SARS-CoV-2 Spike pseudotyped lentiviral assay according to certain aspects of this disclosure. The IC50 values are shown as neutralization titers for different groups at indicated time points. Each point represents the log₁₀ IC50 value from a single animal.

FIG. 25 shows the plots generated by BLI on the Octet® system testing binding of lyophilized SARS-CoV-2 Spike protein antigen samples to conformational monoclonal antibody CB6 and to ACE2 receptor. The samples of SARS-CoV-2 Spike protein antigen were lyophilized in 10 mM ammonium bicarbonate pH 7.8 with 1%, 5%, or 10% sucrose (as labeled), and SARS-CoV-2 Spike protein antigen samples frozen in either 10 mM ammonium bicarbonate pH 7.8 with 10% sucrose (“AB frozen”) or in PBS with 10% sucrose (“PBS”). The monoclonal antibodies and ACE2 receptor fused to Fc fragment were immobilized on the biosensor surface, and the sensors were moved into wells containing protein antigens in solution, then into the wells that did not contain the antigens. Association and dissociation of the SARS CoV-2 Spike protein antigens to the antibodies and ACE2 results in changes in optical interference between light waves that reflect back to the spectrophotometers from an internal surface and from the external interface between sensor and solution. The change of the interference was plotted on the Y-axis and used to indicate the binding and dissociation. The magnitude of the change in the nm shift (plotted on the Y axis) is therefore used a surrogate for binding, where, for similar binding partners, a larger change reflects more binding.

FIG. 26 shows plots illustrating the results of size exclusion chromatography—multiple angle light scattering (SEC-MALS) testing the properties of SARS-CoV-2 Spike protein antigen lyophilized in volatile ammonium bicarbonate buffer. The protein was tested directly after reconstitution (“DAY1”) and after being stored at room temperature for 4 days (“DAY 4”).

FIG. 27 is a schematic illustration of the position of the engineered glycosylation site in a SARS-CoV-2 Spike fusion protein nanoparticle according to certain aspects of the present disclosure. Ferritin domains are shown in white. The lysine residue mutated to an asparagine residue in the engineered glycosylation site are shown as black spheres. The glutamic acid residue mutated to a threonine residue in the engineered glycosylation site is shown as grey spheres. The black triangle depicts the 3-fold axis of symmetry.

FIG. 28 shows plots generated by BLI on the Octet® system to test binding of SARS-CoV-2 Spike protein antigens according to certain aspects of this disclosure to conformational monoclonal antibodies and to ACE2 receptor. The monoclonal antibodies and ACE2 receptor fused to Fc fragment were immobilized on the biosensor surface and sensors were moved into wells containing SARS-CoV-2 Spike protein antigens in solution, and then into the wells that did not contain the antigens. Association and dissociation of the SARS-CoV-2 Spike protein antigens to the antibodies and ACE2 results in changes in optical interference between light waves that reflect back to the spectrophotometers from an internal surface and from the external interface between sensor and solution. The change of the interference was plotted on the Y-axis and used to indicate the binding and dissociation. The magnitude of the change in the nm shift (plotted on the Y axis) is therefore used a surrogate for binding, where, for similar binding partners, a larger change reflects more binding. The plot labels are as follows: “Original”—SpikeHexaProAC ferritin; “D614G”-SpikeHexaProAC ferritin D614G; “B.1.1.7”—SpikeHexaProAC ferritin B.1.1.7; “B.1.351”-SpikeHexaProAC ferritin B.1.351; “LA”—SpikeHexaProAC ferritin B.1.429; “P1”-SpikeHexaProAC ferritin P1.

FIG. 29 shows “heat maps” of neutralizing activity (determined using SARS-CoV-2 Spike pseudotyped lentivirus neutralization assay) of SARS-CoV-2 Spike protein antigens against the panel of six pseudoviruses according to certain aspects of this disclosure. SARS-CoV-2 Spike protein antigens are listed on the x-axis of each “heat map,” labeled as follows: “Original”—SpikeHexaProAC ferritin; “D614G”—SpikeHexaProAC ferritin D614G; “B.1.1.7”—SpikeHexaProAC ferritin B.1.1.7; “B.1.351”—SpikeHexaProAC ferritin B.1.351; “LA”—SpikeHexaProAC ferritin B.1.429; “P1”—SpikeHexaProAC ferritin P1. The pseudoviruses tested are plotted on the y-axis of each heat map and are based on SARS-CoV-2 strains Wuhan-1 (denoted as “WT”), D614G, B.1.429, B1.1.7, P1, and B.1.351. Each value of the heat map is a log₁₀IC50 value of the pooled serum from the mice immunized with the same SARS-CoV-2 Spike protein antigen against a specific pseudotyped virus.

FIG. 30A is a bar graph illustrating the testing of the neutralization responses in a group of 5 mice immunized with SpikeHexaProAC ferritin (SEQ ID NO:16) and alum according to certain aspects of this disclosure. The IC50 values are shown as neutralization titers for different groups at indicated time points. Each dot represents a serum sample from an individual mouse. The average IC50 values are indicated below the bars for indicated time points.

FIG. 30B is a bar graph illustrating the testing of the neutralization responses in a group of 5 mice immunized with SpikeHexaProAC ferritin (SEQ ID NO:16) and alum, and boosted 21 days after the initial immunization according to certain aspects of this disclosure. The IC50 values are shown as neutralization titers for different groups at indicated time points. Each dot represents a serum sample from an individual mouse. The average IC50 values are indicated below the bars for indicated time points.

FIG. 31A is a bar graph illustrating the testing of the neutralization responses against wild type SARS-CoV-2 and SARS-CoV-2 variants (as indicated above the bars) in groups of 10 mice immunized with SpikeHexaProAC ferritin (SEQ ID NO:16) adjuvanted with alum according to certain aspects of this disclosure. The IC50 values are shown as neutralization titers for different groups. Each dot represents a serum sample from an individual mouse. The average IC50 values are indicated below the bars for indicated time points.

FIG. 31B is a bar graph illustrating the testing of the neutralization responses against wild type SARS-CoV-2 and SARS-CoV-2 variants (as indicated above the bars) in groups of 10 mice immunized with SpikeHexaProAC ferritin (SEQ ID NO:16) adjuvanted with alum, and boosted 21 days after the initial immunization according to certain aspects of this disclosure. The IC50 values are shown as neutralization titers for different groups. Each dot represents a serum sample from an individual mouse. The average IC50 values are indicated below the bars for indicated time points.

FIG. 32A is a bar graph illustrating the testing of the neutralization responses against wild type SARS-CoV-2 and SARS-CoV-2 variants (as indicated above the bars) in groups of 10 mice immunized with SpikeHexaProAC ferritin (SEQ ID NO:16) adjuvanted with alum and CpG according to certain aspects of this disclosure. The IC50 values are shown as neutralization titers for different groups. Each dot represents a serum sample from an individual mouse. The average IC50 values are indicated below the bars.

FIG. 32B is a bar graph illustrating the testing of the neutralization responses against wild type SARS-CoV-2 and SARS-CoV-2 variants (as indicated above the bars) in groups of 10 mice immunized with SpikeHexaProAC ferritin (SEQ ID NO:16) adjuvanted with alum and CpG and boosted 21 days after the initial immunization according to certain aspects of this disclosure. The IC50 values are shown as neutralization titers for different groups. Each dot represents a serum sample from an individual mouse. The average IC50 values are indicated below the bars.

FIG. 33 is a bar graph illustrating the testing of the neutralization responses against wild type SARS-CoV-2 and SARS-CoV-2 variants (as indicated above the bars) in groups of 5 mice immunized with SpikeHexaProAC ferritin (SEQ ID NO:16) adjuvanted with different doses of alum, which are indicated on the x-axis, according to certain aspects of this disclosure. For each alum dose one group received single immunization, and a second group was boosted 21 days after the primary immunization, as indicated above the plot. The IC50 values are shown as neutralization titers for different groups. Each dot represents a serum sample from an individual mouse. The average IC50 values are shown as neutralization titers for different groups at indicated time points.

FIG. 34A is a bar graph illustrating the testing of the neutralization responses against wild type SARS-CoV-2 in groups of 5 mice immunized with SpikeHexaProAC ferritin (SEQ ID NO:16) adjuvanted with different amounts of alum (indicated on the x-axis) either alone or in combination with 20 μg of CpG (as indicated below the x-axis) according to certain aspects of this disclosure. For each alum dose one group received single immunization, and a second group was boosted 21 days after the primary immunization (as indicated below the x-axis). The IC50 values are shown as neutralization titers for different groups. Each dot represents a serum sample from an individual mouse. The average IC50 values are shown as neutralization titers for different groups of pooled samples for each of the indicated groups and time points.

FIG. 34B is a bar graph illustrating the testing of the neutralization responses against B.1.421 variant of SARS-CoV-2 in groups of 5 mice immunized with SpikeHexaProAC ferritin (SEQ ID NO:16) adjuvanted with different amounts of alum (indicated on the x-axis) either alone or in combination with 20 μg of CpG (as indicated below the x-axis) according to certain aspects of this disclosure. For each alum dose one group received single immunization, and a second group was boosted 21 days after the primary immunization (as indicated below the x-axis). The IC50 values are shown as neutralization titers for different groups. Each dot represents a serum sample from an individual mouse. The average IC50 values are shown as neutralization titers for different groups of pooled samples for each of the indicated groups and time points.

FIG. 34C is a bar graph illustrating the testing of the neutralization responses against B.1.1.7. variant of SARS-CoV-2 in groups of 5 mice immunized with SpikeHexaProAC ferritin (SEQ ID NO:16) adjuvanted with different amounts of alum (indicated on the x-axis) either alone or in combination with 20 μg of CpG (as indicated below the x-axis) according to certain aspects of this disclosure. For each alum dose one group received single immunization, and a second group was boosted 21 days after the primary immunization (as indicated below the x-axis). The IC50 values are shown as neutralization titers for different groups. Each dot represents a serum sample from an individual mouse. The average IC50 values are shown as neutralization titers for different groups of pooled samples for each of the indicated groups and time points.

FIG. 34D is a bar graph illustrating the testing of the neutralization responses against B.1.351 variant of SARS-CoV-2 in groups of 5 mice immunized with SpikeHexaProAC ferritin (SEQ ID NO:16) adjuvanted with different amounts of alum (indicated on the x-axis) either alone or in combination with 20 μg of CpG (as indicated below the x-axis) according to certain aspects of this disclosure. For each alum dose one group received single immunization, and a second group was boosted 21 days after the primary immunization (as indicated below the x-axis). The IC50 values are shown as neutralization titers for different groups. Each dot represents a serum sample from an individual mouse. The average IC50 values are shown as neutralization titers for different groups of pooled samples for each of the indicated groups and time points.

FIG. 34E is a bar graph illustrating the testing of the neutralization responses against B.1.617.2 variant of SARS-CoV-2 in groups of 5 mice immunized with SpikeHexaProAC ferritin (SEQ ID NO:16) adjuvanted with different amounts of alum (indicated on the x-axis) either alone or in combination with 20 μg of CpG (as indicated below the x-axis) according to certain aspects of this disclosure. For each alum dose one group received single immunization, and a second group was boosted 21 days after the primary immunization (as indicated below the x-axis). The IC50 values are shown as neutralization titers for different groups. Each dot represents a serum sample from an individual mouse. The average IC50 values are shown as neutralization titers for different groups of pooled samples for each of the indicated groups and time points.

FIG. 34F is a bar graph illustrating the testing of the neutralization responses against P.1 variant of SARS-CoV-2 in groups of 5 mice immunized with SpikeHexaProAC ferritin (SEQ ID NO:16) adjuvanted with different amounts of alum (indicated on the x-axis) either alone or in combination with 20 μg of CpG (as indicated below the x-axis) according to certain aspects of this disclosure. For each alum dose one group received single immunization, and a second group was boosted 21 days after the primary immunization (as indicated below the x-axis). The IC50 values are shown as neutralization titers for different groups. Each dot represents a serum sample from an individual mouse. The average IC50 values are shown as neutralization titers for different groups of pooled samples for each of the indicated groups and time points.

### DETAILED DESCRIPTION

The inventors designed, generated, and characterized fusion proteins of SARS-CoV-2 Spike ectodomain polypeptide and ferritin (“SARS-CoV-2 Spike-ferritin fusion proteins”) that self-assemble into nanoparticles displaying on their surfaces the respective versions of SARS-CoV-2 Spike ectodomain. Some versions of SARS-CoV-2 Spike-ferritin fusion protein contain the full-length ectodomain of SARS-CoV-2 Spike protein. Other versions contain a SARS-CoV-2 Spike protein ectodomain having C-terminal deletions (in one example, a C-terminal deletion of 70 amino acids). The inventors discovered that, surprisingly, a C-terminal deletion in the SARS-CoV-2 Spike protein amino acid sequence considerably improved the expression of the resulting fusion protein in mammalian cells. The inventors confirmed proper folding of Spike domains in each version of SARS-CoV-2 Spike-ferritin fusion proteins into a native-like conformation on the surface of the nanoparticles by cryo-EM, size-exclusion chromatography multi-angle light scattering (SEC-MALS), and bio-layer interferometry (BLI), which measured binding SARS-CoV-2 Spike-ferritin fusion proteins to ACE2 receptor and/or one or more Spike-specific monoclonal antibodies. The inventors tested the immunogenicity of SARS-CoV-2 Spike-ferritin fusion proteins in experimental animals, including comparatively with other SARS-CoV-2 fusion protein antigens. Following a single immunization of mice with SARS-CoV-2 Spike-ferritin fusion proteins, the inventors observed neutralizing antibody amounts comparable to or greater than those seen in human convalescent plasma, as determined using a lentiviral CoV-2 pseudovirus assay. In contrast, a single immunization with either the CoV-2 receptor binding domain (RBD) or isolated Spike trimers of SARS-CoV-2 Spike elicited much weaker neutralizing antibody responses. The inventors also tested SARS-CoV-2 virus neutralizing properties of the antibodies generated in the experimental animals to SARS-CoV-2 Spike-ferritin fusion proteins that were used as immunogens. The inventors discovered that, unexpectedly, SARS-CoV-2 Spike-ferritin fusion proteins capable of self-assembly into nanoparticles elicited significantly stronger antigen-specific and neutralizing antibody responses in the experimental animals as compared to other SARS-CoV-2 Spike protein antigens. The inventors further discovered that the SARS-CoV-2 Spike-ferritin fusion proteins having C-terminal deletion in SARS-CoV-2 Spike protein ectodomain amino acid sequence (“C-terminal deletion”) elicited the highest neutralizing antibody response in the experimental animals among all the antigens tested. The inventors realized that, given the ability of SARS-CoV-2 Spike-ferritin fusion proteins to self-assemble into nanoparticles after production in mammalian cells, the achieved expression levels comparable to those of ectodomain of SARS-CoV-2 Spike protein, and the enhanced immune response elicited by SARS-CoV-2 Spike-ferritin fusion proteins, SARS-CoV-2 Spike-ferritin fusion proteins (including Spike-ferritin fusion proteins having the C-terminal deletion in SARS-CoV-2 Spike protein ectodomain amino acid sequence) can be used in subunit or nucleic acid vaccines against SARS-CoV-2.

The inventors tested several SARS-CoV-2 Spike-ferritin fusion proteins with the C-terminal deletion and two or more proline substitutions and discovered that SARS-CoV-2 Spike-ferritin fusion proteins with the C-terminal deletion and six proline substitutions was equally immunogenic to SARS-CoV-2 Spike-ferritin fusion proteins with the C-terminal deletion and two proline substitutions. Furthermore, expression and purification yields of SARS-CoV-2 Spike-ferritin fusion proteins with the C-terminal deletion and six proline substitutions were unexpectedly and remarkably higher than those for SARS-CoV-2 Spike-ferritin fusion proteins with the C-terminal deletion and fewer proline substitutions. The inventors created and tested several versions of SARS-CoV-2 Spike-ferritin fusion proteins with the C-terminal deletion and six proline substitutions. These versions were based on of naturally occurring variants of coronavirus Spike protein and, when administered to experimental animals, elicited antibodies with high neutralizing activity. The inventors found that lyophilized and subsequently reconstituted SARS-CoV-2 Spike-ferritin fusion proteins retained their structure and immunogenicity. Furthermore, the inventors engineered SARS-CoV-2 Spike ferritin fusion protein antigens with artificial glycosylation sites in the ferritin domain, in order to shield the ferritin domain from the immune system and decrease immune response against the ferritin domain (thus minimizing non-productive immune responses against the anti-SARS-CoV-2 vaccines conceived by the inventors).

Based on the above discoveries, the inventors conceived, and the present disclosure describes, various embodiments of coronavirus Spike-ferritin fusion proteins, nanoparticles composed of such fusion proteins, nucleic acids, nucleic acid constructs and vectors encoding coronavirus Spike-ferritin fusion proteins, as well as cells, compositions, kits, and methods related to production and use of coronavirus Spike-ferritin fusion proteins. The production of nanoparticles of coronavirus Spike-ferritin fusion proteins requires only a single expression plasmid. Expression and purification of coronavirus Spike-ferritin fusion proteins can be carried out and scaled using standard protocols for soluble proteins, with the purified fusion proteins self-assembling into homogenous populations of nanoparticles. In contrast, nanoparticles assembled from separate components require for the components to be generated separately and conjugated in a post purification conjugation step, which can drastically decrease the yield and create heterogeneous nanoparticle populations. Coronavirus Spike-ferritin fusion proteins and the related nucleic acids, nucleic acid constructs, vectors, cells, compositions, kits and methods conceived by the inventors and described in the present disclosure are useful for a variety of application, including, but not limited to, development and production of immunogenic compositions (vaccines), based on proteins or nucleic acids and useful for inducing an immune response against coronavirus infections, as well as for prevention or treatment of coronavirus infections, including, but not limited to, SARS-CoV-2 infection. The experimental results obtained by the inventors demonstrated that that nanoparticles of Spike-ferritin fusion proteins displaying coronavirus Spike protein ectodomain can reliably elicit clinically relevant amounts of neutralizing antibodies in subjects. Accordingly, coronavirus Spike-ferritin fusion proteins and nucleic constructs encoding such fusion proteins can be used as vaccines, such as single-dose vaccines, for inducing protection against coronavirus infection.

### Terms and Concepts

A number of terms and concepts are discussed below. They are intended to facilitate the understanding of various embodiments of the invention in conjunction with the rest of the present document and the accompanying figures. These terms and concepts may be further clarified and understood based on the accepted conventions in the fields of the present invention, as well as the description provided throughout the present document and/or the accompanying figures. Some other terms can be explicitly or implicitly defined in other sections of this document and in the accompanying figures, and may be used and understood based on the accepted conventions in the fields of the present invention, the description provided throughout the present document and/or the accompanying figures. The terms not explicitly defined can also be defined and understood based on the accepted conventions in the fields of the present invention and interpreted in the context of the present document and/or the accompanying figures.

Unless otherwise dictated by context, singular terms shall include pluralities, and plural terms shall include the singular. Generally, nomenclatures used in connection with, and techniques of, cell and tissue culture, molecular biology, immunology, microbiology, genetics and protein and nucleic acid chemistry are those well-known and commonly used. Known methods and techniques are generally performed according to conventional methods well-known and as described in various general and more specific references, unless otherwise indicated. The nomenclatures used in connection with the laboratory procedures and techniques described in the present disclosure are those well-known and commonly used.

As used herein, the terms “a”, “an”, and “the” can refer to one or more unless specifically noted otherwise.

The use of the term “or” is used to mean “and/or,” unless explicitly indicated to refer to alternatives only, or the alternatives are mutually exclusive, although the disclosure supports a definition that refers to only alternatives and “and/or.” As used herein “another” can mean at least a second or more.

The terms “about” and “approximately” as used herein shall generally mean an acceptable degree of error for the quantity measured given the nature or precision of the measurements. Exemplary degrees of error are within 20% (%); preferably, within 10%; and more preferably, within 5% of a given value or range of values. Any reference to “about X” or “approximately X” specifically indicates at least the values X, 0.95X, 0.96X, 0.97X, 0.98X, 0.99X, 1.01X, 1.02X, 1.03X, 1.04X, and 1.05X. Thus, expressions “about X” or “approximately X” are intended to teach and provide written support for a claim limitation of, for example, “0.98X.” Alternatively, in biological systems, the terms “about” and “approximately” may mean values that are within an order of magnitude, preferably within 5-fold, and more preferably within 2-fold of a given value. Numerical quantities given herein are approximate unless stated otherwise, meaning that the term “about” or “approximately” can be inferred when not expressly stated. When “about” is applied to the beginning of a numerical range, it applies to both ends of the range.

The terms “protein,” “peptide,” and “polypeptide” are used interchangeably to refer to a polymer of amino acid residues. The term apply to naturally occurring amino acid polymers and non-natural amino acid polymers, as well as to amino acid polymers in which one (or more) amino acid residue is an artificial chemical mimetic of a corresponding naturally occurring amino acid. The terms encompass amino acid chains of any length, including full-length proteins, wherein the amino acid residues are linked by covalent peptide bonds.

An “isolated” or “purified” polypeptide or protein, or biologically active portion a polypeptide or a protein, is substantially or essentially free from components that normally accompany or interact with the polypeptide or protein as found in its naturally occurring environment. Thus, an isolated or purified polypeptide or protein is substantially free of other cellular material, or culture medium when produced by recombinant techniques, or substantially free of chemical precursors or other chemicals when chemically synthesized. A protein that is substantially free of cellular material includes preparations of protein having less than about 30%, 20%, 10%, 5%, 1%, 0.5%, or 0.1% (total protein) of contaminating protein. When the protein of the invention or its biologically active portion is recombinantly produced, optimally culture medium represents less than about 30%, 20%, 10%, 5%, 1%, 0.5%, or 0.1% (by concentration) of chemical precursors or non-protein-of-interest chemicals.

The term “amino acid” refers to any monomeric unit that can be incorporated into a peptide, polypeptide, or protein. Amino acids include naturally-occurring α-amino acids and their stereoisomers, as well as unnatural (non-naturally occurring) amino acids and their stereoisomers. “Stereoisomers” of a given amino acid refer to isomers having the same molecular formula and intramolecular bonds but different three-dimensional arrangements of bonds and atoms (e.g., an L-amino acid and the corresponding D-amino acid).

Naturally-occurring amino acids are those encoded by the genetic code, as well as those amino acids that are later modified, e.g., hydroxyproline, γ-carboxyglutamate, and O-phosphoserine. Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and their combinations. Stereoisomers of a naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and their combinations.

Unnatural (non-naturally occurring) amino acids include, without limitation, amino acid analogs, amino acid mimetics, synthetic amino acids, N-substituted glycines, and N-methyl amino acids in either the L- or D-configuration that function in a manner similar to the naturally-occurring amino acids. For example, “amino acid analogs” can be unnatural amino acids that have the same basic chemical structure as naturally-occurring amino acids (i.e., a carbon that is bonded to a hydrogen, a carboxyl group, an amino group) but have modified side-chain groups or modified peptide backbones, e.g., homoserine, norleucine, methionine sulfoxide, methionine methyl sulfonium. “Amino acid mimetics” refer to chemical compounds that have a structure that is different from the general chemical structure of an amino acid, but that functions in a manner similar to a naturally-occurring amino acid. Amino acids may be referred to by either the commonly known three letter symbols or by the one-letter symbols recommended by the IUPAC-IUB Biochemical Nomenclature Commission.

The expression “conservatively modified variant” and related expression may apply to amino acid sequences, as well to nucleic acid sequences encoding amino acid sequence. Substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M).

The terms “nucleic acid,” “nucleic acid sequence,” “nucleotide sequence,” “oligonucleotide,” “polynucleotide” and the related terms and expressions refer to deoxyribonucleic acids (DNA) or ribonucleic acids (RNA) and their polymers. Nucleic acid sequences, as discussed in the present disclosure, encompass all forms of nucleic acids, including, but not limited to, single-stranded forms, double-stranded forms, hairpins, stem-and-loop structures, and the like. When an RNA sequence is described, its corresponding DNA sequence is also described, wherein uridine is represented as thymidine. When a DNA sequence is described, its corresponding RNA sequence is also described, wherein thymidine is represented as uridine. Unless specifically limited, the term “nucleic acid” and the related terms and expressions encompass nucleic acids containing known analogues of natural nucleotides that have similar properties as the reference nucleic acid, and are metabolized in a manner similar to naturally occurring nucleotides. A nucleic acid sequence can include combinations of deoxyribonucleic acids and ribonucleic acids. Such deoxyribonucleic acids and ribonucleic acids include both naturally occurring molecules and synthetic analogues. Unless otherwise indicated, a particular nucleic acid sequence also implicitly encompasses degenerate codon substitutions, alleles, orthologs, SNPs, and complementary sequences as well as the sequence explicitly indicated. Degenerate codon substitutions may be achieved by generating sequences in which the third position of one or more selected (or all) codons is substituted with mixed-base and/or deoxyinosine residues.

The terms “identity,” “substantial identity,” “similarity,” “substantial similarity,” “homology” and the related terms and expressions used in the context of describing nucleic acid or amino acid sequences refer to a sequence that has at least 60% sequence identity to a reference sequence. Examples include at least: 60%, 65%, 70%, 75%, 80%, 85%, 86%, 87%, 88%, 89%, 90%, 91%, 92%, 93%, 94%, 95%, 96%, 97%, 98%, or 99%, sequence identity, as compared to a reference sequence using the programs for comparison of nucleic acid or amino acid sequences, such as BLAST using standard parameters. For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default (standard) program parameters can be used, or alternative parameters can be designated. The sequence comparison algorithm then calculates the percent sequence identities for the test sequences relative to the reference sequence, based on the program parameters. A “comparison window” includes reference to a segment of any one of the number of contiguous positions (from 20 to 600, usually about 50 to about 200, more commonly about 100 to about 150), in which a sequence may be compared to a reference sequence of the same number of contiguous positions after the two sequences are optimally aligned. Methods of alignment of sequences for comparison are well-known. Optimal alignment of sequences for comparison may be conducted, for example, by the local homology algorithm of Smith and Waterman, 1981, by the homology alignment algorithm of Needleman and Wunsch, 1970, by the search for similarity method of Pearson and Lipman, 1988, by computerized implementations of these algorithms (for example, BLAST), or by manual alignment and visual inspection.

Algorithms that are suitable for determining percent sequence identity and sequence similarity include BLAST and BLAST 2.0 algorithms, which are described in Altschul et al., 1990, and Altschul et al., 1977, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (Henikoff and Henikoff, 1989). The BLAST algorithm also performs a statistical analysis of the similarity between two sequences (Karlin and Altschul, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²1.

The term “antibody” and the related terms refer to an immunoglobulin or its fragment that binds to a particular spatial and polar organization of another molecule. Immunoglobulins include various classes and isotypes, such as IgA, IgD, IgE, IgG1, IgG2a, IgG2b and IgG3, IgG4, IgM, etc. An antibody can be monoclonal or recombinant, and can be prepared by laboratory techniques, such as by preparing continuous hybrid cell lines and collecting the secreted protein, or by cloning and expressing nucleotide sequences or their mutagenized versions coding at least for the amino acid sequences required for binding. The term “antibody” encompasses natural, artificially modified, and artificially generated antibody forms, such as humanized, human, single-chain, chimeric, synthetic, recombinant, hybrid, mutated, grafted, and in vitro generated antibodies and their fragments. The term “antibody” also includes composite forms including but not limited to fusion proteins containing an immunoglobulin moiety. “Antibody” also refers to non-quaternary antibody structures (such as camelids and camelid derivatives). Antibody fragments may include Fab, Fv and F(ab′)₂, Fab′, scFv, Fd, dAb, Fc, and the like. Antibodies may also be single-chain antibodies, chimeric antibodies, humanized antibodies, or any other antibody derivative that retains binding activity that is specific for a particular binding site. In addition, aggregates, polymers and conjugates of immunoglobulins or their fragments can be used where appropriate.

The expression “neutralizing antibody” can refer to an antibody capable of keeping an infectious agent, such as a virus, from infecting a cell by neutralizing or inhibiting one or more parts of the life cycle of the infectious agent. In the context of the present disclosure, neutralizing antibodies can prevent a coronavirus, such as, but not limited to, SARS-CoV-2, from completing its life cycle in host cell. The life cycle of the virus, for example, a coronavirus, starts with attachment of the virus to a host cell and ending with budding of newly formed virus from the host cell. This life cycle includes, but is not limited to, the steps of attaching to a cell, entering a cell, fusion of the viral membrane with the host cell membrane, release of viral ribonucleoproteins into the cytoplasm, formation of new viral particles and budding of viral particles from the host cell membrane

The term “immunogenic” and the related terms, when used in the context of the present disclosure, refers to the ability of an antigen, which can be a protein, a polypeptide, or a region of a protein or a polypeptide, to elicit in a subject an immune response to the specific antigen. In the context of the present disclosure, an immune response is the development in a subject of a humoral and/or a cellular immune response to an antigen. A “humoral immune response” refers to an immune response mediated by antibody molecules, including secretory (IgA) or IgG molecules, while a “cellular immune response” is one mediated by T-lymphocytes and/or other white blood cells. One important aspect of cellular immunity involves an antigen-specific response by cytolytic T-cells (“CTL”s). CTLs have specificity for peptide antigens that are presented in association with proteins encoded by the major histocompatibility complex (MHC) and expressed on the surfaces of cells. CTLs help induce and promote the destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A cellular immune response also refers to the production of cytokines, chemokines and other such molecules produced by activated T-cells and/or other white blood cells, including those derived from CD4⁺ and CD8⁺ T-cells. Thus, an immunogenic composition can stimulate CTLs, and/or the production or activation of helper T-cells. The production of chemokines and/or cytokines may also be stimulated. An immunogenic composition may also elicit an antibody-mediated immune response. An immunogenic composition may include one or more of the following effects upon administration to a subject: production of antibodies by B-cells; and/or the activation of suppressor, cytotoxic, or helper T-cells and/or T-cells directed specifically to a an antigen protein present in the immunogenic composition. Immune response elicited in the subject may serve to neutralize infectivity of a virus, such as a coronavirus, for example, SARS-CoV-2, and/or mediate antibody-complement, or antibody dependent cell cytotoxicity (ADCC) to provide protection against viral infection to an immunized subject. Various aspects of an immune response elicited by an immunogenic compositions can be determined using standard assays, some of which are described in the present disclosure.

Immunogenic compositions, as described in the present disclosure, may also be referred to as “vaccines.” Immunogenic compositions, or vaccines, may contain antigens that elicit immune response to them in a subject upon administration. For example, some immunogenic compositions, or vaccines, described in the present disclosure contain coronavirus Spike protein antigens, such as SARS-CoV-2 Spike protein antigens, that can elicit immune response to them in a subject upon administration. Immunogenic compositions may also contain nucleic acid sequences encoding such antigens. For example, some immunogenic compositions, or vaccines, described in the present disclosure contain nucleic acid sequences encoding coronavirus Spike protein antigens, such as SARS-CoV-2 Spike protein antigens. Immunogenic compositions containing antigen-encoding nucleic acid sequences may be described or referred to as “nucleic acid vaccines.” An expression “nucleic acid vaccine” and the related term and expressions encompasses naked DNA vaccines, e.g., plasmid vaccine, and viral vector-based nucleic acids vaccines that are comprised by a viral vector and/or delivered as viral particles.

The term “antigen” refers to a molecule, such as a polypeptide, containing one or more epitopes (either linear, conformational or both) that can stimulate a subject's immune system to produce antigen-specific immune response. A polypeptide epitope may include between about 7 and 15 amino acids, such as, 9, 10, 12 or 15 amino acids. For example, the expression “coronavirus Spike protein antigen” may refer to a polypeptide of a coronavirus Spike protein, such as SARS-CoV-2 Spike protein. The term “antigen” may be used interchangeably with the term “immunogen.”

“Virus” is used in both the plural and singular senses. “Virion” refers to a single virus. For example, the expression “coronavirus virion” refers to a coronavirus particle.

Coronaviruses are a group of enveloped, single-stranded RNA viruses that cause diseases in mammals and birds. Coronavirus hosts include bats, pigs, dogs, cats, mice, rats, cows, rabbits, chickens and turkeys. In humans, coronaviruses cause mild to severe respiratory tract infections. Coronaviruses vary significantly in risk factor. Some can kill more than 30% of infected subjects. Some examples of human coronaviruses are: Human coronavirus 229E (HCoV-229E); Human coronavirus OC43 (HCoV-OC43); Severe acute respiratory syndrome coronavirus (SARS-CoV); Human coronavirus NL63 (HCoV-NL63, New Haven coronavirus); Human coronavirus HKU1 (HCoV-HKU1), which originated from infected mice, was first discovered in January 2005 in two patients in Hong Kong; Middle East respiratory syndrome-related coronavirus (MERS-CoV), also known as novel coronavirus 2012 and HCoV-EMC; and Severe acute respiratory syndrome coronavirus 2 (SARS-CoV-2), also known as 2019-nCoV or “novel coronavirus 2019” (Wu et al., 2020). In human, SARS-CoV-2 causes coronavirus disease termed COVID-19, which can cause severe symptoms and death.

Spike protein (or “S protein”) is a coronavirus surface proteins that is able to mediate receptor binding and membrane fusion between a coronavirus virion and its host cell. Characteristic spikes on the surface of coronavirus virions are formed by ectodomains of homotrimers of Spike protein. Coronavirus Spike protein is highly glycosylated, with different versions containing 21 to 35 N-glycosylation sites. In comparison to trimeric glycoproteins found on other human-pathogenic enveloped RNA viruses, coronavirus Spike protein is considerably larger, and totals nearly 700 kDa per trimer. Ectodomains of coronavirus Spike proteins contain an a N-terminal domain named S1, which is responsible for binding of receptors on the host cell surface, and a C-terminal S2 domain responsible for fusion. S1 domain of SARS-CoV-2 Spike protein is able to bind to Angiotensin-converting enzyme 2 (ACE2) of host cells. The region of SARS-CoV-2 Spike protein S1 domain that recognizes ACE2 is a 25 kDa domain called the receptor binding domain (RBD) (Walls et al., 2020). When expressed as a stand-alone polypeptide, the RBD can form a functionally folded domain capable of binding ACE2. In different coronaviruses, Spike proteins may or may not be cleaved during assembly and exocytosis of virions. In most alphacoronaviruses, and in betacoronavirus SARS-CoV, the virions harbor uncleaved Spike protein, whereas in virions of some betacoronaviruses, including SARS-CoV-2, and in known gammacoronaviruses, Spike protein is found cleaved between the S1 and S2 domains. In these virions, Spike protein is typically cleaved by furin, a Golgi-resident host protease. Accordingly, naturally occurring or “wild-type” amino acid sequence of Spike protein of SARS-CoV-2 (which is considered to be the sequence of the first virus SARS-CoV-2 isolate, Wuhan-Hu-1), contains a furin cleavage site between S1 and S2 domains. S2 domain of coronavirus Spike proteins contain two heptad repeats, HR1 and HR2, which contain a repetitive heptapeptide characteristic of the formation of coiled-coil that participate in the fusion process. Analysis of sera from COVID-19 patients demonstrates that antibodies are elicited against the Spike protein and can inhibit viral entry into the host cell (Brouwer et al., 2020). The first Cryo-EM structure of SARS-CoV-2 Spike protein is described in Wrapp et al., 2020.

“Wild-type” amino acid sequence of
Spike protein of SARS-CoV-2-
SEQ ID NO: 1
MFVFLVLLPLVSSQCVNLTTRTQLPPAYTNSFTRGVYYPDKVFRSSVLHSTQDLFLPFFSNV
TWFHAIHVSGTNGTKRFDNPVLPENDGVYFASTEKSNIIRGWIFGTTLDSKTQSLLIVNNAT
NVVIKVCEFQFCNDPFLGVYYHKNNKSWMESEFRVYSSANNCTFEYVSQPFLMDLEGKQGNF
KNLREFVFKNIDGYFKIYSKHTPINLVRDLPQGFSALEPLVDLPIGINITRFQTLLALHRSY
LTPGDSSSGWTAGAAAYYVGYLQPRTELLKYNENGTITDAVDCALDPLSETKCTLKSFTVEK
GIYQTSNERVQPTESIVRFPNITNLCPFGEVENATRFASVYAWNRKRISNCVADYSVLYNSA
SFSTFKCYGVSPTKLNDLCFTNVYADSFVIRGDEVRQIAPGQTGKIADYNYKLPDDFTGCVI
AWNSNNLDSKVGGNYNYLYRLERKSNLKPFERDISTEIYQAGSTPCNGVEGENCYFPLQSYG
FQPTNGVGYQPYRVVVLSFELLHAPATVCGPKKSTNLVKNKCVNFNENGLTGTGVLTESNKK
FLPFQQFGRDIADTTDAVRDPQTLEILDITPCSFGGVSVITPGTNTSNQVAVLYQDVNCTEV
PVAIHADQLTPTWRVYSTGSNVFQTRAGCLIGAEHVNNSYECDIPIGAGICASYQTQTNSPR
RARSVASQSIIAYTMSLGAENSVAYSNNSIAIPTNFTISVTTEILPVSMTKTSVDCTMYICG
DSTECSNLLLQYGSFCTQLNRALTGIAVEQDKNTQEVFAQVKQIYKTPPIKDFGGFNFSQIL
PDPSKPSKRSFIEDLLENKVTLADAGFIKQYGDCLGDIAARDLICAQKENGLTVLPPLLTDE
MIAQYTSALLAGTITSGWTFGAGAALQIPFAMQMAYRENGIGVTQNVLYENQKLIANQFNSA
IGKIQDSLSSTASALGKLQDVVNQNAQALNTLVKQLSSNFGAISSVLNDILSRLDKVEAEVQ
IDRLITGRLQSLQTYVTQQLIRAAEIRASANLAATKMSECVLGQSKRVDFCGKGYHLMSFPQ
SAPHGVVFLHVTYVPAQEKNFTTAPAICHDGKAHFPREGVFVSNGTHWFVTQRNFYEPQIIT
TDNTFVSGNCDVVIGIVNNTVYDPLQPELDSFKEELDKYFKNHTSPDVDLGDISGINASVVN
IQKEIDRLNEVAKNLNESLIDLQELGKYEQYIKWPWYIWLGFIAGLIAIVMVTIMLCCMTSC
CSCLKGCCSCGSCCKFDEDDSEPVLKGVKLHYT

A “domain” of a protein or a polypeptide refers to a region of the protein or polypeptide defined by structural and/or a functional properties. Exemplary function properties include enzymatic activity and/or the ability to bind to or be bound by another protein or non-protein entity. For example, coronavirus Spike protein contains S1 and S2 domains.

The term “oligomer” and related terms, when used in reference to polypeptides or proteins, refer to complexes formed by two or more polypeptide or protein monomers, which can also be referred to as “subunits” or “chains.” For example, a trimer is an oligomer formed by three polypeptide subunits.

The terms “fusion protein,” “fusion polypeptide,” and the related terms relate to polypeptide molecules, including artificial or engineered polypeptide molecules, that include two or more amino acid sequences previously found in separate polypeptide molecule, that are joined or linked in a fusion protein amino acid sequence to form a single polypeptide. For example, a fusion protein can be an engineered recombinant protein containing amino acid sequence from at least two unrelated proteins that have been joined together, via a peptide bond, to make a single protein. In this context, proteins are considered unrelated, if their amino acid sequences are not normally found joined together via a peptide bond in their natural environment, for example, inside a cell. For example, the present disclosure describes fusion proteins that include an amino acid sequence of a Spike protein of a coronavirus and an amino acid sequence of a ferritin subunit polypeptide, which are unrelated proteins. The amino acid sequences of a fusion protein are encoded by corresponding nucleic acid sequences that are joined “in frame,” so that they are transcribed and translated to produce a single polypeptide. The amino acid sequences of a fusion protein can be contiguous or separated by one or more spacer, linker or hinge sequences. Fusion proteins can include additional amino acid sequences, such as, for example, signal sequences, tag sequences, and/or linker sequences.

Ferritin is a globular protein found in animals, bacteria, and plants, that acts primarily to control the rate and location of polynuclear Fe(III)₂O₃ formation through the transportation of hydrated iron ions and protons to and from a mineralized core. The globular form of ferritin is made up of monomeric subunit proteins (also referred to as monomeric ferritin subunits), which are polypeptides having a molecule weight of approximately 17-20 kDa. An example of the sequence of one such monomeric ferritin subunit is represented by SEQ ID NO:2. Each monomeric ferritin subunit has the topology of a helix bundle which includes a four antiparallel helix motif, with a fifth shorter helix (the c-terminal helix) lying roughly perpendicular to the long axis of the 4 helix bundle. According to convention, the helices are labeled ‘A, B, C, and D & E’ from the N-terminus respectively. The N-terminal sequence lies adjacent to the capsid three-fold axis and extends to the surface, while the E helices pack together at the four-fold axis with the C-terminus extending into the particle core. The consequence of this packing creates two pores on the capsid surface. It is expected that one or both of these pores represent the point by which the hydrated iron diffuses into and out of the capsid. Following production, these monomeric ferritin subunit proteins self-assemble into the globular ferritin protein. Thus, the globular form of ferritin comprises 24 monomeric, ferritin subunit proteins, and has a capsid-like structure having 432 symmetry.

Amino acid sequence of Helicobacter pylori
ferritin subunit with the N-terminal deletion
of the first five amino acids-
SEQ ID NO: 2
DIIKLLNEQVNKEMQSSNLYMSMSSWCYTHSLDGAGLFLFD
HAAEEYEHAKKLIIFLNENNVPVQLTSISAPEHKFEGLTQI
FQKAYEHEQHISESINNIVDHAIKSKDHATFNFLQWYVAEQ
HEEEVLFKDILDKIELIGNENHGLYLADQYVKGIAKSRKS

The terms “individual”, “subject”, and “patient” can be used interchangeably in the present disclosure to refer to a non-human animal or a human. Examples of subjects include, but are not limited to: humans and other primates, including non-human primates, such as chimpanzees and other apes and monkey species; farm animals, such as cattle, sheep, pigs, seals, goats and horses; domestic mammals such as dogs and cats; laboratory animals including rodents, such as mice, rats and guinea pigs; birds, including domestic, wild and game birds, such as chickens, turkeys and other gallinaceous birds, ducks, geese, and the like. The terms individual, subject, and patient, by themselves, do not denote a particular age, sex, race, or clinical status. Thus, subjects of any age, whether male or female, are intended to be covered by the present disclosure and include, but are not limited to the elderly, adults, children, babies, infants, and toddlers. Likewise, the methods of the present invention can be applied to any human race, including, for example, Caucasian (white), African-American (black), Native American, Native Hawaiian, Hispanic, Latino, Asian, and European. An infected subject is a subject that is known to have been infected by an infections organism, such as coronavirus, for example SARS-CoV-2.

The terms “administering” or “administration,” when using in the context of administration of a composition described in the present disclosure to a subject (and the related terms and expression), refer to the act of physically delivering a substance as it exists outside the body (for example, an immunogenic composition described in the present disclosure) into a subject. Administration can be by mucosal, intradermal, intravenous, intramuscular, subcutaneous delivery and/or by any other known methods of physical delivery. Administration encompasses direct administration, such as administration to a subject by a medical professional or self-administration, or indirect administration, which may be the act of prescribing a composition described in the present disclosure.

The term “glycosylation” and the related terms and expression refer to a process and/or result of post-translational modification of proteins and polypeptides that adds carbohydrate moieties (also referred to as “glycans”) to certain amino acids of a polypeptide or protein molecules. In N-linked glycosylation, a carbohydrate moiety is added to asparagine. In O-linked glycosylation, a carbohydrate moiety is added to serine or threonine. Attachment of the carbohydrate moiety requires recognition of a consensus amino acid sequence (“consensus sequence”).

### Fusion Proteins and Nanoparticles

Provided in this disclosure and included among the embodiments of the present invention are fusion proteins comprising an amino acid sequence of a Spike protein of a coronavirus (“coronavirus Spike protein”) and an amino acid sequence of a ferritin subunit polypeptide. Coronavirus Spike protein amino acid sequence included in the fusion proteins according to the embodiments of the present invention may also be referred to as “Spike polypeptide,” “Spike protein domain” or “Spike domain,” while the ferritin subunit polypeptide amino acid sequence may be referred to as “ferritin amino acid sequence,” “ferritin”, “ferritin domain”, or “ferritin polypeptide.” In addition to the above amino acid sequences, fusion proteins according to the embodiments of the present invention can include other amino acid sequences such as, but not limited to, amino acid sequence of polypeptide domains other than Spike domain and ferritin domains, linker sequences, signal sequences, tags, etc. Some of these other amino acid sequences are described elsewhere in the present disclosure.

An amino acid sequence of a coronavirus Spike protein included in a fusion protein according to embodiments of the present invention can be a Spike protein sequence from any coronavirus, such as an alphacoronavirus, a betacoronoviurs, a gammacoronovirus, or a deltacoronavirus. Some embodiments of the fusion proteins described in the present disclosure include an amino acid sequence of a Spike protein of a coronavirus capable of infecting humans (“human coronaviruses”), including, but not limited to, human betacoronaviruses, for example, SARS-CoV, MERS-CoV, and SARS-CoV-2. Some embodiments of the fusion proteins described in the present disclosure include an amino acid sequence of a Spike protein of a coronavirus capable of infecting non-human animals including, but not limited to, BatCoV RaTG13, Bat SARSr-CoV ZXC21, Bat SARSr-CoV ZC45, BatSARSr-CoV WIV1, or other coronaviruses described, for example, in Zhang et al., 2020. It is to be understood that a coronavirus Spike protein sequence may be a full or a partial amino acid sequence of a Spike protein, an amino acid sequence of a fragment of a Spike protein, or an amino acid sequence of a variant of a Spike protein, including naturally occurring and artificially generated variants. Some of exemplary variants of Spike protein amino acid sequences are variants found in naturally circulating SARS-CoV-2 variants, such as, but not limited to, variants D614G, B.1.1.7 (also known as “alpha variant”), B.1.429 (also known as “LA variant”), P1 (also known as “gamma variant”), and B.1.351 (also known as “beta variant”), or B.1.617.2 (also known as “delta variant”).

Some embodiments of the fusion proteins may contain a naturally occurring (or “wild-type”) amino acid sequence of coronavirus Spike proteins or a portion thereof. Some non-limiting examples of such wild-type sequences are: a wild-type amino acid sequence of S1 domain of a coronavirus Spike protein; a wild-type amino acid sequence of an RBD domain of a coronavirus Spike protein; or a wild-type amino acid sequence of a coronavirus Spike protein with one or more C-terminal, N-terminal, or middle portions deleted. One example is a wild-type amino acid sequence of a coronavirus Spike protein with a C-terminal deletion encompassing the HR2 amino acid sequence. Some other examples of wild-type amino acid sequences of a coronavirus Spike protein are the sequences that contain mutations, in comparison to SEQ ID NO:1, found in naturally occurring SARS-CoV-2 strains, which can also be referred to as “variants.” One such example is a wild-type amino acid sequence of a coronavirus Spike protein having a deletion (in reference to SEQ ID NO:1) of residues 69-70 and residue 144, as found in strain SARS-CoV-2 VUI 202012/01 in SARS-CoV-2 variant lineage B.1.1.7. One more example is a wild-type amino acid sequence of a coronavirus Spike protein having a D to G substitution at residue 614, (in reference to SEQ ID NO:1), as found in SARS-CoV-2 variant D614G. One more example is a wild-type amino acid sequence of a coronavirus Spike protein having the substitutions (in reference to SEQ ID NO:1) S13I, W152C, L452R, and D614G, as found in SARS-CoV-2 variant B.1.429. Another example is a wild-type amino acid sequence of a coronavirus Spike protein having substitutions (in reference to SEQ ID NO:1) L18F, T20N, P26S, D138Y, R190S, K417T, E484K, N501Y, D614G, H655Y, T1027I, as found in SARS-CoV-2 variant P1. Yet another example is a wild-type amino acid sequence of a coronavirus Spike protein having substitutions (in reference to SEQ ID NO:1) L18F, D80A, D215G, 242-244 del, R246I, K417N, E484K, N501Y, D614G, A701V, as found in SARS-CoV-2 variant B.1.351. One more example is a wild-type amino acid sequence of a coronavirus Spike protein having a deletion (in reference to SEQ ID NO:1) of residues 69-70 and residue 144, and substitutions (in reference to SEQ ID NO:1) N501Y, A570D, D614G, P681H, T716I, S982A, D1118H, as found in SARS-CoV-2 variant B.1.1.7. One more example is a wild-type amino acid sequence of a coronavirus Spike protein having a deletion (in reference to SEQ ID NO:1) of residues 156-157, and substitutions (in reference to SEQ ID NO:1) T19R, G142D, R158G, L452R, T478K, D614G, P681R, and D950N, as found in SARS-CoV-2 variant B.1.617.2. An additional examples include the sequence of other naturally occurring strains having a deletion of a few residues (e.g., 1-5) within the coronavirus Spike protein before HR2 amino acid sequence. Some of the features of the above amino acid sequences of a coronavirus Spike protein are summarized in Table 1. It is to be understood that, in some examples of SARS-CoV-2 Spike protein antigens according to the present disclosure, various features and mutations of the wild-type amino acid sequences of a coronavirus Spike protein, including but not limited to those discussed above and summarized above, can be found in various combinations and subcombinations.

TABLE 1
Exemplary features (in reference to SEQ ID NO: 1) found
in amino acid sequences of a coronavirus Spike protein.

| SARS- | SARS- | SARS- | SARS- | SARS- | SARS- |
|---|---|---|---|---|---|
| CoV-2 | CoV-2 | CoV-2 | CoV-2 | CoV-2 | CoV-2 |
| D614G | B.1.1.7 | B.1.351 | P.1 | B.1.429 | B.1.617.2 |
| D614G | 69-70 del | L18F | L18F | S13I | T19R |
| | 144 del | D80A | T20N | W152C | G142D |
| | N501Y | D215G | P26S | L452R | 156-157 del |
| | A570D | 242-244 del | D138Y | D614G | R158G |
| | D614G | R246I | R190S | | L452R |
| | P681H | K417N | K417T | | T478K |
| | T716I | E484K | E484K | | D614G |
| | S982A | N501Y | N501Y | | P681R |
| | D1118H | D614G | D614G | | D950N |
| | | A701V | H655Y | | |
| | | | T1027I | | |

Some embodiments of the fusion proteins may contain artificially modified amino acid sequences of coronavirus Spike proteins or portion thereof. In some non-limiting examples, artificially modified amino acid sequences may contain one or more features of the wild-type amino acid sequences of a coronavirus Spike protein sequences, such as, but not limited to, those discussed in the present disclosure. In some exemplary embodiments, the features of the wild-type amino acid sequences of a coronavirus Spike protein sequences may be combined in ways that are not found naturally occurring sequence. For example, an artificially modified amino acid sequence of coronavirus Spike proteins or portion thereof or a portion thereof may include one or more features from each of two or more naturally circulating SARS-CoV-2 variants, such as, but not limited to, variants D614G, B.1.1.7, B.1.429, B.1.351, P1, and B.1.617.2, Some other non-limiting examples of such artificially modified sequences are: an artificially modified amino acid sequence of S1 domain of a coronavirus Spike protein; an artificially modified amino acid sequence of an RBD domain of a coronavirus Spike protein; or an artificially modified amino acid sequence of a coronavirus Spike protein with one or more C-terminal, N-terminal, or middle portions deleted, such as an artificially modified amino acid sequence of a coronavirus Spike protein with a C-terminal deletion encompassing the HR2 amino acid sequence. Some exemplary embodiments of fusion proteins contain coronavirus Spike protein amino acid sequences, naturally occurring or artificially modified, with a C-terminal deletion in S2 domain encompassing HR2 amino acid sequence. For example, a coronavirus Spike protein amino acid sequence may contain a deletion of HR2 amino acid sequence or a deletion of 70 or fewer, 60 or fewer, or 50 or fewer, for example, 50 to 70, of C-terminal amino acids of the S2 domain. Artificially modified amino acid sequences of coronavirus Spike proteins may contain various amino acid modifications, as compared wild-type sequences. For example, an artificially modified amino acid sequence of a coronavirus Spike protein may contain mutations removing or adding glycosylation sites. In another example, an artificially modified amino acid sequence of a coronavirus Spike protein may contain one or more mutations eliminating a protease recognition site, such as furin recognition site. In another example, an artificially modified amino acid sequence of a coronavirus Spike protein may contain one or more mutations affecting a conformation of a Spike domain, such as mutations stabilizing a Spike domain in a pre-fusion conformation. Some exemplary modifications of wild-type SARS-CoV-2 Spike protein sequence are described, for example, in Amanat et al., 2020 and Hhsieh et al., 2020. SEQ ID NO:3, described in Amanat et al., 2020, is an artificially modified SARS-CoV-2 Spike protein sequence with a furin cleavage site PRAR sequence mutated to alanine (residue 667 in SEQ ID NOs 1 and 3) and proline substitutions at residues 968 and 969 of SEQ ID NO:1. SEQ ID NO:14, described in Hhsieh et al., 2020, is an artificially modified SARS-CoV-2 Spike protein sequence (“HexaPro”) with six proline substitutions: F817P, A892P, A899P, A942P (all denoted with respect to SEQ ID NO:1), and proline substitutions at residues 968 and 969 of SEQ ID NO:1.

Artificially modified SARS-CoV-2 Spike protein
sequence-SEQ ID NO: 3;
mutation of PRAR furin cleavage site to
alanine and proline substitutions are
shown in bold
CVNLTTRTQLPPAYTNSFTRGVYYPDKVFRSSVLHSTQDLFLPFFSNVTWFHAIHVSGINGT
KRFDNPVLPENDGVYFASTEKSNIIRGWIFGTTLDSKTQSLLIVNNATNVVIKVCEFQFCND
PFLGVYYHKNNKSWMESEFRVYSSANNCTFEYVSQPFLMDLEGKQGNEKNLREFVEKNIDGY
FKIYSKHTPINLVRDLPQGFSALEPLVDLPIGINITRFQTLLALHRSYLTPGDSSSGWTAGA
AAYYVGYLQPRTFLLKYNENGTITDAVDCALDPLSETKCTLKSFTVEKGIYQTSNERVQPTE
SIVRFPNITNLCPFGEVENATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVGGN
YNYLYRLFRKSNLKPFERDISTEIYQAGSTPCNGVEGENCYFPLQSYGFQPTNGVGYQPYRV
VVLSFELLHAPATVCGPKKSTNLVKNKCVNFNFNGLTGTGVLTESNKKELPFQQFGRDIADT
TDAVRDPQTLEILDITPCSFGGVSVITPGTNTSNQVAVLYQDVNCTEVPVAIHADQLTPTWR
VYSTGSNVFQTRAGCLIGAEHVNNSYECDIPIGAGICASYQTQTNSPASVASQSIIAYTMSL
GAENSVAYSNNSIAIPTNFTISVTTEILPVSMTKTSVDCTMYICGDSTECSNLLLQYGSFCT
QLNRALTGIAVEQDKNTQEVFAQVKQIYKTPPIKDFGGENFSQILPDPSKPSKRSFIEDLLF
NKVTLADAGFIKQYGDCLGDIAARDLICAQKENGLTVLPPLLTDEMIAQYTSALLAGTITSG
WTFGAGAALQIPFAMQMAYRENGIGVTQNVLYENQKLIANQFNSAIGKIQDSLSSTASALGK
LQDVVNQNAQALNTLVKQLSSNFGAISSVLNDILSRLDPPEAEVQIDRLITGRLQSLQTYVT
QQLIRAAEIRASANLAATKMSECVLGQSKRVDFCGKGYHLMSFPQSAPHGVVFLHVTYVPAQ
EKNFTTAPAICHDGKAHFPREGVFVSNGTHWFVTQRNFYEPQIITTDNTFVSGNCDVVIGIV
NNTVYDPLQPELDSFKEELDKYFKNHTSPDVDLGDISGINASVVNIQKEIDRLNEVAKNLNE
SLIDLQELGKYEQYIKWPSGR
Artificially modified SARS-CoV-2 Spike protein
sequence “HexaPro”-SEQ ID NO: 14;
proline substitutions are shown in bold
CVNLTTRTQLPPAYTNSFTRGVYYPDKVFRSSVLHSTQDLFLPFFSNVTWFHAIHVSGTNGT
KRFDNPVLPFNDGVYFASTEKSNIIRGWIFGTTLDSKTQSLLIVNNATNVVIKVCEFQFCND
PFLGVYYHKNNKSWMESEFRVYSSANNCTFEYVSQPFLMDLEGKQGNFKNLREFVEKNIDGY
FKIYSKHTPINLVRDLPQGFSALEPLVDLPIGINITRFQTLLALHRSYLTPGDSSSGWTAGA
AAYYVGYLQPRTFLLKYNENGTITDAVDCALDPLSETKCTLKSFTVEKGIYQTSNERVQPTE
SIVRFPNITNLCPFGEVENATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVGGN
YNYLYRLFRKSNLKPFERDISTEIYQAGSTPCNGVEGENCYFPLQSYGFQPTNGVGYQPYRV
VVLSFELLHAPATVCGPKKSTNLVKNKCVNFNFNGLTGTGVLTESNKKELPFQQFGRDIADT
TDAVRDPQTLEILDITPCSFGGVSVITPGTNTSNQVAVLYQDVNCTEVPVAIHADQLTPTWR
VYSTGSNVFQTRAGCLIGAEHVNNSYECDIPIGAGICASYQTQTNSPGSASSVASQSIIAYT
MSLGAENSVAYSNNSIAIPTNFTISVTTEILPVSMTKTSVDCTMYICGDSTECSNLLLQYGS
FCTQLNRALTGIAVEQDKNTQEVFAQVKQIYKTPPIKDFGGFNFSQILPDPSKPSKRSPIED
LLFNKVTLADAGFIKQYGDCLGDIAARDLICAQKENGLTVLPPLLTDEMIAQYTSALLAGTI
TSGWTFGAGPALQIPFPMQMAYRENGIGVTQNVLYENQKLIANQFNSAIGKIQDSLSSTPSA
LGKLQDVVNQNAQALNTLVKQLSSNFGAISSVLNDILSRLDPPEAEVQIDRLITGRLQSLQT
YVTQQLIRAAEIRASANLAATKMSECVLGQSKRVDFCGKGYHLMSFPQSAPHGVVFLHVTYV
PAQEKNFTTAPAICHDGKAHFPREGVFVSNGTHWFVTQRNFYEPQIITTDNTFVSGNCDVVI
GIVNNTVYDPLQPELDSFKEELDKYFKNHTSPDVDLGDISGINASVVNIQKEIDRLNEVAKN
LNESLIDLQELGKYEQYIKWPSGR

In some embodiments, the amino acid sequence of a Spike protein of a coronavirus included in a fusion protein as provided herein is an amino acid sequence with at least 80%, at least 85%, at least 90%, at least 95%, at least 96%, at least 97%, at least 98%, or at least 99% sequence identity to a wild-type or artificially modified amino acid sequence of SARS-CoV-2 Spike protein amino acid sequence. In some embodiments, the amino acid sequence of a Spike protein of a coronavirus included in a fusion protein as provided herein is an amino acid sequence with at least 80%, at least 85%, at least 90%, at least 95%, at least 96%, at least 97%, at least 98%, or at least 99% sequence identity to a portion of the amino acid sequence of wild-type or artificially modified SARS-CoV-2 Spike protein amino acid sequence. In some instances, the Spike protein of a coronavirus included in a fusion protein as provided herein is a conservatively modified variant Spike protein comprising one or more amino acid residue substitutions. In some instances, the Spike protein of a coronavirus included in a fusion protein as provided herein comprises a deletion of one or more amino acid residues at the C-terminal, N-terminal, and/or middle portion of the protein. In some instances, the deletion may comprise a one or more consecutive amino acid residues. In some instances, the deletion may comprise a one or more non-consecutive amino acid residues. In some instances, the Spike protein may comprise a deletion of 1, 2, 3, 4, 5, 6, 7, 8, 9, or 10 amino acid residues. In some instances, the Spike protein may comprise a deletion of 10, 15, 20, 25, 30, 35, 40, 45, 50, 55, 60, 65, 70, 75, 80, 85, 90, 95, or 100 amino acid residues, such as deletions of 10-15, 15-30, 25-50, 10-50, or 50-100 amino acid residues. For example, the amino acid sequence of a Spike protein of a coronavirus included in a fusion protein as provided herein may be a sequence with at least 80%, at least 85%, at least 90%, at least 95%, at least 96%, at least 97%, at least 98%, or at least 99% sequence identity to residues 15 to 1146 of SEQ ID NO:1, residues 15 to 1213 of SEQ ID NO:1, or residues 1 to 1146 of SEQ ID NO:1. In some embodiments, an amino acid sequence of a Spike protein of a coronavirus included in a fusion protein as provided herein is a sequence with at least 80%, at least 85%, at least 90%, at least 95%, at least 96%, at least 97%, at least 98%, or at least 99% sequence identity to SEQ ID NO:3. In some embodiments, an amino acid sequence of a Spike protein of a coronavirus included in a fusion protein as provided herein is a sequence with at least 80%, at least 85%, at least 90%, at least 95%, at least 96%, at least 97%, at least 98%, or at least 99% sequence identity to SEQ ID NO:4. In some embodiments, an amino acid sequence of a Spike protein of a coronavirus included in a fusion protein as provided herein is a sequence with at least 80%, at least 85%, at least 90%, at least 95%, at least 96%, at least 97%, at least 98%, or at least 99% sequence identity to SEQ ID NO:14. In some embodiments, an amino acid sequence of a Spike protein of a coronavirus included in a fusion protein as provided herein is a sequence with at least 80%, at least 85%, at least 90%, at least 95%, at least 96%, at least 97%, at least 98%, or at least 99% sequence identity to SEQ ID NO:15.

Artificially modified partial SARS-CoV-2 Spike protein sequence-
SEQ ID NO: 4
CVNLTTRTQLPPAYTNSFTRGVYYPDKVFRSSVLHSTQDLFLPFFSNVTWFHAIHVSGTNGT
KRFDNPVLPENDGVYFASTEKSNIIRGWIFGTTLDSKTQSLLIVNNATNVVIKVCEFQFCND
PFLGVYYHKNNKSWMESEFRVYSSANNCTFEYVSQPFLMDLEGKQGNFKNLREFVEKNIDGY
FKIYSKHTPINLVRDLPQGFSALEPLVDLPIGINITRFQTLLALHRSYLTPGDSSSGWTAGA
AAYYVGYLQPRTFLLKYNENGTITDAVDCALDPLSETKCTLKSFTVEKGIYQTSNERVQPTE
SIVRFPNITNLCPFGEVENATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVGGN
YNYLYRLERKSNLKPFERDISTEIYQAGSTPCNGVEGENCYFPLQSYGFQPINGVGYQPYRV
VVLSFELLHAPATVCGPKKSTNLVKNKCVNFNFNGLTGTGVLTESNKKELPFQQFGRDIADT
TDAVRDPQTLEILDITPCSFGGVSVITPGTNTSNQVAVLYQDVNCTEVPVAIHADQLTPTWR
VYSTGSNVFQTRAGCLIGAEHVNNSYECDIPIGAGICASYQTQTNSPASVASQSIIAYTMSL
GAENSVAYSNNSIAIPTNFTISVTTEILPVSMTKTSVDCTMYICGDSTECSNLLLQYGSFCT
QLNRALTGIAVEQDKNTQEVFAQVKQIYKTPPIKDFGGENFSQILPDPSKPSKRSFIEDLLF
NKVTLADAGFIKQYGDCLGDIAARDLICAQKENGLTVLPPLLTDEMIAQYTSALLAGTITSG
WTFGAGAALQIPFAMQMAYRENGIGVTQNVLYENQKLIANQFNSAIGKIQDSLSSTASALGK
LQDVVNQNAQALNTLVKQLSSNFGAISSVLNDILSRLDPPEAEVQIDRLITGRLQSLQTYVT
QQLIRAAEIRASANLAATKMSECVLGQSKRVDFCGKGYHLMSFPQSAPHGVVFLHVTYVPAQ
EKNFTTAPAICHDGKAHFPREGVFVSNGTHWFVTQRNFYEPQIITTDNTFVSGNCDVVIGIV
NNTVYDPLQPELD
Artificially modified partial SARS-CoV-2 Spike protein sequence-
SEQ ID NO: 15
CVNLTTRTQLPPAYTNSFTRGVYYPDKVFRSSVLHSTQDLFLPFFSNVTWFHAIHVSGTNGT
KRFDNPVLPFNDGVYFASTEKSNIIRGWIFGTTLDSKTQSLLIVNNATNVVIKVCEFQFCND
PFLGVYYHKNNKSWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLTPGDSSSGWTAGA
AAYYVGYLQPRTFLLKYNENGTITDAVDCALDPLSETKCTLKSFTVEKGIYQTSNERVQPTE
SIVRFPNITNLCPFGEVENATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVGGN
YNYLYRLFRKSNLKPFERDISTEIYQAGSTPCNGVEGENCYFPLQSYGFQPTNGVGYQPYRV
VVLSFELLHAPATVCGPKKSTNLVKNKCVNFNFNGLTGTGVLTESNKKELPFQQFGRDIADT
TDAVRDPQTLEILDITPCSFGGVSVITPGTNTSNQVAVLYQDVNCTEVPVAIHADQLTPTWR
VYSTGSNVFQTRAGCLIGAEHVNNSYECDIPIGAGICASYQTQTNSPGSASSVASQSIIAYT
MSLGAENSVAYSNNSIAIPTNFTISVTTEILPVSMTKTSVDCTMYICGDSTECSNLLLQYGS
FCTQLNRALTGIAVEQDKNTQEVFAQVKQIYKTPPIKDFGGENFSQILPDPSKPSKRSPIED
LLFNKVTLADAGFIKQYGDCLGDIAARDLICAQKENGLTVLPPLLTDEMIAQYTSALLAGTI
TSGWTFGAGPALQIPFPMQMAYRENGIGVTQNVLYENQKLIANQFNSAIGKIQDSLSSTPSA
LGKLQDVVNQNAQALNTLVKQLSSNFGAISSVLNDILSRLDPPEAEVQIDRLITGRLQSLQT
YVTQQLIRAAEIRASANLAATKMSECVLGQSKRVDFCGKGYHLMSFPQSAPHGVVFLHVTYV
PAQEKNFTTAPAICHDGKAHFPREGVFVSNGTHWFVTQRNFYEPQIITTDNTFVSGNCDVVI
GIVNNTVYDPLQPELD

Fusion proteins according to the embodiments of the present invention include an amino acid sequence of a ferritin subunit polypeptide (“ferritin amino acid sequence”). The ferritin amino acid sequence can be an amino acid sequence of a full length, single ferritin polypeptide, or any portion of ferritin amino acid sequence that is capable of directing self-assembly of monomeric ferritin subunits into oligomers. Fusion proteins including ferritin amino acid sequences are described, for example, in U.S. Pat. No. 7,097,841. The amino acid sequences of monomeric ferritin subunits, or portions thereof, of any ferritin protein can be used to produce fusion proteins of the present disclosure, so long as the monomeric ferritin subunits are capable of self-assembling into an oligomer or a nanoparticle. Variations can be made in the amino acid sequence of a ferritin protein without affecting its ability to self-assemble into an oligomer or a nanoparticle. Such variations include insertion of amino acid residues, deletions of amino acid residues, or substitutions of amino acid residues. For example, the sequence of a monomeric ferritin subunit included in a fusion protein according to the embodiments of the present invention can be derived from a mammalian ferritin amino acid sequence, but be divergent enough from the naturally occurring sequence, such that, when administered as an immunogen to a mammalian subject of the species from which the mammalian ferritin amino acid sequence was derived, it does not result in the production of antibodies that react with the natural ferritin protein of the mammal. A ferritin amino acid sequence may be derived from a bacterial ferritin protein, a plant ferritin protein, an algal ferritin protein, an insect ferritin protein, a fungal ferritin protein, and/or a mammalian ferritin protein. In some embodiments of fusion proteins of the present disclosure, ferritin amino acid sequence is derived from H. pylori. For example, a ferritin amino acid sequence included in a fusion protein as provided herein may be or may be derived from a sequence with at least 80%, at least 85%, at least 90%, at least 95%, at least 96%, at least 97%, at least 98%, or at least 99% sequence identity to SEQ ID NO:2. As discussed above, fusion proteins according to the embodiments the present invention need not comprise a full-length sequence of a ferritin subunit polypeptide of H. pylori. Portions, or regions, of H. pylori ferritin subunit polypeptide can be can be used that contain an amino acid sequence directing self-assembly of monomeric ferritin subunits into oligomers. One example of such a region is located between amino acids 5 and 168 of the amino acid sequence H. pylori ferritin protein. More regions are described in Zhang, 2011.

A ferritin amino acid sequence included in fusion proteins according to the embodiments of the present invention may include artificial glycosylation sites, for example, artificial (engineered) N-glycosylation sites, which are engineered by inserting artificial mutations into a ferritin amino acid sequence to create a consensus glycosylation sequence. For example, an artificial N-glycosylation site may be created by introducing a consensus sequence N-X-S/T (where X cannot be P) in a ferritin nucleic acid sequence. A consensus glycosylation sequence can be created by artificial substitutions of amino acid residues in a ferritin amino acid sequence. For example, an artificial N-glycosylation site in SEQ ID NO:2 can be created by introducing two amino acid substitutions: K to N at a position corresponding to position 75 of SEQ ID NO:2, and E to T at a position corresponding to position 75 of SEQ ID NO:2. In another example, an artificial N-glycosylation site in SEQ ID NO:2 can be created by introducing two amino acid substitutions: T to N at a position corresponding to position 67 of SEQ ID NO:2, and I to T at a position corresponding to position 69 of SEQ ID NO:2. In yet another example, an artificial N-glycosylation site in SEQ ID NO:2 can be created by introducing two amino acid substitutions: H to N at a position corresponding to position 74 of SEQ ID NO:2, and F to T at a position corresponding to position 76 of SEQ ID NO:2. In one more example, an artificial N-glycosylation site in SEQ ID NO:2 can be created by introducing two amino acid substitutions: E to N at a position corresponding to position 143 of SEQ ID NO:2, and H to T at a position corresponding to position 145 of SEQ ID NO:2.

Embodiments of fusion proteins according to the present invention include an amino acid sequence of a Spike protein of a coronavirus, such as SARS-CoV-2 (for example, an amino acid sequence with at least 80%, at least 85%, at least 90%, at least 95%, at least 96%, at least 97%, at least 98%, or at least 99% sequence identity to SEQ ID NO:3, SEQ ID NO:4, SEQ ID NO:14, or SEQ ID NO:15) joined to at least 25 contiguous amino acids, at least 50 contiguous amino acids, at least 75 contiguous amino acids, at least 100 contiguous amino acids, or at least 150 contiguous amino acids of an amino acid sequence of a ferritin subunit polypeptide. In the embodiments of fusion proteins according to the present invention, an amino acid sequence of a ferritin subunit polypeptide is positioned after an amino acid sequence of a Spike protein of a coronavirus (i.e. downstream or C′ terminally relative to the Spike protein amino acid sequence). Due to the presence of an amino acid sequence of a ferritin subunit polypeptide, fusion proteins according to the embodiments of the present invention assemble into nanoparticles, which are described in more detail elsewhere in the present disclosure. In some embodiments of a fusion protein, an amino acid sequence of a Spike protein of a coronavirus is joined to at least 25 contiguous amino acids, at least 50 contiguous amino acids, at least 75 contiguous amino acids, at least 100 contiguous amino acids, or at least 150 contiguous amino acids an amino acid sequence of a ferritin subunit polypeptide of H. pylori. An amino acid sequence of a ferritin subunit polypeptide of H. pylori that is included in a fusion protein according to the embodiments of the present invention can have at least 80%, at least 85%, at least 90%, at least 95%, at least 97%, at least 98%, or at least 99% sequence identity to SEQ ID NO:2. An amino acid sequence of a ferritin subunit polypeptide of H. pylori that is included in a fusion protein according to the embodiments of the present invention results in a fusion protein that self-assembles into oligomers or nanoparticles.

In some embodiments of the fusion proteins according to the present invention, an amino an amino acid sequence of a Spike protein of a coronavirus and an amino acid sequence of a ferritin subunit polypeptide are joined by a “linker” amino acid sequence. The peptide linker may be, for example, 2 to 5, 2 to 10, 2 to 20, 2 to 30, 2 to 40, 2 to 50, or 2 to 60, or more amino acids in length, for example, 2 amino acids, 3 amino acids, 4 amino acids, 5 amino acids, 10 amino acids, 15 amino acids, 25 amino acids, 35 amino acids, 45 amino acids, 50 amino acids, or 60 amino acids. Depending on length, linker sequence may have various conformations in secondary structure, such as helical, β-strand, coil/bend, and turns. In some instances, a linker sequence may have an extended conformation and function as an independent domain that does not interact with the adjacent protein domains. A linker sequence may be rigid or flexible. A flexible linker sequence may increase the range of orientations that may be adopted by the domains of the fusion protein. A rigid linker can be used to keep a fixed distance between the domains and to help maintain their independent functions. Linker sequences for fusion proteins are described, for example, in Chen et al., 2013. In some embodiments, a linker is or includes an amino acid sequence SGG, GSG, GG, GSGG (SEQ ID NO:5), NGTGGSG (SEQ ID NO:6), G, or GGGGS (SEQ ID NO:7). In an exemplary embodiment of a fusion protein, a Spike protein amino acid sequence with at least 80%, at least 85%, at least 90%, at least 95%, at least 96%, at least 97%, at least 98%, or at least 99% sequence identity to SEQ ID NO:3 or SEQ ID NO:4 is joined to an amino acid sequence of a ferritin subunit polypeptide with at least 80%, at least 85%, at least 90%, at least 95%, at least 96%, at least 97%, at least 98%, or at least 99% sequence identity to SEQ ID NO:2 by a linker with or including an amino acid sequence SGG, GSG, GG, GSGG (SEQ ID NO:5), NGTGGSG (SEQ ID NO:6), G, or GGGGS (SEQ ID NO:7).

Fusion proteins described in a present disclosure may include a domain or sequence useful for protein isolation. In some embodiments, the polypeptides comprise an affinity tag, for example an AviTag™, a Myc tag, a polyhistidine tag (such as 8×His tag), an albumin-binding protein, an alkaline phosphatase, an AU1 epitope, an AU5 epitope, a biotin-carboxy carrier protein (BCCP), or a FLAG epitope, to name a few. In some embodiments, the affinity tags are useful for protein isolation. See, for example, Kimple et al., 2013. In some embodiments, the polypeptides or proteins include a signal sequence useful for protein isolation, for example a mutated Interleukin-2 signal peptide sequence, which promotes secretion and facilitates protein isolation. See, for example, Low et al., 2013. In some embodiments, a fusion protein may include a protease recognition site, for example, TEV protease cut site, which may be useful for, among other things, removal of a signal peptide or affinity purification tag following fusion protein isolation.

Some embodiments of the fusion proteins described in the present disclosure may include a coronavirus signal sequence, for example, in order to facilitate secretion of fusion proteins from cells after expression. For example, in some embodiments, a coronavirus Spike protein amino acid sequence may be preceded by a native coronavirus signal sequence. In exemplary embodiments, a Spike protein amino acid sequence with at least 80%, at least 85%, at least 90%, at least 95%, at least 96%, at least 97%, at least 98%, or at least 99% sequence identity to SEQ ID NO:3, SEQ ID NO:4, SEQ ID NO:14, or SEQ ID NO:15 is preceded by a native coronavirus signal sequence MFVFLVLLPLVSSQ (SEQ ID NO:8), MFVFLVLLPLVS (SEQ ID NO:31), or MFVFLVLLPLVSS (SEQ ID NO:32), which may be referred to as “signal sequence.” The signal sequence may immediately precede Spike protein amino acid sequence, or can there be a linker or a spacer sequence between the signal sequence and the Spike protein amino acid sequence. Some examples of amino acid sequences of the fusion proteins according to the embodiments of the present invention are sequences with at least 80%, at least 85%, at least 90%, at least 95%, at least 96%, at least 97%, at least 98%, or at least 99% sequence identity to SEQ ID NO:12, SEQ ID NO:13, SEQ ID NO:16, SEQ ID NO:17, SEQ ID NO:18, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:33, or SEQ ID NO:34. Some examples of amino acid sequences of the fusion proteins according to the embodiments of the present invention are sequences with at least 80%, at least 85%, at least 90%, at least 95%, at least 96%, at least 97%, at least 98%, or at least 99% sequence identity to SEQ ID NO:12 without the N-terminal signal sequence (SEQ ID NO:8), SEQ ID NO:13 without the N-terminal signal sequence (SEQ ID NO:8), SEQ ID NO:16 without the N-terminal signal sequence (SEQ ID NO:8), SEQ ID NO:17, SEQ ID NO:18 without the N-terminal signal sequence (SEQ ID NO:8), SEQ ID NO:21 without the N-terminal signal sequence (SEQ ID NO:8), SEQ ID NO:22 without the N-terminal signal sequence (SEQ ID NO:8), SEQ ID NO:23 without the N-terminal signal sequence (SEQ ID NO:8), SEQ ID NO:24 without the N-terminal signal sequence (SEQ ID NO:8), SEQ ID NO:25 without the N-terminal signal sequence (SEQ ID NO:8), SEQ ID NO:26 without the N-terminal signal sequence (SEQ ID NO:8), SEQ ID NO:27 without the N-terminal signal sequence (SEQ ID NO:8), SEQ ID NO:28 without the N-terminal signal sequence (SEQ ID NO:8), SEQ ID NO:29 without the N-terminal signal sequence (SEQ ID NO:8), SEQ ID NO:30 without the N-terminal signal sequence (SEQ ID NO:8), SEQ ID NO:33 without the N-terminal signal sequence (SEQ ID NO:31), or SEQ ID NO:34 without the N-terminal signal sequence (SEQ ID NO:32).

Provided in this disclosure and included among the embodiments of the present invention are nanoparticles that include fusion proteins comprising an amino acid sequence of a Spike protein of a coronavirus and an amino acid sequence of a ferritin subunit polypeptide. Due to the fact that fusion proteins according to the embodiments the present invention include an amino acid sequence of a ferritin subunit polypeptide, they can self-assemble into oligomers. An oligomeric structure, or supramolecule, resulting from such self-assembly is referred to as a as a nanoparticle. An exemplary embodiment of the present invention is a nanoparticle comprising an oligomer of a fusion protein, as described in the present disclosure.

Nanoparticles according to the embodiments of the present invention can contain 24 fusion protein subunits and have 432 symmetry. Nanoparticles according to the embodiments of the present invention display at least a portion of the Spike protein on their surface as trimers. In other words, a nanoparticle according to the embodiments of the present invention comprises surface-exposed trimers of coronavirus Spike protein. A nanoparticle can include eight surface-exposed trimers of coronavirus Spike protein. When the nanoparticle is administered to a subject, the surface-exposed trimers of coronavirus Spike protein trimer are accessible to the immune system of the subject to and thus can elicit an immune response to coronavirus Spike protein. Immunogenic nanoparticles composed of fusion proteins incorporating ferritin amino acid sequences are described, for example, in U.S. Pat. Nos. 9,441,19 and 10,137,190, Kanekiyo et al., 2013, Kanekiyo et al., 2015, and He et al., 2016.

### Nucleic Acids, Vectors, Cells, and Related Methods

Provided in this disclosure and included among the embodiments of the present invention are nucleic acids encoding fusion proteins according to the embodiments of the present invention and described elsewhere in the present disclosure. Nucleic acids according to the embodiments of the present invention encode fusion proteins of an amino acid sequence of a Spike protein of a coronavirus (“coronavirus Spike protein”) and an amino acid sequence of a ferritin subunit polypeptide (which can be referred to simply as “ferritin”). Nucleic acids according to the embodiments of the present invention can be DNA or RNA. Nucleic acids described in the present disclosure can be used for producing fusion proteins and nanoparticles according to the embodiments of the present invention. For example, nucleic acids described in the present disclosure can be used for producing fusion proteins and nanoparticles according to the embodiments of the present invention in order to generate fusion proteins or nanoparticles to be used as immunogenic compositions, or vaccines, against coronaviruses, such as, but not limited to, SARS-CoV-2. In another example, nucleic acids described in the present disclosure can be used as nucleic acid vaccines, which are administered to subjects for the purpose of producing in subject fusion proteins and nanoparticles according to the embodiments of the present invention, in order to elicit in the subjects protective immune response against a coronavirus, including, but not limited to, SARS-CoV-2. Methods of using nucleic acids according to the embodiments of the present invention are described elsewhere in the present disclosure.

Embodiments of nucleic acids encoding fusion proteins described in the present disclosure encode fusion proteins including an amino acid sequence of a Spike protein of a coronavirus, such as SARS-CoV-2 (for example, an amino acid sequence with at least 80%, at least 85%, at least 90%, at least 95%, at least 96%, at least 97%, at least 98%, or at least 99% sequence identity to SEQ ID NO:3, SEQ ID NO:4, SEQ ID NO:14, or SEQ ID NO:15) joined to at least 25 contiguous amino acids, at least 50 contiguous amino acids, at least 75 contiguous amino acids, at least 100 contiguous amino acids, or at least 150 contiguous amino acids an amino acid sequence of a ferritin subunit polypeptide. Some embodiments of nucleic acids encoding fusion proteins described in the present disclosure encode fusion proteins in which an amino acid sequence of a Spike protein of a coronavirus, such as SARS-CoV-2 (for example, an amino acid sequence with at least 80%, at least 85%, at least 90%, at least 95%, at least 96%, at least 97%, at least 98%, or at least 99% sequence identity to SEQ ID NO:3, SEQ ID NO:4, SEQ ID NO:14, or SEQ ID NO:15) is joined to at least 25 contiguous amino acids, at least 50 contiguous amino acids, at least 75 contiguous amino acids, at least 100 contiguous amino acids, or at least 150 contiguous amino acids of a ferritin subunit polypeptide of H. pylori, such as an amino acid sequence with at least 80%, at least 85%, at least 90%, at least 95%, at least 97%, at least 98%, or at least 99% sequence identity to SEQ ID NO:2. Some examples of nucleic acids described in the present disclosure encode fusion proteins having amino acid sequences with at least 80%, at least 85%, at least 90%, at least 95%, at least 96%, at least 97%, at least 98%, or at least 99% sequence identity to SEQ ID NO:12, SEQ ID NO:13, SEQ ID NO:16, SEQ ID NO:17, SEQ ID NO:18, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:12 without the N-terminal signal sequence (SEQ ID NO:8), SEQ ID NO:13 without the N-terminal signal sequence (SEQ ID NO:8), SEQ ID NO:16 without the N-terminal signal sequence (SEQ ID NO:8), SEQ ID NO:17 without the N-terminal signal sequence (SEQ ID NO:8), SEQ ID NO:18 without the N-terminal signal sequence (SEQ ID NO:8), SEQ ID NO:21 without the N-terminal signal sequence (SEQ ID NO:8), SEQ ID NO:22 without the N-terminal signal sequence (SEQ ID NO:8), SEQ ID NO:23 without the N-terminal signal sequence (SEQ ID NO:8), SEQ ID NO:24 without the N-terminal signal sequence (SEQ ID NO:8), SEQ ID NO:25 without the N-terminal signal sequence (SEQ ID NO:8), SEQ ID NO:26 without the N-terminal signal sequence (SEQ ID NO:8), SEQ ID NO:27 without the N-terminal signal sequence (SEQ ID NO:8), SEQ ID NO:28 without the N-terminal signal sequence (SEQ ID NO:8), SEQ ID NO:29 without the N-terminal signal sequence (SEQ ID NO:8), SEQ ID NO:30 without the N-terminal signal sequence (SEQ ID NO:8), SEQ ID NO:33 without the N-terminal signal sequence (SEQ ID NO:31), or SEQ ID NO:34 without the N-terminal signal sequence (SEQ ID NO:32).

Also provided in this disclosure and included among the embodiments of the present invention are nucleic acid constructs that include the nucleic acid sequences provided herein. Some embodiments of the nucleic acid constructs are purified nucleic acid molecules encoding fusion proteins according to the embodiments of the present invention. For example, a nucleic acid construct can be an engineered (recombinant) DNA nucleic acid sequence comprising a promoter operably linked to a nucleic acid encoding a fusion protein according to an embodiment of the present invention. A nucleic acid sequence is “operably linked” when it is placed into a functional relationship with another nucleic acid sequence. A promoter is a region or a sequence located upstream and/or downstream from the start of transcription that is involved in recognition and binding of RNA polymerase and other proteins to initiate transcription. A promoter is generally a nucleic acid sequence or sequences that function when in a relatively fixed location in regard to the transcription start site. A promoter contains core elements required for basic interaction of RNA polymerase and transcription factors, and may contain upstream elements and response elements. A promoter included in nucleic acid constructs according to embodiments of the present invention can be a eukaryotic or a prokaryotic promoter. In some embodiments, the promoter is an inducible promoter. In some embodiments, the promoter is a constitutive promoter. A promoter included in a nucleic acid construct according to the embodiments of the present invention is capable of directing or driving expression of nucleic acid sequence encoding a fusion protein described in the present disclosure in a host cell or host organism of interest. For preparing nucleic acid constructs according to the embodiments of the present invention, nucleic acids may be manipulated, so as to provide for the nucleic acid sequences in the proper orientation and, as appropriate, in the proper reading frame. Toward this end, adapters or linkers may be employed to join the nucleic acid fragments or other manipulations may be involved to provide for convenient restriction sites, removal of superfluous nucleic acid sequences, removal of restriction sites, etc. For this purpose, in vitro mutagenesis, primer repair, restriction, annealing, resubstitutions, such as transitions and transversions, may be involved.

A nucleic acid according to the embodiments of the present invention can be included in an expression cassette for expression of a fusion protein encoded by the nucleic acid in a host cell or an organism of interest. In some embodiments, a nucleic acid according to the embodiments of the present invention can be codon-optimized for expression in a host cell or an organism of interest. An expression cassette can include 5′ and 3′ regulatory sequences operably linked to the nucleic acid encoding a fusion protein according to an embodiment of the present invention. An expression cassette can also include nucleic acid sequences encoding other polypeptides or proteins. An expression cassette can include a plurality of restriction sites and/or recombination sites for insertion of various nucleic acid sequences into the expression cassette and/or for insertion of the expression cassette into other nucleic acids, such as vectors. An expression cassette can include various regulatory regions or sequences, such as, but are not limited to, transcriptional initiation start sites, operators, activators, enhancers, other regulatory elements, ribosomal binding sites, initiation codons, termination signals, and the like. Exemplary regulatory sequences included in the expression cassettes are promoters, transcriptional regulatory regions, and/or translational termination regions, which may be endogenous or heterologous to the host cell or host organism, or to each other. In this context, “heterologous” means a nucleic acid sequence that does not originate in the host cell or host organism, or is substantially modified from its form occurring in the host cell or host organism. An expression cassette can also include one or more selectable marker genes for the selection of host cells containing the expression cassette. Marker genes include, but are not limited to, genes conferring antibiotic resistance, such as those conferring hygromycin resistance, ampicillin resistance, gentamicin resistance, neomycin resistance, to name a few. Additional selectable markers are known and any can be used. An exemplary expression cassette can include, in the 5′ to 3′ direction, a transcriptional and translational initiation region (including a promoter), a nucleic acid sequence encoding a fusion protein described in the present disclosure, and transcriptional and translational termination regions functional in the host cell or host organism of interest.

Also included among the embodiments of the present invention are vectors including nucleic acids or nucleic acid constructs according to the embodiments of the present invention. Such vectors can include necessary functional elements that direct and regulate transcription of the nucleic acid sequences included in the vector. These functional elements include, but are not limited to, a promoter, regions upstream or downstream of the promoter, such as enhancers that may regulate the transcriptional activity of the promoter, an origin of replication, appropriate restriction sites to facilitate cloning of inserts adjacent to the promoter, antibiotic resistance genes or other markers that can serve to select for cells containing the vector or the vector containing the insert, RNA splice junctions, a transcription termination region, or any other region that may serve to facilitate the expression of the inserted gene or hybrid. The vector, for example, can be a plasmid.

A vector according to the embodiments of the present invention can be a bacterial vector, such as a bacterial expression vector. For example, a vector based on one of numerous E. coli expression vectors can be useful for the expression of a nucleic acid according to the embodiments of the present invention. Other bacterial hosts suitable for expression of nucleic acids according to the embodiments of the present invention include bacilli, such as Bacillus subtilis, and other enterobacteriaceae, such as Salmonella, Senatia, and various Pseudomonas species. In these prokaryotic hosts, one can also use suitable expression vectors, which will typically contain expression control sequences compatible with the host cell (such as an origin of replication). Any number of a variety of well-known promoters can be used in bacterial expression vectors, such as a lactose promoter system, a tryptophan (Trp) promoter system, a beta-lactamase promoter system, or a promoter system from phage lambda.

Eukaryotic cells, including, but not limited to, yeast cells, mammalian cells and insect cells, also permit the expression of proteins in an environment that favors important post-translational modifications such as folding and cysteine pairing, addition of complex carbohydrate structures, and secretion of active protein. Accordingly, vectors useful for the expression of nucleic acids described in the present disclosure in yeast cells, mammalian cells and insect cells are also envisioned and included among the embodiments of the present invention. A vector according to the embodiments of the present invention can be a yeast expression vector suitable for expression of a nucleic acid according to the embodiments of the present invention in yeast cells, such as, but not limited to, cells of Pichia pastoris or Saccharomyces cerevisiae. Expression vectors used in eukaryotic cells may contain sequences necessary for the termination of transcription. These regions are transcribed as polyadenylated segments in the untranslated portion of the mRNA. Accordingly, a transcription unit included in an eukaryotic expression vector may contain a polyadenylation region. One benefit of this region is that it increases the likelihood that the transcribed unit will be processed and transported like mRNA. The 3′ untranslated regions also include transcription termination sites. Expression vectors for eukaryotic cells can include expression control sequences, such as enhancers, and necessary information processing sites, such as ribosome binding sites, RNA splice sites etc.

Expression vectors according to the embodiments of the present invention can also include nucleic acids described in the present disclosure under the control of an inducible promoter such as the tetracycline inducible promoter or a glucocorticoid inducible promoter. The nucleic acids of the present invention can also be under the control of a tissue-specific promoter to promote expression of the nucleic acid in specific cells, tissues or organs. Any regulatable promoter, such as a metallothionein promoter, a heat-shock promoter, and other regulatable promoters are also contemplated. Furthermore, a Cre-loxP inducible system can also be used, as well as a Flp recombinase inducible promoter system.

In some embodiments, a nucleic acid encoding a fusion protein according to the embodiments of the present invention may be incorporated into a viral vector for delivery into a host cell or host organism. Accordingly, the vectors according to the embodiments of the present invention include viral vectors that transport the nucleic acids encoding fusion proteins described in the present disclosure into cells without degradation and include a promoter yielding expression of the nucleic acids in the cells into which it is delivered. Suitable viral vectors include adenovirus vectors, adeno-associated viral (AAV) vectors, herpes viral vectors, retroviral vectors, poxviral vectors, or lentiviral vectors. Methods of constructing and using such vectors are well known. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA. The necessary functions of the removed early genes are typically supplied by cell lines that have been engineered to express the gene products of the early genes in trans.

For example, recombinant viruses in the pox family of viruses can be used as vectors for delivering the nucleic acid molecules according to the embodiments of the present invention into a host cell or host organism. These include vaccinia viruses and avian poxviruses, such as the fowlpox and canarypox viruses. Methods for producing recombinant pox viruses are known. Representative examples of recombinant pox viruses include ALVAC, TROVAC, and NYVAC. In another example, adenovirus vectors can be used for delivering the nucleic acid molecules according to the embodiments of the present invention into a host cell or host organism. In one more example, adeno-associated virus (AAV) vector systems can be used for delivering the nucleic acid molecules according to the embodiments of the present invention into a host cell or host organism. In one more example, retroviral vectors can be used for delivering the nucleic acid molecules according to the embodiments of the present invention into a host cell or host organism. Examples of retroviral vectors include, but are not limited to, vectors based on Murine Maloney Leukemia virus (MMLV), and retroviruses that express the desirable properties of MMLV as a vector. In yet another example, molecular conjugate vectors, such as the adenovirus chimeric vectors can be used for delivering nucleic acid molecules according to the embodiments of the present invention into a host cell or host organism. Vectors derived from the members of the Alphavirus genus, such as, but not limited to, Sindbis, Semliki Forest, and Venezuelan Equine Encephalitis viruses, can also be used for delivering nucleic acid molecules according to the embodiments of the present invention into a host cell or host organism.

Also provided in this disclosure and included among the embodiments of the present invention are cells comprising a nucleic acid, a nucleic acid construct, or a vector according to the embodiments of the present invention. Such cells can be referred to as “host cells” (or “host cell,” in singular). Some host cells can produce fusion proteins described in the present disclosure, while other host cells may be used for producing or maintaining nucleic acids, DNA constructs, or vectors according to the embodiments of the present invention. A host cell can be an in vitro, ex vivo, or in vivo host cell. Populations of any of the host cells and cell cultures comprising one or more host cells are also included among the embodiments of the present invention. The host cell can be a prokaryotic cell, including, for example, a bacterial cell. Alternatively, the cell can be a eukaryotic cell. Examples of prokaryotic host cells are cells of E. coli, Pseudomonas, Bacillus or Streptomyces. Examples of eukaryotic cells are yeast cells (such as cells of Saccharomyces yeast, or methylotrophic yeast such as Pichia, Candida, Hansenula, and Torulopsis); animal cells, such as CHO, R1. 1, B-W and LM cells, African Green Monkey kidney cells (for example, COS 1, COS 7, BSC1, BSC40, and BMT10), insect cells (for example, Sf9), human cells (such as human embryonic kidney cells, for instance, HEK293, or HeLa cells).

Methods of producing or generating host cells (meaning cells comprising a nucleic acid, a nucleic acid construct, or a vector according to the embodiments of the present invention) are also included among the embodiments of the present invention. A nucleic acid, a nucleic acid construct, or a vector according to the embodiments of the present invention can be transferred or introduced into the host cell by well-known methods, which vary depending on the type of the host cell. The “introducing” and the related terms or phrases used in the context of introducing a nucleic acid a nucleic acid construct, or a vector into a cell refers to the translocation of the nucleic acid sequence from outside a cell to inside the cell. In some cases, introducing refers to translocation of the nucleic acid from outside the cell to inside the nucleus of a eukaryotic cell. Various methods of such translocation are contemplated, including but not limited to, electroporation, nanoparticle delivery, viral delivery, contact with nanowires or nanotubes, receptor mediated internalization, translocation via cell penetrating peptides, liposome mediated translocation, DEAE dextran, lipofectamine, calcium phosphate or any method now known or identified in the future for introduction of nucleic acids into prokaryotic or eukaryotic cellular hosts. A targeted nuclease system (e.g., an RNA-guided nuclease (CRISPR-Cas9), a transcription activator-like effector nuclease (TALEN), a zinc finger nuclease (ZFN), or a megaTAL (MT) can also be used to introduce a nucleic acid into a cell.

Methods of producing or generating fusion proteins and nanoparticles described in the present disclosure are also included among the embodiments of the present invention. An exemplary method of producing the fusion protein or a nanoparticle can include a step of introducing into a cell a nucleic acid according to an embodiment of the present invention, a nucleic acid construct according to an embodiment of the present invention, or a vector according to an embodiment of the present invention. The introducing step is carried out as described elsewhere in the present disclosure, and, as an outcome of such step, a cell (which can be referred to as “a host cell”) comprising the nucleic acid, the nucleic acid construct or the vector is generated. An exemplary method of producing the fusion protein can include a step of incubating the host cell under conditions allowing for expression of a fusion protein. An exemplary method of producing the nanoparticle can include a step of incubating the host cell under conditions allowing for expression of a fusion protein and self-assembly of the nanoparticle. After expression in the host cell, a fusion protein or a nanoparticles can be isolated or purified using various purification methods. In some embodiments, the fusion protein can be isolated from the host cell and allowed to self-assemble into nanoparticles in vitro.

In one example illustrating a process of producing or generating fusion proteins and nanoparticles described in the present disclosure, a nucleic acid or a nucleic acid construct encoding a fusion protein according to an embodiment of the present invention is introduced into a plasmid or other vector, which is then used to transform living cells. For instance, a nucleic acid encoding a fusion protein according to an embodiment of the present invention is inserted in a correct orientation into an expression vector that provides the necessary regulatory regions, such as promoters, enhancers, poly A sites and other sequences. In some cases. it may be desirable to express the fusion protein under the control of an inducible or tissue-specific promoter. The expression vector may then be transfected into living cells using various methods, such as lipofection or electroporation, thus generating host cells expressing the fusion protein. The cells the fusion protein may be selected by appropriate antibiotic selection or other methods and cultured. Larger amounts of the fusion protein may be produced by growing the cells in commercially available bioreactors. Once expressed by the host cells, the fusion protein may be isolated (purified) according to standard procedures, such as dialysis, filtration and chromatography. A step of lysing the cells to isolate the fusion protein can be included. Thus, a method of producing or generating a fusion protein according to an embodiment of the present invention may contain one or more steps of culturing a cell comprising a vector under conditions permitting expression of the fusion protein, harvesting the cells and/or harvesting the medium from the cultured cells, and isolating the fusion protein from the cells and/or the culture medium. Compositions, methods and kits related to the production of fusion proteins described in the present disclosure are included within the scope of the embodiments of the present invention.

### Immunogenic Compositions and Kits

Immunogenic compositions containing any of the fusion proteins described in the present disclosure, nanoparticle described in the present disclosure, nucleic acids described in the present disclosure, nucleic acids constructs described in the present disclosure, or vectors described in the present disclosure are included among the embodiments of the present invention. Immunogenic compositions according to the embodiments of the present invention can be also referred to as “vaccines.” An immunogenic composition may contain a fusion protein, a nanoparticle, a nucleic acid, a nucleic acids construct, or a vector according to the present invention and a pharmaceutically acceptable carrier (excipient). An immunogenic composition may contain a fusion protein, a nanoparticle, a nucleic acid, a nucleic acids construct, or a vector according to the embodiments of the present invention and an adjuvant. An immunogenic composition contain may contain a fusion protein, a nanoparticle, a nucleic acid, a nucleic acids construct, or a vector according to the embodiments of the present invention and other components, such as, but not limited to, a diluent, solubilizer, emulsifier, or preservative. An immunogenic composition according to the present invention may be a solution, such as an aqueous solution, a suspension, such as an aqueous suspension, or may be in dry form, such as in lyophilized form. Some of the components (or ingredients) included in immunogenic compositions in addition to a fusion protein, a nanoparticle, a nucleic acid, a nucleic acids construct, or a vector according to the embodiments of the present invention are described in more detail elsewhere in the present disclosure.

Some embodiments of the immunogenic compositions contain one or more fusion proteins or nucleic acids encoding the fusion proteins described elsewhere in the present disclosure. For example, an immunogenic composition may contain two or more, three or more, four or more, five or more etc. different fusion proteins described elsewhere in the present disclosure. In another example, an immunogenic composition may contain nucleic acids encoding two or more, three or more, four or more, five or more etc. different fusion proteins described elsewhere in the present disclosure. The nucleic acids encoding two or more, three or more, four or more, five or more etc. different fusion may be included in the same nucleic acid construct, such as a vector, or in different nucleic acid constructs. For example, an immunogenic composition can contain one or more, two or more, three or more, four or more, five or more etc. of fusion proteins or nucleic acids encoding fusion proteins having amino acid sequences that have at least 80%, at least 85%, at least 90%, at least 95%, at least 96%, at least 97%, at least 98%, or at least 99% sequence identity to SEQ ID NO:12 without the N-terminal signal sequence (SEQ ID NO:8), SEQ ID NO:13 without the N-terminal signal sequence (SEQ ID NO:8), SEQ ID NO:16 without the N-terminal signal sequence (SEQ ID NO:8), SEQ ID NO:17, SEQ ID NO:18 without the N-terminal signal sequence (SEQ ID NO:8), SEQ ID NO:21 without the N-terminal signal sequence (SEQ ID NO:8), SEQ ID NO:22 without the N-terminal signal sequence (SEQ ID NO:8), SEQ ID NO:23 without the N-terminal signal sequence (SEQ ID NO:8), SEQ ID NO:24 without the N-terminal signal sequence (SEQ ID NO:8), SEQ ID NO:25 without the N-terminal signal sequence (SEQ ID NO:8), SEQ ID NO:26 without the N-terminal signal sequence (SEQ ID NO:8), SEQ ID NO:27 without the N-terminal signal sequence (SEQ ID NO:8), SEQ ID NO:28 without the N-terminal signal sequence (SEQ ID NO:8), SEQ ID NO:29 without the N-terminal signal sequence (SEQ ID NO:8), SEQ ID NO:30 without the N-terminal signal sequence (SEQ ID NO:8), SEQ ID NO:33 without the N-terminal signal sequence (SEQ ID NO:31), or SEQ ID NO:34 without the N-terminal signal sequence (SEQ ID NO:32).

An immunogenic composition according to the embodiments of the present invention can include a pharmaceutically acceptable carrier or excipient. A pharmaceutically acceptable carrier or excipient is a material that is not biologically or otherwise undesirable, meaning the material that can be administered to a subject without causing undesirable biological effects or interacting in a deleterious manner with the other components of the pharmaceutical composition in which it is contained. The carrier or excipient is typically selected to minimize degradation of other ingredients of the composition in which the carrier or the excipient is included, and to minimize adverse side effects (such as allergic side effects) in the subject. Examples of aqueous pharmaceutically acceptable carriers include, but are not limited to, sterile water, saline, buffered solutions like Ringer's solution, glycerol solutions, ethanol, dextrose solutions, allantoic fluid, or combinations of the foregoing. The pH of the aqueous carriers is generally about 5 to about 8 or from about 7 to 7.5. A carrier may include a pH controlling buffer. The preparation of such aqueous carriers insuring sterility, pH, isotonicity, and stability is effected according to established protocols. Examples of non-aqueous carriers are propylene glycol, polyethylene glycol, vegetable oils such as olive oil, and injectable organic esters such as ethyl oleate. Other exemplary carries sustained release preparations, such as semipermeable matrices of solid hydrophobic polymers. Other exemplary carriers are matrices in the form of shaped articles, such as, but not limited to, films, liposomes, or microparticles. Certain carriers may be more preferable depending upon, for instance, the route of administration and concentration of composition being administered.

An immunogenic composition according to the embodiments of the present invention can include an adjuvant. Some examples of chemical adjuvants are aluminum phosphate, benzyalkonium chloride, ubenimex, QS21, aluminium hydroxide (such as alum, an aluminum hydroxide wet gel suspension, for example, Alhydrogel® (Croda International, UK)), saponins (for example, Quil-A® (Croda International, UK)), squalenes (for example, AddaVax™). Some examples of the so-called “genetic” adjuvants are IL-2 gene or its fragments, granulocyte macrophage colony-stimulating factor (GM-CSF) gene or fragments thereof, IL-18 gene or fragments thereof, chemokine (C-C motif) ligand 21 (CCL21) gene or fragments thereof, IL-6 gene or fragments thereof, CpG, LPS, TLR agonists (for example, Monophosphoryl Lipid A (MPLA)), and other immune stimulatory genes. Some examples of protein adjuvants are IL-2 or fragments thereof, granulocyte macrophage colony-stimulating factor (GM-CSF) or fragments thereof, IL-18 or its fragments, chemokine (C-C motif) ligand 21 (CCL21) or fragments thereof, IL-6 or fragments thereof, CpG, LPS, TLR agonists and other immune stimulatory cytokines or their fragments. Some examples of lipid adjuvants are cationic liposomes, N3 (cationic lipid), MPLA, Quil-A®, and AddaVax™ Other exemplary adjuvants include, but are not limited to, cholera toxin, enterotoxin, Fms-like tyrosine kinase-3 ligand (Flt-3L), bupivacaine, marcaine, and levamisole. In some embodiments, the immunogenic composition comprises Quil-A®. In some embodiments, the immunogenic composition comprises alum. In some embodiments, the immunogenic composition comprises CpG. More than one adjuvant may be included in immunogenic compositions according to the embodiments of the present invention. For example, in some embodiments, the immunogenic composition can comprise alum and CpG.

Immunogenic compositions according to the embodiments of the present invention are generally formulated to be nontoxic or minimally toxic to subject at the dosages and concentrations used for administration. In some embodiments, a formulation of an immunogenic compositions may include an appropriate amount of a pharmaceutically acceptable salt to render the formulation isotonic. In some embodiments, a formulation of an immunogenic compositions may include components for modifying, maintaining, or preserving, for example, the pH, osmolality, viscosity, clarity, color, isotonicity, odor, sterility, stability, rate of dissolution or release, adsorption or penetration of the composition. A formulation of an immunogenic composition may include one or more of the following components: amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides, disaccharides, and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); and/or delivery vehicles.

In some embodiments, an immunogenic composition can be prepared in a dry form (i.e. dehydrated form), such as a lyophilized form. Such a formulation can be referred to as “lyophilized” or a “lyophilizate.” Lyophilization is a process of or freeze-drying, during which a solvent is removed from a liquid formulation. Lyophilization process may include one or more of simultaneous or sequential steps of freezing and drying. Immunogenic compositions according to the embodiments of the present invention can be lyophilized in an aqueous solution comprising a nonvolatile or volatile buffer. Non-limiting examples of suitable nonovolatile buffers are PBS, Tris-HCl, HEPES, or L-Histidine buffer. Non-limiting examples of suitable volatile buffers are ammonium bicarbonate, Ammonia/acetic acid, or N-ethylmorpholine/acetate buffer. A lyophilized immunogenic composition according to the embodiments of the present invention can include appropriate carriers or excipients. Such appropriate excipients may include, but are not limited to, a cryo-preservative, a bulking agent, a surfactant, or their combinations. Exemplary excipients include one or more of a polyol, a disaccharide, or a polysaccharide, such as, for example, mannitol, sorbitol, sucrose, trehalose, and/or dextran 40. In some instances, the cryo-preservative may be sucrose and/or trehalose. In some instances, the bulking agent may be glycine or mannitol. In one example, the surfactant may be a polysorbate such as, for example, polysorbate-20 and/or polysorbate-80. A lyophilized immunogenic composition according to the embodiments of the present invention can be, for example, in a cake or powder form. Lyophilized immunogenic compositions may be rehydrated/solubilized/reconstituted in a carrier or excipient (e.g., water or buffer solution) prior to use. Some embodiments of the immunogenic compositions are reconstituted in a water or buffer solution comprising sucrose.

An immunogenic composition according to embodiments of the present invention can be sterile prior to administration to a subject. Sterilization can be accomplished by filtration through sterile filtration membranes. When the immunogenic composition is lyophilized, sterilization can be conducted either prior to or following lyophilization and reconstitution. An immunogenic composition can be stored in sterile containers, such as vials or bags, as a solution, suspension, gel, emulsion, solid, or as a dehydrated or lyophilized powder.

Kits including immunogenic compositions described in the present disclosure are also included among the embodiments of the present invention. For example, a kit may include an immunogenic composition and a container for its storage, such as a bag or a vial. Such a container may have a sterile access port, for example, a bag or vial having a stopper pierceable by a hypodermic injection needle. In another example, a kit may include an immunogenic composition in lyophilized or concentrated form and diluent. In such a kit, a diluent may also be a pharmaceutically acceptable carrier or excipient, as described elsewhere in the present disclosure. Examples of diluents that may be included in such a kit are saline, buffered saline, water, or sucrose. In another example, a kit may include an immunogenic composition and a device for administering the immunogenic composition. A device for administering the composition may be a syringe for injection or oral administration (for example, the kit may be a syringe pre-filled with a liquid immunogenic composition), a microneedle device, such as a microneedle patch, an inhaler, or a nebulizer. In some embodiments, a kit may contain a defined amount of an immunogenic composition capable of eliciting a protective immune response against a coronavirus in a subject, when administered as a single dose. In some embodiments, a kit may contain multiple doses of a defined amount of an immunogenic composition capable of eliciting a protective immune response against a coronavirus in a subject. For example, a kit may contain multiple vials, syringes or microneedle patches containing an immunogenic composition.

### Methods of Inducing an Immune Response

Methods of inducing or eliciting an immune response against a coronavirus in a subject by administering to the subject the an immunogenic composition described in the present disclosure are included among the embodiments of the present invention. In embodiments of such methods, an immunogenic composition is administered in an amount capable of inducing or eliciting a protective immune response against a coronavirus in the subject. A protective immune response against a coronavirus in the subject may include production of anti-coronavirus neutralizing antibodies in the subject. An amount of the immunogenic composition capable of inducing or eliciting a protective immune response against a coronavirus in the subject can be described as an “effective amount” or “immunologically effective amount,” and may be administered as one dose or as two or more doses. Effective amounts and schedules for administration may be determined empirically.

Dosage ranges for administration of the immunogenic compositions described in the present disclosure are those large enough to produce the desired effect—i.e. eliciting a protective immune response against a coronavirus, such as SARS-CoV-2. The dosage should not be so large as to cause substantial adverse side effects, such as unwanted cross-reactions, anaphylactic reactions, and the like. Generally, the dosage may vary with the age, condition, sex, medical status, route of administration, or whether other drugs are included in the regimen. The dosage can be adjusted by a medical professional in the event of any contraindications. Dosages can vary, and the agent can be administered in one or more dose administrations daily, for one or several days, including a prime and boost paradigm.

When used in the context of methods of inducing or eliciting a protective immune response against a coronavirus in a subject, immunogenic compositions described in the present disclosure can be administered via any of several routes of administration, including, but not limited to, orally, parenterally, intravenously, intramuscularly, subcutaneously, transdermally, by nebulization/inhalation, or by installation via bronchoscopy. An immunogenic composition can be administered by oral inhalation, nasal inhalation, or intranasal mucosal administration. Administration of the immunogenic compositions described in the present disclosure by inhalant can be through the nose or mouth via delivery by spraying or droplet mechanism, for example, in the form of an aerosol. A form of administration may be chosen to optimize a protective immune response against a coronavirus in a subject.

In the provided methods in which the immunogenic composition comprises a nucleic acid, a nucleic acid construct, or a vector according to the embodiments of the present invention (such a composition may be termed a “nucleic acid immunogenic composition” or a “nucleic acid vaccine”), the immunogenic composition can be introduced into the cells of the subject. Examples of nucleic acid delivery technologies include “naked DNA” facilitated (bupivacaine, polymers, peptide-mediated) delivery, and cationic lipid complexes or liposomes. The nucleic acids can be administered using ballistic delivery as described, for instance, in U.S. Pat. No. 5,204,253 or pressure (see, for example, U.S. Pat. No. 5,922,687). In some examples, particles comprised solely or mostly of a nucleic acid, a nucleic acid construct, or a vector according to the embodiments of the present invention can be administered to the subject. In some examples, a nucleic acid, a nucleic acid construct, or a vector according to the embodiments of the present invention can be adhered to particles, such as gold particles, for administration to the subject. When an immunogenic composition includes a viral vector, the viral vector can be introduced into cells obtained from the subject (autologous cells) and the cells can be administered to the subject. In some embodiments, an immunogenic composition comprising a nucleic acid, a nucleic acid construct, or a vector according to the embodiments of the present invention can be administered by injection or electroporation, or a combination of injection and electroporation.

In the context of the methods described in the present disclosure, a subject may be healthy and without higher risk for a coronavirus invention than the general public. In some instances, the subject can have an elevated risk of developing a coronavirus infection such that they are predisposed to contracting an infection, or may be predisposed to developing a serious form of coronavirus disease, such as COVID-19 (for example, persons over 65, persons with asthma or other chronic respiratory disease, young children, pregnant women, persons with a hereditary predisposition, persons with a compromised immune system may be predisposed to developing a serious form of COVID-19). A subject may also be a subject with a current coronavirus infection, and may have one or more than one symptom of the infection. A subject currently with a coronavirus infection may have been diagnosed with coronavirus infection based on the symptoms or the results of diagnostic test.

The methods according to the embodiments of the present invention are useful for both prophylactic and therapeutic purposes. Methods of treating or preventing a coronavirus infection in a subject, which include administering to a subject with coronavirus infection or susceptible to a coronavirus infection an effective dose an immunogenic compositions described in the present disclosure are also included among the embodiments of the present invention. In the methods according to the embodiments of the present invention, an immunogenic composition can be used alone or in combination with one or more therapeutic agents such as, for example, antiviral compounds for the treatment of coronavirus infection or disease. For prophylactic use, an effective amount of an immunogenic compositions described in the present disclosure can be administered to a subject prior to onset of coronavirus infection (for example, before obvious signs of infection) or during early onset (for example, upon initial signs and symptoms of infection). Prophylactic administration can occur at several days to years prior to the manifestation of symptoms of coronavirus infection. Prophylactic administration can be used, for example, in the preventative treatment of subjects identified as having a predisposition to a coronavirus infection. Therapeutic treatment involves administering to a subject a therapeutically effective amount of an immunogenic composition described in the present disclosure after diagnosis or development of infection.

In the context of the embodiments of the present invention, the terms “treatment,” “treat,” “treating” and the related terms and expressions refer to reducing one or more of the effects of a coronavirus infection or one or more symptoms of the coronavirus infection by eliciting an immune response in the subject. Thus in the disclosed method, treatment can refer to a 10%, 20%, 30%, 40%, 50%, 60%, 70%, 80%, 90%, or 100% reduction in the severity of an established coronavirus infection or a symptom of the coronavirus infection. For example, a method for treating a coronavirus infection is considered to be a treatment if there is a 10% reduction in one or more symptoms of the coronavirus infection in a subject, as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the coronavirus infection or disease or symptoms of the coronavirus infection or disease.

In the context of the embodiments of the present invention, the terms “prevent,” “preventing,” “prevention” of a coronavirus infection or disease, and the related terms and expressions, refer to an action, for example, administration of an immunogenic composition that occurs before or at about the same time a subject begins to show one or more symptoms of the coronavirus infection, which inhibits or delays onset or exacerbation or delays recurrence of one or more symptoms of the infection. As used in the present disclosure, references to decreasing, reducing, or inhibiting include a change of 10%, 20%, 30%, 40%, 50%, 60%, 70%, 80%, 90% or greater as compared to a control level. For example, the methods described in the present disclose can be considered to effect prevention of a coronavirus infection, if there is about a 10% reduction in onset, exacerbation or recurrence of a coronavirus infection, or symptoms of infection in a subject exposed to a coronavirus to whom an immunogenic composition described in the present disclosure was administered, when compared to control subjects exposed to coronavirus that did not receive a composition for decreasing infection. Thus, the reduction in onset, exacerbation or recurrence of a coronavirus infection can be about a 10, 20, 30, 40, 50, 60, 70, 80, 90, 100%, or any amount of reduction in between as compared to control subjects.

### EXAMPLES

The following examples are offered to illustrate, but not to limit the claimed invention.

### Example 1: Materials and Methods

A. DNA Constructs.

The construct encoding receptor binding domain (RBD) of SARS-CoV-2 Spike protein (“RBD construct”) is described in Amanat et al. (2020). The SARS-CoV-2 Spike receptor RBD spans amino acid residues 319-541 of SARS-CoV-2 Wuhan-Hu-1. The RBD construct contains nucleic acid sequence encoding the native signal peptide (amino acids 1-14), followed by the sequence encoding residues 319-541 from the SARS-CoV-2 Wuhan-Hu-1 genome sequence (GenBank Ref No. MN9089473), and a sequence encoding hexahistidine tag at the C-terminus.

Full-length and C-terminally truncated (ΔC) SARS-CoV-2 Spike protein ectodomain constructs were prepared from full-length Spike protein construct also described in Amanat et al. (2020), which contains a nucleic acid sequence from the SARS-CoV-2 Wuhan-Hu-1 genome sequence (GenBank MN9089473) encoding residues 1-1213 of the Spike protein, with the furin site (RRAR) mutated to alanine, and two proline mutations (K986P and V987P) stabilizing the Spike trimer in the prefusion conformation. Following the nucleic acid sequence encoding residue 1213 of the Spike protein, nucleic acid sequences were added encoding a GCN4 trimerization domain and hexahisitine tag. The above construct (“FL Spike trimer”) was used as a basis for the construct encoding truncated SARS-CoV-2 Spike protein ectodomain with the deletion of heptad repeat 2 (HR2). The construct encoding AC SARS-CoV-2 Spike protein ectodomain (“SpikeΔC trimer”), only the sequence encoding residues 1-1137 of the Spike protein was included. The above constructs were transferred into pADD2 mammalian expression vector using HiFi PCR (Takara), followed by InFusion cloning with EcoRI/XhoI restriction sites. Full-length Spike ferritin (“FL Spike ferritin”) and AC Spike ferritin (“SpikeΔC ferritin”) constructs were cloned by PCR-amplifying the sequences encoding either full-length Spike protein ectodomain (residues 1-1213) or AC Spike protein ectodomain (residues 1-1143) off the expression vector, followed by stitching PCR, in which the constructs were annealed to an amplicon encoding SGG linker followed by H. pylori ferritin sequence (residues 5-168). The resulting amplicons were then inserted into the pADD2 mammalian expression vector via InFusion, using EcoRI/XhoI restriction sites. The final sequences were confirmed using Sanger Sequencing.

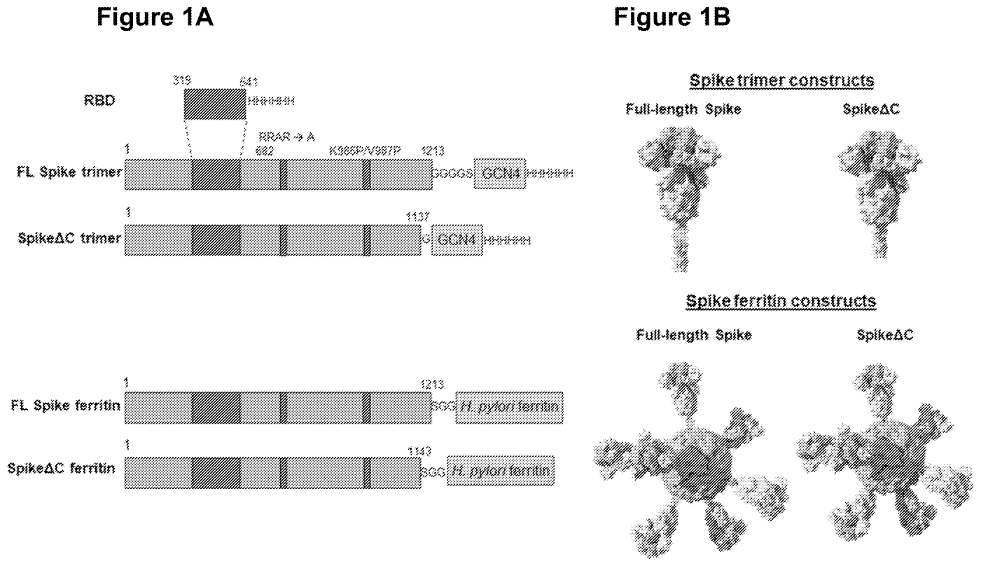

The constructs discussed above are schematically illustrated in FIG. 1, and the amino acid sequences encoded by the constructs are shown below as SEQ ID NOs 7-11, with SARS-CoV-2 Spike signal peptide sequence shown in bold/underlined font, Hexahistidine tag sequences shown in bold, Ser/Gly linker regions underlined, GCN4 trimerization domain italicized, and H. pylori ferritin sequences italicized and underlined.

RBD-
SEQ ID NO: 9
MFVFLVLLPLVSSQRVQPTESIVRFPNITNLCPFGEVENATRFASVYAWNRKRISNCVADYS
VLYNSASFSTFKCYGVSPTKLNDLCFTNVYADSFVIRGDEVRQIAPGQTGKIADYNYKLPDD
FTGCVIAWNSNNLDSKVGGNYNYLYRLFRKSNLKPFERDISTEIYQAGSTPCNGVEGENCYF
PLQSYGFQPTNGVGYQPYRVVVLSFELLHAPATVCGPKKSTNLVKNKCVNFHHHHHH
FL Spike trimer-
SEQ ID NO: 10
MFVFLVLLPLVSSQCVNLTTRTQLPPAYTNSFTRGVYYPDKVFRSSVLHSTQDLFLPFFSNV
TWFHAIHVSGTNGTKRFDNPVLPFNDGVYFASTEKSNIIRGWIFGTTLDSKTQSLLIVNNAT
NVVIKVCEFQFCNDPFLGVYYHKNNKSWMESEFRVYSSANNCTFEYVSQPFLMDLEGKQGNF
KNLREFVFKNIDGYFKIYSKHTPINLVRDLPQGFSALEPLVDLPIGINITRFQTLLALHRSY
LTPGDSSSGWTAGAAAYYVGYLQPRTFLLKYNENGTITDAVDCALDPLSETKCTLKSFTVEK
GIYQTSNFRVQPTESIVRFPNITNLCPFGEVENATRFASVYAWNRKRISNCVADYSVLYNSA
SFSTFKCYGVSPTKLNDLCFTNVYADSFVIRGDEVRQIAPGQTGKIADYNYKLPDDFTGCVI
AWNSNNLDSKVGGNYNYLYRLERKSNLKPFERDISTEIYQAGSTPCNGVEGENCYFPLQSYG
FQPTNGVGYQPYRVVVLSFELLHAPATVCGPKKSTNLVKNKCVNFNFNGLTGTGVLTESNKK
FLPFQQFGRDIADTTDAVRDPQTLEILDITPCSFGGVSVITPGTNTSNQVAVLYQDVNCTEV
PVAIHADQLTPTWRVYSTGSNVFQTRAGCLIGAEHVNNSYECDIPIGAGICASYQTQTNSPA
SVASQSIIAYTMSLGAENSVAYSNNSIAIPTNFTISVTTEILPVSMTKTSVDCTMYICGDST
ECSNLLLQYGSFCTQLNRALTGIAVEQDKNTQEVFAQVKQIYKTPPIKDFGGFNFSQILPDP
SKPSKRSFIEDLLENKVTLADAGFIKQYGDCLGDIAARDLICAQKFNGLTVLPPLLTDEMIA
QYTSALLAGTITSGWTFGAGAALQIPFAMQMAYRENGIGVTQNVLYENQKLIANQFNSAIGK
IQDSLSSTASALGKLQDVVNQNAQALNTLVKQLSSNFGAISSVLNDILSRLDPPEAEVQIDR
LITGRLQSLQTYVTQQLIRAAEIRASANLAATKMSECVLGQSKRVDFCGKGYHLMSFPQSAP
HGVVFLHVTYVPAQEKNFTTAPAICHDGKAHFPREGVFVSNGTHWFVTQRNFYEPQIITTDN
TFVSGNCDVVIGIVNNTVYDPLQPELDSFKEELDKYFKNHTSPDVDLGDISGINASVVNIQK
EIDRLNEVAKNLNESLIDLQELGKYEQYIKWPSGRGGGGSRMKQIEDKIEEILSKQYHIENE
IARIKKLIGERGGSGGHHHHHH
ΔC Spike trimer (“SpikeΔC trimer”)-
SEQ ID NO: 11
MFVFLVLLPLVSSQCVNLTTRTQLPPAYTNSFTRGVYYPDKVFRSSVLHSTQDLFLPFFSNV
TWFHAIHVSGTNGTKRFDNPVLPFNDGVYFASTEKSNIIRGWIFGTTLDSKTQSLLIVNNAT
NVVIKVCEFQFCNDPFLGVYYHKNNKSWMESEFRVYSSANNCTFEYVSQPFLMDLEGKQGNF
KNLREFVFKNIDGYFKIYSKHTPINLVRDLPQGFSALEPLVDLPIGINITRFQTLLALHRSY
LTPGDSSSGWTAGAAAYYVGYLQPRTFLLKYNENGTITDAVDCALDPLSETKCTLKSFTVEK
GIYQTSNFRVQPTESIVRFPNITNLCPFGEVENATRFASVYAWNRKRISNCVADYSVLYNSA
SFSTFKCYGVSPTKLNDLCFTNVYADSFVIRGDEVRQIAPGQTGKIADYNYKLPDDFTGCVI
AWNSNNLDSKVGGNYNYLYRLERKSNLKPFERDISTEIYQAGSTPCNGVEGENCYFPLQSYG
FQPTNGVGYQPYRVVVLSFELLHAPATVCGPKKSTNLVKNKCVNFNENGLTGTGVLTESNKK
FLPFQQFGRDIADTTDAVRDPQTLEILDITPCSFGGVSVITPGTNTSNQVAVLYQDVNCTEV
PVAIHADQLTPTWRVYSTGSNVFQTRAGCLIGAEHVNNSYECDIPIGAGICASYQTQTNSPA
SVASQSIIAYTMSLGAENSVAYSNNSIAIPTNFTISVTTEILPVSMTKTSVDCTMYICGDST
ECSNLLLQYGSFCTQLNRALTGIAVEQDKNTQEVFAQVKQIYKTPPIKDFGGFNFSQILPDP
SKPSKRSFIEDLLENKVTLADAGFIKQYGDCLGDIAARDLICAQKENGLTVLPPLLTDEMIA
QYTSALLAGTITSGWTFGAGAALQIPFAMQMAYRENGIGVTQNVLYENQKLIANQFNSAIGK
IQDSLSSTASALGKLQDVVNQNAQALNTLVKQLSSNFGAISSVLNDILSRLDPPEAEVQIDR
LITGRLQSLQTYVTQQLIRAAEIRASANLAATKMSECVLGQSKRVDFCGKGYHLMSFPQSAP
HGVVFLHVTYVPAQEKNFTTAPAICHDGKAHFPREGVFVSNGTHWFVTQRNFYEPQIITTDN
TFVSGNCDVVIGIVNNTVYDPGRMKQIEDKIEEILSKQYHIENEIARIKKLIGERGGSGGHH
HHHH
FL Spike ferritin fusion protein (“FL Spike ferritin”)-
SEQ ID NO: 12
MFVFLVLLPLVSSQCVNLTTRTQLPPAYTNSFTRGVYYPDKVFRSSVLHSTQDLFLPFFSNV
TWFHAIHVSGTNGTKRFDNPVLPENDGVYFASTEKSNIIRGWIFGTTLDSKTQSLLIVNNAT
NVVIKVCEFQFCNDPFLGVYYHKNNKSWMESEFRVYSSANNCTFEYVSQPFLMDLEGKQGNE
KNLREFVFKNIDGYFKIYSKHTPINLVRDLPQGFSALEPLVDLPIGINITRFQTLLALHRSY
LTPGDSSSGWTAGAAAYYVGYLQPRTFLLKYNENGTITDAVDCALDPLSETKCTLKSFTVEK
GIYQTSNFRVQPTESIVRFPNITNLCPFGEVENATRFASVYAWNRKRISNCVADYSVLYNSA
SFSTFKCYGVSPTKLNDLCFTNVYADSFVIRGDEVRQIAPGQTGKIADYNYKLPDDFTGCVI
AWNSNNLDSKVGGNYNYLYRLFRKSNLKPFERDISTEIYQAGSTPCNGVEGENCYFPLQSYG
FQPTNGVGYQPYRVVVLSFELLHAPATVCGPKKSTNLVKNKCVNFNFNGLTGTGVLTESNKK
FLPFQQFGRDIADTTDAVRDPQTLEILDITPCSFGGVSVITPGTNTSNQVAVLYQDVNCTEV
PVAIHADQLTPTWRVYSTGSNVFQTRAGCLIGAEHVNNSYECDIPIGAGICASYQTQTNSPA
SVASQSIIAYTMSLGAENSVAYSNNSIAIPTNFTISVTTEILPVSMTKTSVDCTMYICGDST
ECSNLLLQYGSFCTQLNRALTGIAVEQDKNTQEVFAQVKQIYKTPPIKDFGGFNFSQILPDP
SKPSKRSFIEDLLFNKVTLADAGFIKQYGDCLGDIAARDLICAQKENGLTVLPPLLTDEMIA
QYTSALLAGTITSGWTFGAGAALQIPFAMQMAYRENGIGVTQNVLYENQKLIANQFNSAIGK
IQDSLSSTASALGKLQDVVNQNAQALNTLVKQLSSNFGAISSVLNDILSRLDPPEAEVQIDR
LITGRLQSLQTYVTQQLIRAAEIRASANLAATKMSECVLGQSKRVDFCGKGYHLMSFPQSAP
HGVVFLHVTYVPAQEKNFTTAPAICHDGKAHFPREGVFVSNGTHWFVTQRNFYEPQIITTDN
TFVSGNCDVVIGIVNNTVYDPLQPELDSFKEELDKYFKNHTSPDVDLGDISGINASVVNIQK
EIDRLNEVAKNLNESLIDLQELGKYEQYIKWPSGRSGGDIIKLLNEQVNKEMQSSNLYMSMS
SWCYTHSLDGAGLFLFDHAAEEYEHAKKLIIFLNENNVPVQLTSISAPEHKFEGLTQIFQKA
YEHEQHISESINNIVDHAIKSKDHATENFLQWYVAEQHEEEVLFKDILDKIELIGNENHGLY
LADQYVKGIAKSRKS
ΔC Spike ferritin fusion protein (“SpikeΔC ferritin”)-
SEQ ID NO: 13
MFVFLVLLPLVSSQCVNLTTRTQLPPAYTNSFTRGVYYPDKVFRSSVLHSTQDLFLPFFSNV
TWFHAIHVSGTNGTKRFDNPVLPFNDGVYFASTEKSNIIRGWIFGTTLDSKTQSLLIVNNAT
NVVIKVCEFQFCNDPFLGVYYHKNNKSWMESEFRVYSSANNCTFEYVSQPFLMDLEGKQGNE
KNLREFVFKNIDGYFKIYSKHTPINLVRDLPQGFSALEPLVDLPIGINITRFQTLLALHRSY
LTPGDSSSGWTAGAAAYYVGYLQPRTELLKYNENGTITDAVDCALDPLSETKCTLKSFTVEK
GIYQTSNERVQPTESIVRFPNITNLCPFGEVENATRFASVYAWNRKRISNCVADYSVLYNSA
SFSTFKCYGVSPTKLNDLCFTNVYADSFVIRGDEVRQIAPGQTGKIADYNYKLPDDFTGCVI
AWNSNNLDSKVGGNYNYLYRLFRKSNLKPFERDISTEIYQAGSTPCNGVEGENCYFPLQSYG
FQPTNGVGYQPYRVVVLSFELLHAPATVCGPKKSTNLVKNKCVNFNFNGLTGTGVLTESNKK
FLPFQQFGRDIADTTDAVRDPQTLEILDITPCSFGGVSVITPGTNTSNQVAVLYQDVNCTEV
PVAIHADQLTPTWRVYSTGSNVFQTRAGCLIGAEHVNNSYECDIPIGAGICASYQTQTNSPA
SVASQSIIAYTMSLGAENSVAYSNNSIAIPTNFTISVTTEILPVSMTKTSVDCTMYICGDST
ECSNLLLQYGSFCTQLNRALTGIAVEQDKNTQEVFAQVKQIYKTPPIKDFGGFNFSQILPDP
SKPSKRSFIEDLLFNKVTLADAGFIKQYGDCLGDIAARDLICAQKENGLTVLPPLLTDEMIA
QYTSALLAGTITSGWTFGAGAALQIPFAMQMAYRENGIGVTQNVLYENQKLIANQFNSAIGK
IQDSLSSTASALGKLQDVVNQNAQALNTLVKQLSSNFGAISSVLNDILSRLDPPEAEVQIDR
LITGRLQSLQTYVTQQLIRAAEIRASANLAATKMSECVLGQSKRVDFCGKGYHLMSFPQSAP
HGVVFLHVTYVPAQEKNFTTAPAICHDGKAHFPREGVFVSNGTHWFVTQRNFYEPQIITTDN
TFVSGNCDVVIGIVNNTVYDPLQPELDSGGDIIKLLNEQVNKEMQSSNLYMSMSSWCYTHSL
DGAGLFLFDHAAEEYEHAKKLIIFLNENNVPVQLTSISAPEHKFEGLTQIFQKAYEHEQHIS
ESINNIVDHAIKSKDHATFNFLQWYVAEQHEEEVLFKDILDKIELIGNENHGLYLADQYVKG
IAKSRKS

The variable heavy chain and variable light chain sequences for SARS-CoV-2 reactive monoclonal antibodies, CR3022, CB6, and COVA-2-15 were codon-optimized for human expression and ordered as gene block fragments from Integrated DNA Technologies (IDT). Fragments were PCR-amplified and inserted into linearized CMV/R expression vectors containing either the heavy chain or light chain Fc sequence from VRC01 using InFusion.

Soluble human ACE2 fused to an Fc tag was constructed by PCR amplifying ACE2 (residues 1-615) from an Addgene plasmid and fusing it to a human Fc domain, separated by a TEV-GSGG (SEQ ID NO:12) linker using a stitching PCR step. hACE2-Fc was then inserted into pADD2 mammalian expression vector via the InFusion® cloning system using EcoRI/XhoI cut sites.

All cloned plasmids were sequence-confirmed using Sanger sequencing. Following sequencing confirmation, plasmids were transformed into Stellar Cells (Takara) and grown overnight in LB/Carbenicillin cultures, with the exception of the CMV/R mAb plasmids which were grown in LB/Kanamycin cultures. Plasmids were prepared for mammalian cell transfection using Macherey Nagel Maxi Prep columns. Eluted DNA was filtered in a biosafety hood using a 0.22 μm filter prior to transfection.

B. Expression and Purification of SARS-CoV-2 Antigens.

All proteins were expressed in Expi293F cells. Expi293F cells were cultured using 66% Freestyle/33% Expi media (ThermoFisher) and grown in TriForest polycarbonate baffled shaking flasks at 37° C. in 8% CO2. The cells were transfected at a density of approximately 3-4×10⁶ cells/mL. Transfection mixtures were made by adding 568 μg maxi-prepped DNA to 113 mL culture media (per liter of transfected cells) followed by addition of 1.48 mL FectoPro (Polyplus). The mixtures were incubated are room temperature for 10 min and then added to cells. Cells were immediately boosted with D-glucose (0.04 g/L final concentration) and 2-propylpentanoic (valproic) acid (3 mM final concentration). The cells were harvested 3-5 days post-transfection by spinning the cultures at 7,000×g for 15 minutes. Supernatants were filtered using a 0.22 μm filter.

RBD, FL Spike trimer, and AC Spike trimer polypeptide antigens were purified using HisPur™ Ni-NTA resin (ThermoFisher). Prior to purification, the resin was washed 3 times with approx. 10 column volumes of wash buffer (10 mM imidazole/1X PBS). Cell supernatants were diluted 1:1 with 10 mM imidazole/1X PBS, the resin was added to diluted cell supernatants, which were then incubated at 4° C. while spinning. Resin/supernatant mixtures were added to glass chromatography columns for gravity flow purification. The resin in the column was washed with 10 mM imidazole/1X PBS, and the proteins were eluted with 250 mM imidazole/1X PBS. Column elutions were concentrated using centrifugal concentrators (10 kDa cutoff for RBD, and 100 kDa cutoff for trimer constructs), followed by size-exclusion chromatography on a AKTA Pure system (Cytiva). RBD was purified using an S200. FL Spike trimer and AC Spike trimer antigens were purified on an S6. Columns were pre-equilibrated in 1×PBS prior to purification.

FL Spike ferritin and AC Spike ferritin nanoparticles were isolated using anion exchange chromatography, followed by size-exclusion chromatography using an SRT® SEC-1000 column. Briefly, Expi293F supernatants were concentrated using a AKTA Flux S column (Cytiva). The buffer was then changed to 20 mM Tris, pH 8.0 via overnight dialysis at 4° C. using 100 kDa molecular weight cut-off (MWCO) dialysis tubing. Dialyzed supernatants were filtered through a 0.22 μm filter and loaded onto a HiTrap® Q anion exchange column equilibrated in 20 mM Tris, pH 8.0. Spike nanoparticles were eluted using a 0-1 M NaCl gradient. Protein-containing fractions were initially identified using Western blot analysis with CR3022, as discussed further below. Protein-containing fractions were pooled and concentrated using a 100 kDa MWCO Amicon® spin filter, and subsequently purified on a AKTA Pure system (Cytiva) using an SRT® SEC-1000 SEC column equilibrated in 1×PBS. Fractions were pooled based on A280 signals and SDS-PAGE analysis on 4-20% Mini-PROTEAN® TGX™ protein gels stained with GelCode™ Blue Stain Reagent (ThermoFisher). Prior to immunizations, the samples were supplemented with 10% glycerol, filtered through a 0.22 μm filter, snap frozen, and stored at −20° C. until use.

C. Western Blot Analysis of Expi Supernatants.

Expi293F supernatants were collected 3 days post-transfection, harvested by spinning at 7,000 xg for 15 minutes, and filtered through a 0.22 μm filter. Samples were diluted in SDS-PAGE Laemmli loading buffer (Bio-Rad), boiled at 95° C., and run on a 4-20% Mini-PROTEAN® TGX protein gel (Bio-Rad) at 250V. Proteins were transferred to nitrocellulose membranes using a Trans-Blot® Turbo™ transfer system (Bio-Rad). Blots were blocked in 5% milk/PBST and following blocking blots were washed with PBST. In-house made primary antibody (CR3022, 5 μM stock concentration) was added at a 1:10,000 in PBST. The blots were washed with PBST and secondary rabbit anti-human IgG H&L HRP (abcam ab6759) was added at 1:50,000 dilution in PBST. The blots were developed using Pierce™ ECL Western blotting substrate (ThermoFisher) and imaged using a GE Healthcare Life Sciences imager.

D. Enzyme-Linked Immunosorbent Assays (ELISAs) with Purified mAbs and Mouse Sera.

ELISA binding with SARS-CoV-2 antigens was performed by coating antigens on MaxiSorp™ 96-well plates (ThermoFisher) at 2 μg/mL in 1×PBS overnight at 4° C. Following coating, the plates were washed 3X with PBST and blocked overnight at 4° C. using ChonBlock™ Blocking/Dilution ELISA Buffer (Chondrex). The buffer was removed manually and plates were washed 3X with PBST. Mouse serum samples, purified monoclonal antibodies, and hACE2-Fc were serially diluted in diluent buffer starting at either 1:50 serum dilution or 10 μg/mL, and then added to coated plates for 1 hr at room temperature. Plates were washed 3X with PBST. For mouse serum ELISAs, HRP goat anti-mouse (BioLegend 405306) was added at a 1:10,000 dilution in diluent buffer for 1 hr at room temperature. For purified mAbs and hACE2-Fc, Direct-Blot HRP anti-human IgG1 Fc antibody was added at a 1:10,000 dilution in diluent buffer for 1 hr at room temperature. Following incubation with secondary antibody, ELISA plates were washed 6X with PBST. Plates were developed for six minutes using 1-Step™ Turbo TMB substrate (Pierce) and were quenched with 2M sulfuric acid. Absorbance at 450 nm was read out using a BioTek plate reader.

E. Mouse Immunizations.

Balb/C mice were procured from The Jackson Laboratories (Bar Harbor, ME). All animals were maintained at Stanford University according to Public Health Service Policy for ‘Humane Care and Use of Laboratory Animals’ following a protocol approved by Stanford University Administrative Panel on Laboratory Animal Care (APLAC). Six to eight weeks old female Balb/C mice were immunized by subcutaneous injection of 10 μg of SARS-Cov-2 Spike protein immunogens (or otherwise stated) with 10 μg Quil-A® adjuvant (InVivogen, San Diego, CA) and 10 μg Monophosphoryl Lipid A (InVivogen, San Diego, CA) (MPLA) as adjuvants diluted in 1×PBS. The list of immunogens and adjuvant combinations is provided in Table 2.

TABLE 2
Immunogens and adjuvant combinations
used in mice immunizations.

| Antigen | Dose | Adjuvant dose |
|---|---|---|
| SARS-CoV-2 RBD | 10 μg | 10 μg Quil-A ®/ |
| | | 10 μg MPLA |
| FL Spike trimer | 10 μg | 10 μg Quil-A ®/ |
| | (monomer concentration) | 10 μg MPLA |
| SpikeΔC trimer | 10 μg | 10 μg Quil-A ®/ |
| | (monomer concentration) | 10 μg MPLA |
| FL Spike ferritin | 10 μg | 10 μg Quil-A ®/ |
| | (monomer concentration) | 10 μg MPLA |
| Spike ΔC ferritin | 10 μg | 10 μg Quil-A ®/ |
| | (monomer concentration) | 10 μg MPLA |

F. SARS-CoV-2 Pseudotyped Lentivirus Production and Viral Neutralization Assays.

SARS-CoV-2 Spike pseudotyped lentivirus was produced in HEK293T cells using calcium phosphate transfection reagent. Six million cells were seeded in D10 media (DMEM+additives: 10% FBS, L-glutamate, penicillin, streptomycin, and 10 mM HEPES) in 10 cm plates one day prior to transfection. A five-plasmid system was used for viral production, including the lentiviral packaging vector (pHAGE_Luc2_IRES_ZsGreen), the SARS-CoV-2 Spike vector (“FL Spike”), and the lentiviral helper plasmids (HDM-Hgpm2, HDM-Tatlb, and pRC-CMV_Revlb), as described in Crawford et al., 2020. The Spike vector contained the full-length wild-type Spike sequence from the Wuhan-Hu-1 strain of SARS-CoV-2. The plasmids were added to filter-sterilized water in the following ratios: 10 μg pHAGE_Luc2_IRS_ZsGreen, 3.4 μg FL Spike, 2.2 μg HDM-Hgpm2, 2.2 μg HDM-Tatlb, 2.2 μg pRC-CMV_Revlb in a final volume of 500 μL. HEPES Buffered Saline (2X, pH 7.0) was added dropwise to this mixture to a final volume of 1 mL. To form transfection complexes, 100 μL 2.5 M CaCl₂) was added dropwise while gently agitating the solution. Transfection reactions were incubated for 20 min at RT, and then slowly added dropwise to plated cells. Culture medium was removed 24 hours post-transfection and replaced with fresh D10 medium. Viral supernatants were harvested 72 hours post-transfection by spinning at 300×g for 5 min followed by filtering through a 0.45 μm filter. Viral stocks were aliquoted and stored at −80° C. until further use.

The target cells used for infection in viral neutralization assays were from a HeLa cell line stably overexpressing the SARS-CoV-2 receptor, ACE2. Production of this cell line is described in detail in Rogers et al., 2020. ACE2/HeLa cells were plated one day prior to infection at 5,000 cells per well. Mouse serum was heat inactivated for 30 min at 56° C., diluted in D10 medium, and incubated with virus for 1 hour at 37° C. Polybrene was added at a final concentration of 5 μg/mL prior to inhibitor/virus dilutions. Following incubation, the medium was removed from the cells, replaced with an equivalent volume of inhibitor/virus dilutions and incubated at 37° C. for approximately 48 hours. Infectivity readout was performed by measuring luciferase levels. Cells were lysed by adding BriteLite™ assay readout solution (Perkin Elmer) and luminescence values were measured using a BioTek plate reader. Each plate was normalized by averaging six cells only (0% infectivity) and six virus only (100% infectivity) wells. Normalized values were fit with a three parameter non-linear regression inhibitor curve in Prism to obtain IC50 values.

G. Cryo-EM Data Acquisition

The samples were diluted to a final concentration of around 0.4 mg/mL for both the AC Spike and FL Spike ferritin nanoparticles, following purification. Three μL of each of the samples were applied onto glow-discharged 200-mesh R2/1 Quantifoil® grids coated with continuous carbon. The grids were blotted for 2 s and rapidly cryocooled in liquid ethane using a Vitrobot™ Mark IV (Thermo Fisher Scientific) at 4° C. and 100% humidity. The samples were screened using a Talos™ Arctica™ cryo-electron microscope (Thermo Fisher Scientific) operated at 200 kV. Then the samples were imaged in a Titan Krios™ cryo-electron microscope (Thermo Fisher Scientific) operated at 300 kV with GIF energy filter (Gatan) at a magnification of 130,000x (corresponding to a calibrated sampling of 1.06 Å per pixel) for both samples. Micrographs were recorded by EPU software (Thermo Fisher Scientific) with a Gatan K2 Summit® direct electron detector, where each image was composed of 30 individual frames with an exposure time of 6 s and an exposure rate of 7.8 electrons per second per Å². A total of 3,684 movie stacks were collected.

H. Single-Particle Image Processing and 3D Reconstruction

All the movie stacks were first imported into RELION (for REgularised LIkelihood OptimisatioN) software for image processing. The motion-correction was performed using MotionCor2, and the contrast transfer function (CTF) was determined using CTFFIND4 (Rohou et al., 2015). All the particles were autopicked using the NeuralNet option in EMAN2, yielding 152,734 particles from selected 3,540 micrographs. Then, particle coordinates were imported to the RELION software, where the poor 2D class averages were removed by several rounds of 2D classification. The initial model was built in the cryoSPARC platform using the ab-initio reconstruction option with octahedral symmetry applied. The final 3D refinement was performed using 62,837 particles with or without octahedral symmetry applied, and a X-A map and a X-A map were obtained, respectively. Resolution for the final maps was estimated with the 0.143 criterion of the Fourier shell correlation curve. A Gaussian low-pass filter was applied to the final 3D maps displayed in the University of California San Francisco Chimera software package.

### Example 2: Expression and Characterization of SARS-CoV-2 Antigens

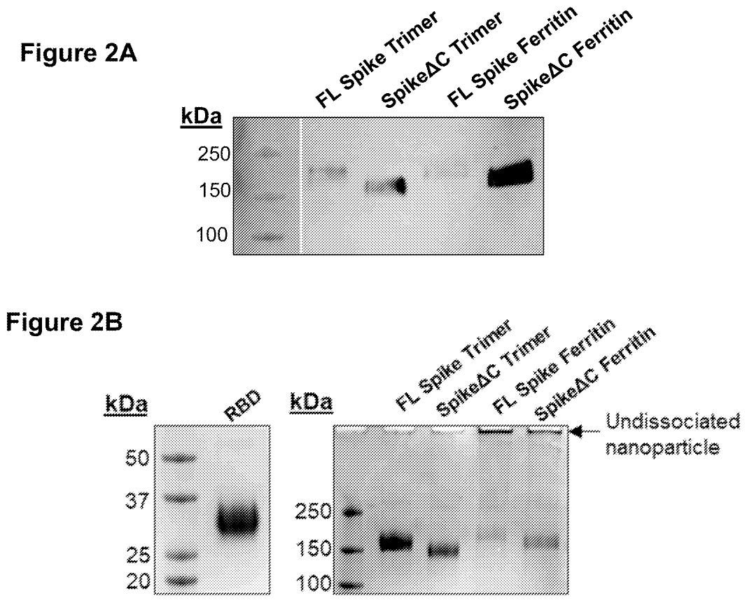

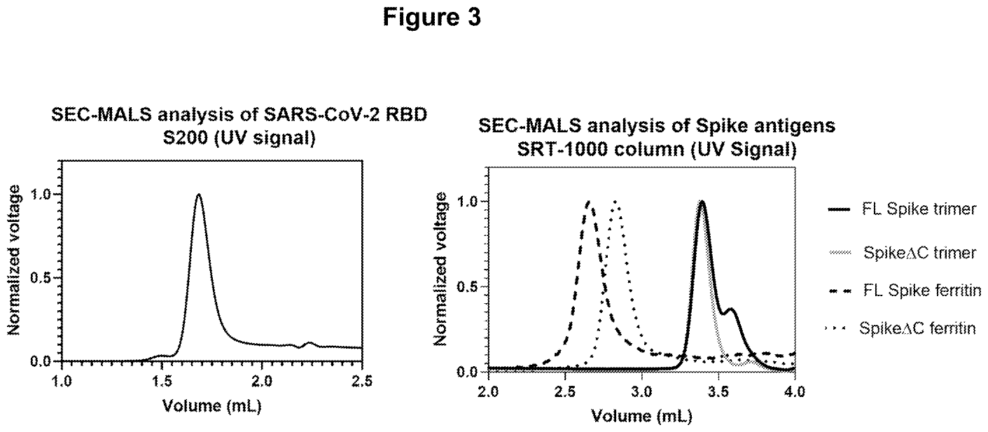

SARS-CoV-2 Spike protein antigens encoded by the constructs described in Example 1 were expressed as discussed in Example 1 and characterized. The results of the characterization are illustrated in FIGS. 2A, 2B and 3. As illustrated in FIG. 2A, Western blot analysis of Expi293F cell supernatant indicated that expression levels varied among different SARS-CoV-2 Spike protein antigens. To produce Western blots shown in FIG. 2A, supernatants were boiled in non-reducing SDS loading buffer, run on a 10% gel for separation, transferred to a nitrocellulose membrane, and blotted with recombinant anti-SARS-CoV-2 Spike Glycoprotein S1 monoclonal antibody (mAb) produced in-house. As illustrated in FIG. 2B, SDS-PAGE analysis of purified SARS-CoV-2 RBD (expected MW 25.9 kDa), FL Spike trimer (expected monomer MW 138.3 kDa), AC Spike trimer (expected monomer MW 129.3 kDa), FL Spike ferritin (expected monomer MW 151.9 kDa), and AC Spike ferritin (expected monomer MW 143.8 kDa) showed as-expected molecular weights of the above SARS-CoV-2 antigens. For SDS-PAGE, the samples were boiled in non-reducing SDS loading buffer, run on a 10% gel for separation, and visualized by Coomassie stain. Analytical scale size-exclusion chromatography coupled with multi-angle light scattering (SEC-MALS) analysis was used to confirm the purity, homogeneity, and size of SARS-CoV-2 antigen preparations prior to immunization of the experimental animals. The results of SEC-MALS analysis are illustrated in FIG. 3. The RBD antigen was analyzed on an S200 column, and the other four antigens were analyzed on an SRT-1000 column. Compiled UV signal, light scattering signal, and refractive index signal from samples were used to calculate an estimated molecular weight and hydrodynamic radius for each preparation using ASTRA software analysis. Importantly, this analysis confirmed that all SARS-CoV-2 Spike protein antigens were stably multimerized and were not dissociating in the monomeric forms. Using the UV, light scattering, and refractive index measurements for each purified protein, we calculated an estimated molecule weight and hydrodynamic radius for each antigen. Additionally, this analysis confirmed that the purified samples were homogenous in nature and were not prone to aggregation under these conditions. The assessment of expression levels from Expi supernatants via a Western blot using CR3022, a SARS1 monoclonal antibody that binds to the SARS-CoV-2 RBD, demonstrated that the C-terminal deletion encompassing the HR2 region resulted in enhancement of expression level in the context of the Spike trimer, and an even greater enhancement in expression of the Spike ferritin fusion protein.

### Example 3: ELISA Binding Analysis of SARS-CoV-2 Spike Protein Antigens

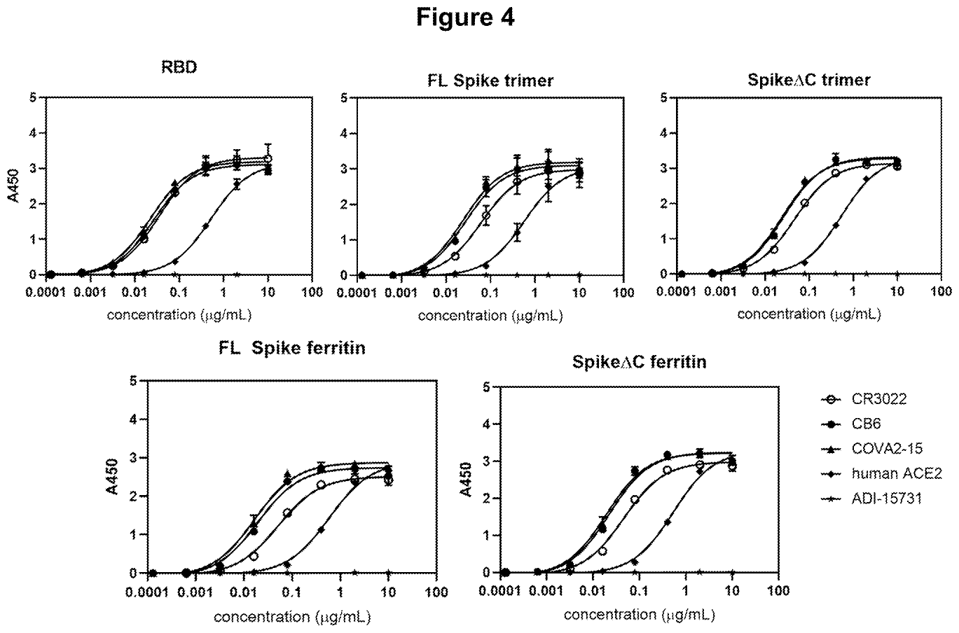

ELISA was used to compare the binding of SARS-CoV-2 Spike protein antigens to human ACE2, COVID-19 purified monoclonal antibodies (CR3022, CB6, COVA2-15), and COVID-19 patient serum (ADI-15731). For ELISA, each SARS-CoV-2 Spike protein antigens were hydrophobically plated at equivalent concentrations. ELISA binding curves illustrated in FIG. 4 indicated that SARS-CoV-2 Spike protein antigens presented both the ACE2 binding site and monoclonal antibody epitopes similarly, as determined by comparable binding levels to each one.

### Example 4: Cryo-EM Analysis SARS-CoV-2 Spike-Ferritin Proteins

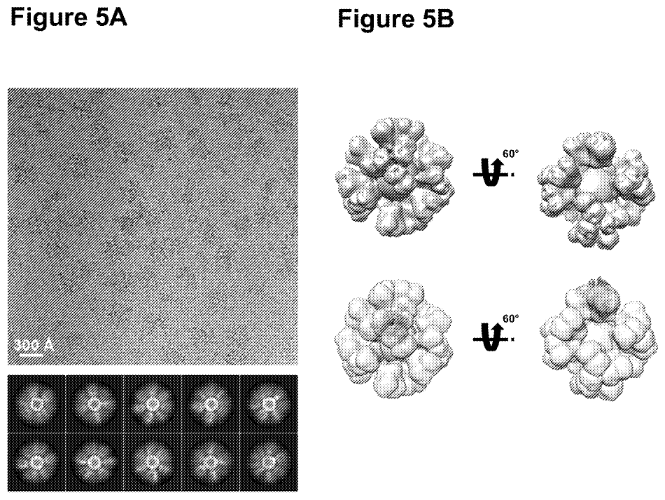

Cryo-EM analysis SARS-CoV-2 Spike-ferritin proteins was performed, with the results illustrated in FIG. 5. Based on the results of Cryo-EM analysis, SARS-CoV-2 Spike-ferritin proteins formed nanoparticles contained of the surface-exposed trimers of the Spike protein of the coronavirus. The cryo-EM raw images of both the FL Spike ferritin and AC Spike ferritin showed clear densities around apoferritin particles, indicating proper formation of the nanoparticles and display of the Spike trimers on the surface. The 2D class averages further showed the densities of the Spike trimers outside the apoferritin, however, the spike protein densities are smeared due to its flexibility. As the raw image and 2D class averages of the AC Spike ferritin particles were better than those of the FL Spike ferritin particles, the former were chose for further data collection and image processing. Using single-particle analysis, the three-dimensional (3D) structure of the AC Spike ferritin complex was determined with and without octahedral symmetry applied. The two cryo-EM maps were very similar, with the cross-correlation coefficient of 0.9857. The cryo-EM analysis confirmed that the Spike trimers were presented in a folded conformation on the surface of the nanoparticles.

### Example 5: Immunogenicity of SARS-CoV-2 Spike Protein Antigens

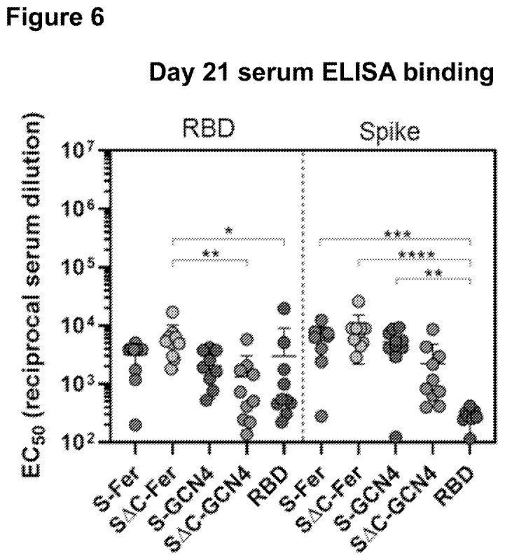

Immunogenicity analysis of SARS-CoV-2 Spike protein antigens was performed, with the experimental results illustrated in FIGS. 6-9. Groups of mice were immunized with 10 μg of each SARS-CoV-2 Spike protein antigen, 10 μg Quil-A® and 10 μg MPLA as adjuvants, with the initial immunization performed at “Day 0.” The mice were bled at “Day 21” and “Day 28” after the initial immunization, and administered a boost dose of immunogen at “Day 21.” The sera extracted from the immunized mice at Day 21 and Day 28 was analyzed by ELISA and luciferase-based SARS-CoV-2 Spike pseudotyped lentiviral assay. Neutralization with pseudotyped viruses is a common way to assess viral inhibition in a research laboratory setting.

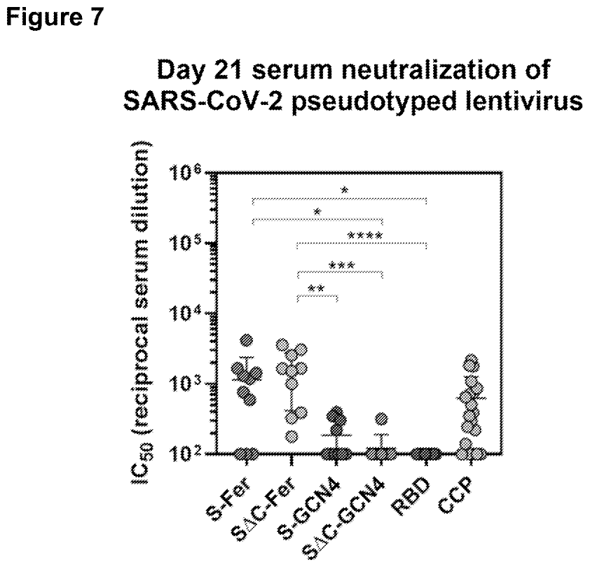

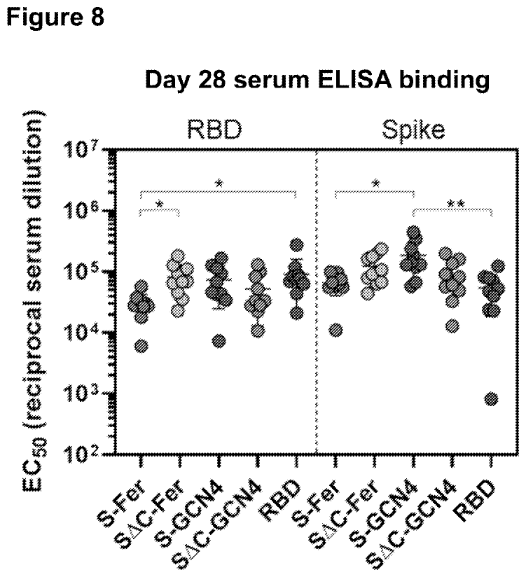

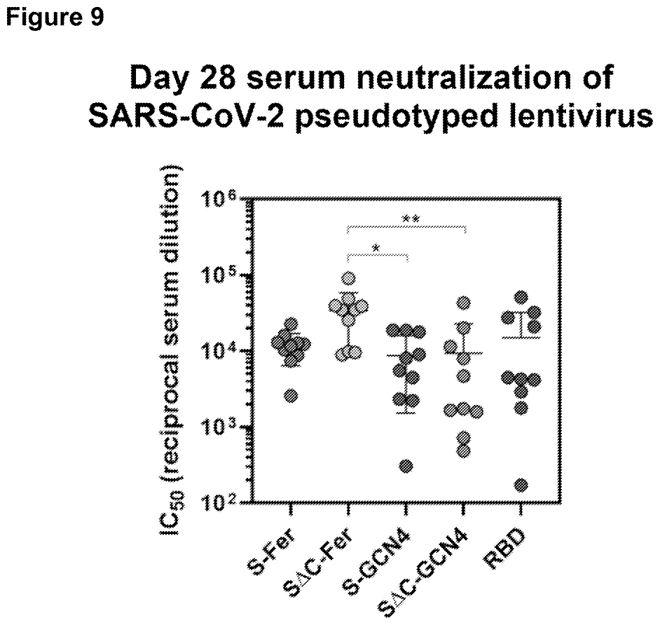

ELISA was used to assess the binding of the sera to SARS-CoV-2 RBD protein and SARS-CoV-2 Spike protein. ELISA binding analysis of the sera extracted at Day 21 (FIG. 6) and Day 28 (FIG. 8) indicated that all five SARS-CoV-2 Spike protein antigens elicited antibodies directed toward the SARS-CoV-2 RBD and full-length Spike proteins. Serum neutralization of SARS-CoV-2 was assessed using a luciferase-based SARS-CoV-2 Spike pseudotyped lentiviral assay. The results of the SARS-CoV-2 Spike pseudotyped lentiviral assay of the sera extracted at Day 21 (FIG. 7) and Day 28 (FIG. 9) indicated that each of SARS-CoV-2 antigens elicited Spike-directed antibodies capable of neutralizing SARS-CoV-2 pseudotyped lentivirus. However, AC Spike ferritin fusion protein elicited the highest neutralizing antibody response in the experimental animals among all the antigens tested. SARS-CoV-2 Spike pseudotyped lentiviral assay was performed on the sera extracted at Day 21, a set of 20 convalescent COVID-19 patient plasma samples (“convalescent COVID-19 plasma,” indicated as “CCP” in FIG. 7) was used for comparison. The comparison indicated that immunization with AC Spike ferritin fusion protein elicited at least two-fold greater neutralizing antibody titers, as compared to convalescent COVID-19 plasma.

### Example 6: Immunoglobulin-Specific Responses Following Immunization with SARS-CoV-2 Spike Protein Antigens

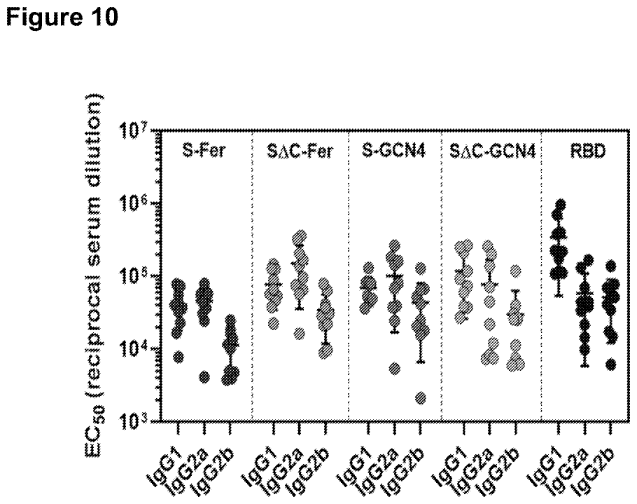

Immunoglobulin-specific responses in the experimental animals (mice) following immunization with SARS-CoV-2 Spike protein antigens adjuvanted with Quil-A®/MPLA were assessed using ELISA. The experimental results are illustrated in FIGS. 10-12. FIG. 10 illustrates the results of ELISA binding analysis of IgG1, IgG2a, and IgG2b subclass responses of the sera extracted from experimental mice immunized with two doses of SARS-CoV-2 Spike protein antigens FL Spike ferritin (“S-Fer”), SpikeΔC ferritin (“SAC-Fer”), FL Spike trimer (“S-GCN4”), SpikeΔC trimer (“SAC-GCN4”), and RBD. Two 10 μg doses of the antigens were administered, with the second dose administered at day 21 after the first administration. The experiments showed that immunization with two doses SARS-CoV-2 Spike protein antigens adjuvanted with Quil-A® and MPLA led to robust IgG1 and IgG2 responses, and minimal levels of IgM responses.

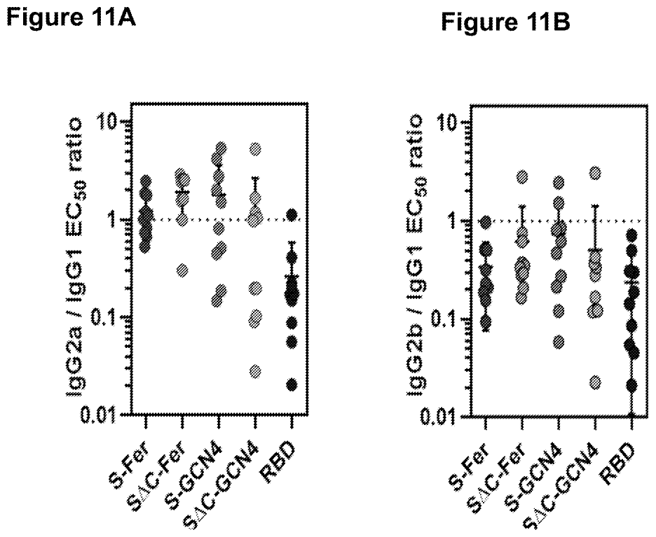

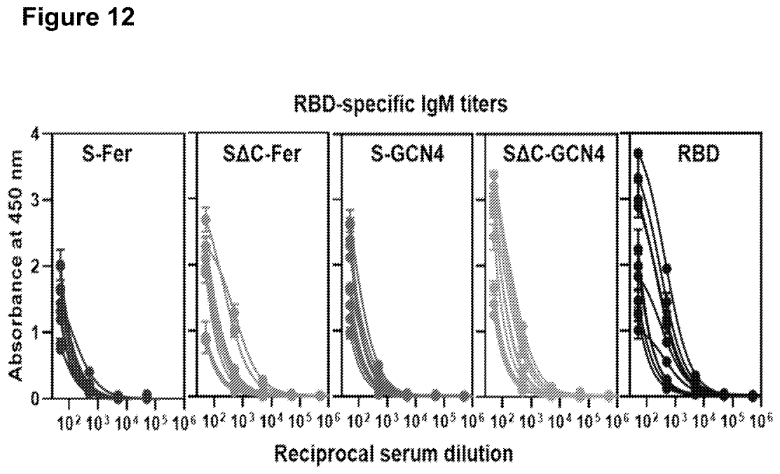

The experimental results illustrated in FIG. 10 demonstrated broad IgG responses with varied ratios of IgG subclasses among different SARS-CoV-2 Spike protein antigen groups. As further illustrated in FIG. 11A, SpikeΔC ferritin and FL Spike trimer elicited higher IgG2a responses, as compared to IgG1 responses, FL Spike ferritin and SpikeΔC trimer groups elicited roughly balanced levels of IgG2a and IgG1 responses, and RBD elicited substantially greater IgG1 response than IgG2a response. As further illustrated in FIG. 11B, each of SARS-CoV-2 Spike protein antigens elicited the responses with IgG2b/IgG1 ratios less than 1, indicating a lower IgG2b response, as compared to IgG1 response. ELISA was also used to determine SARS-CoV-2 Spike protein antigen-specific IgM titers in the experimental animals, with the results illustrated in FIG. 12. Lower levels of IgM, as compared to IgGs, were detected.

### Example 7: Stable Neutralizing Antibody Responses Following Immunization with SARS-CoV-2 Spike Protein Antigens

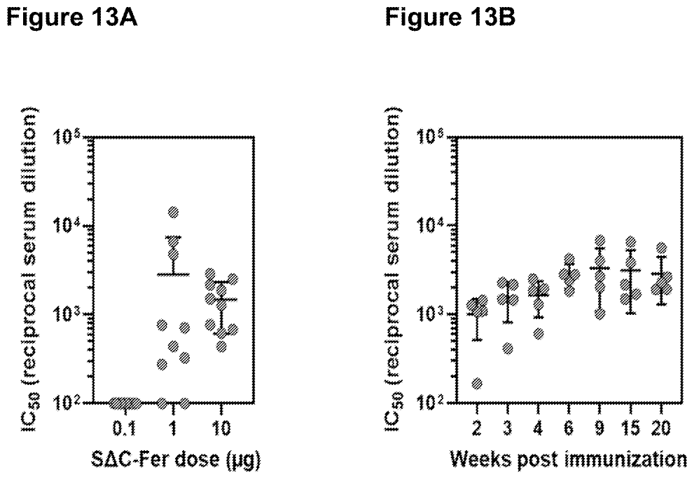

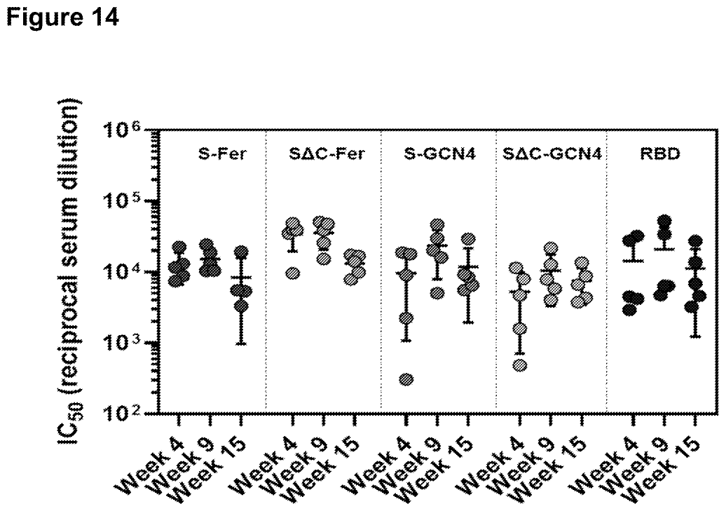

Neutralizing antibody responses following immunization with SARS-CoV-2 Spike protein antigens FL Spike ferritin (“S-Fer”), SpikeΔC ferritin (“SAC-Fer”), FL Spike trimer (“S-GCN4”), SpikeΔC trimer (“SAC-GCN4”), and RBD were assessed using luciferase-based SARS-CoV-2 Spike pseudotyped lentiviral assay, with the results illustrated in FIGS. 13A, 13B and 14. Among other things, the experimental results indicated that immunization with SpikeΔC ferritin led to a dose-dependent neutralizing antibody response and elicited neutralizing antibody levels that were stable up to 20-weeks post immunization.

FIG. 13A illustrates the neutralization properties of the sera extracted from the experimental mice at day 28 after subcutaneous administration of 0.1 μg, 1 μg, or 10 μg SpikeΔC ferritin adjuvanted with 10 μg Quil-A® and 10 μg MPLA. FIG. 13B illustrates that neutralizing antibody responses increased in the experimental animals between 2- and 6-weeks after subcutaneous administration of 20 μg SpikeΔC ferritin adjuvanted with 10 μg Quil-A® and that the neutralizing antibody responses remained stable for up to 20 weeks after SpikeΔC ferritin administration. FIG. 14 illustrates the longevity of neutralizing antibody responses to SARS-CoV-2 Spike protein antigens in the experimental mice following subcutaneous administration of two 10 μg doses of a SARS-CoV-2 Spike protein antigen adjuvanted with 10 μg Quil-A® and 10 μg MPLA in a total volume of 100 μL. The second dose was administered at day 21 after the administration of the first dose. The neutralizing antibody levels were assessed from serum collected at weeks 4, 9, and 15 after the initial administration.

### Example 8: Screening of Adjuvants and Dosing Conditions

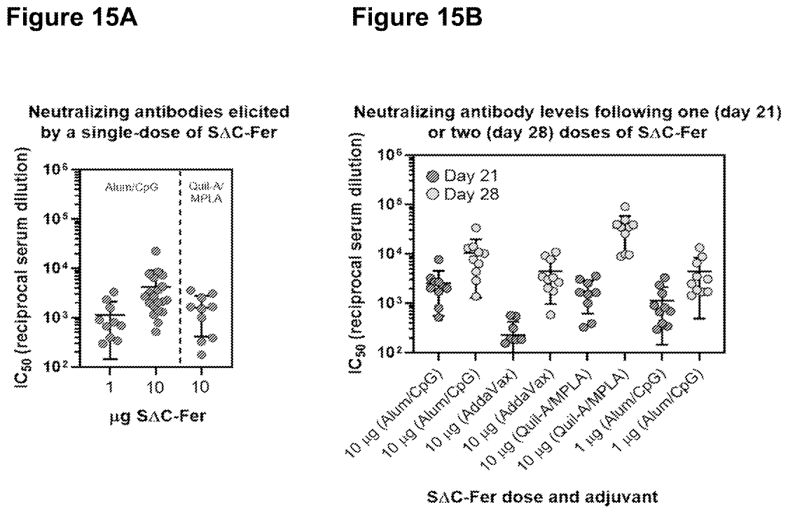

Screening of adjuvants and dosing conditions for immunization with SpikeΔC ferritin was conducted, with the results illustrated in FIGS. 15A and 15B. The neutralization properties of the sera collected from the experimental animals were assessed using a luciferase-based SARS-CoV-2 Spike pseudotyped lentiviral assay. FIG. 15A illustrates the comparison of adjuvant and dosing conditions for single-dose immunization with SpikeΔC ferritin. Experimental mice were subcutaneously administered a single dose of 1 μg or 10 μg of SpikeΔC ferritin adjuvanted with either 500 μg Alhydrogel® and 20 μg CpG, or 10 μg Quil-A® and 10 μg MPLA. The sera were collected at week 3 post-immunization. FIG. 15B illustrates the comparison of adjuvant and dosing conditions for one- and two-dose immunization with SpikeΔC ferritin. Experimental mice were subcutaneously administered a first (initial or prime) dose of 1 μg or 10 μg of SpikeΔC ferritin adjuvanted with either 500 μg Alhydrogel® and 20 μg CpG, AddaVax™, or 10 μg Quil-A® and 10 μg MPLA. The sera was collected at day 21 after the initial imisation, at which point the experimental mice were subcutaneously administered a second (boost) dose of 1 μg or 10 μg of SpikeΔC ferritin adjuvanted with either 500 μg Alhydrogel® and 20 μg CpG, AddaVax™, or 10 μg Quil-A® and 10 μg MPLA. The prime and the boost doses were identical in each group of experimental animals. The sera was also collected at day 28 after the initial immunization. The results illustrated in FIG. 15B showed that all the adjuvant conditions tested elicited quantifiable neutralizing antibody levels following immunization with SpikeΔC ferritin, with 500 μg Alhydrogel® and 20 μg CpG eliciting the most robust response following one dose, and 10 μg Quil-A® and 10 μg MPLA eliciting the most robust response following two doses.

### Example 9: Comparison of Neutralizing Antibody Responses Elicited by Two Different SARS-CoV-2 Spike Protein Antigens

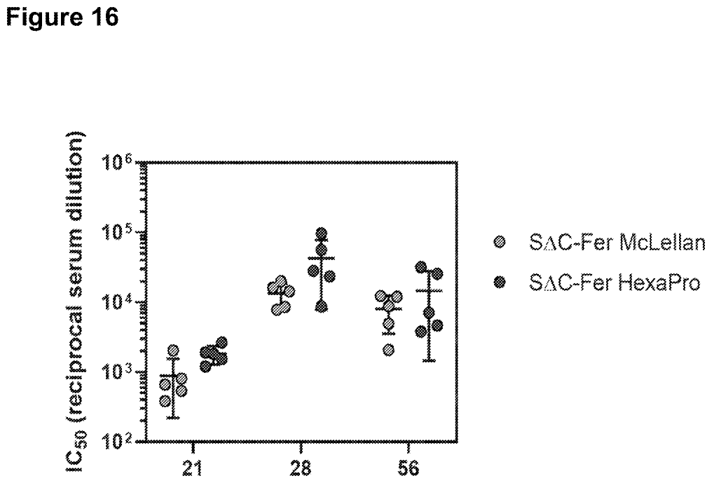

Comparison of neutralizing antibody responses elicited by two different SARS-CoV-2 Spike protein antigens, SpikeΔC ferritin (“SAC-Fer McLellan”) and SpikeHexaProAC ferritin (“SAC-Fer HexaPro”) was conducted, with the results illustrated in FIG. 16. SpikeHexaProAC ferritin (SEQ ID NO:16) was expressed and purified using the procedures substantially similar to those described in Example 1 and Hsieh et al., 2020. Using the procedures substantially similar to those described in Example 1, experimental mice were immunized with two doses 10 μg of SpikeΔC ferritin or SpikeHexaProAC ferritin adjuvanted with 10 μg Quil-A® and 10 μg MPL. The second (boost) dose was administered at day 21 after the initial immunization. The sera were collected at days 21, 28, and 56 after the initial immunization. The neutralization properties of the sera collected from the experimental mice were assessed using a luciferase-based SARS-CoV-2 Spike pseudotyped lentiviral assay. The comparison of neutralizing antibody responses elicited by SpikeΔC ferritin and SpikeHexaProAC ferritin revealed that SpikeHexaProAC ferritin was more immunogenic than SpikeΔC ferritin. SARS-CoV-2 Spike protein antigens based on HexaPro SARS-CoV-2 Spike protein sequence (SEQ ID NO:14) are shown below. SARS-CoV-2 Spike signal peptide sequences are shown in bold/underlined font, Hexahistidine tag sequences are shown in bold, Ser/Gly linker regions are underlined, GCN4 trimerization domain sequences are italicized, and H. pylori ferritin sequences are italicized and underlined.

SpikeHexaProΔC ferritin (“HexaPro ΔC ferritin”)-
SEQ ID NO: 16
MFVFLVLLPLVSSQCVNLTTRTQLPPAYTNSFTRGVYYPDKVFRSSVLHSTQDLFLPFFSNV
TWFHAIHVSGTNGTKRFDNPVLPFNDGVYFASTEKSNIIRGWIFGTTLDSKTQSLLIVNNAT
NVVIKVCEFQFCNDPFLGVYYHKNNKSWMESEFRVYSSANNCTFEYVSQPFLMDLEGKQGNF
KNLREFVFKNIDGYFKIYSKHTPINLVRDLPQGFSALEPLVDLPIGINITRFQTLLALHRSY
LTPGDSSSGWTAGAAAYYVGYLQPRTELLKYNENGTITDAVDCALDPLSETKCTLKSFTVEK
GIYQTSNERVQPTESIVRFPNITNLCPFGEVENATRFASVYAWNRKRISNCVADYSVLYNSA
SFSTFKCYGVSPTKLNDLCFTNVYADSFVIRGDEVRQIAPGQTGKIADYNYKLPDDFTGCVI
AWNSNNLDSKVGGNYNYLYRLFRKSNLKPFERDISTEIYQAGSTPCNGVEGENCYFPLQSYG
FQPTNGVGYQPYRVVVLSFELLHAPATVCGPKKSTNLVKNKCVNFNFNGLTGTGVLTESNKK
FLPFQQFGRDIADTTDAVRDPQTLEILDITPCSFGGVSVITPGTNTSNQVAVLYQDVNCTEV
PVAIHADQLTPTWRVYSTGSNVFQTRAGCLIGAEHVNNSYECDIPIGAGICASYQTQTNSPG
SASSVASQSIIAYTMSLGAENSVAYSNNSIAIPTNFTISVTTEILPVSMTKTSVDCTMYICG
DSTECSNLLLQYGSFCTQLNRALTGIAVEQDKNTQEVFAQVKQIYKTPPIKDFGGENFSQIL
PDPSKPSKRSPIEDLLENKVTLADAGFIKQYGDCLGDIAARDLICAQKENGLTVLPPLLTDE
MIAQYTSALLAGTITSGWTFGAGPALQIPFPMQMAYRENGIGVTQNVLYENQKLIANQFNSA
IGKIQDSLSSTPSALGKLQDVVNQNAQALNTLVKQLSSNFGAISSVLNDILSRLDPPEAEVQ
IDRLITGRLQSLQTYVTQQLIRAAEIRASANLAATKMSECVLGQSKRVDFCGKGYHLMSFPQ
SAPHGVVFLHVTYVPAQEKNFTTAPAICHDGKAHFPREGVFVSNGTHWFVTQRNFYEPQIIT
TDNTFVSGNCDVVIGIVNNTVYDPLQPELDSGGDIIKLLNEQVNKEMQSSNLYMSMSSWCYT
HSLDGAGLFLFDHAAEEYEHAKKLIIFLNENNVPVQLTSISAPEHKFEGLTQIFQKAYEHEQ
HISESINNIVDHAIKSKDHATFNFLQWYVAEQHEEEVLENDILDKIELIGNENHGLYLADQY
VKGIAKSRKS
Spike HexaProΔC ferritin variant (“HexaPro ΔC ferritin variant”)-
SEQ ID NO: 17
MFVFLVLLPLVSSQCVNLTTRTQLPPAYTNSFTRGVYYPDKVFRSSVLHSTQDLFLPFFSNV
TWFHAIHVSGTNGTKRFDNPVLPFNDGVYFASTEKSNIIRGWIFGTTLDSKTQSLLIVNNAT
NVVIKVCEFQFCNDPFLGVYYHKNNKSWMESEFRVYSSANNCTFEYVSQPFLMDLEGKQGNF
KNLREFVEKNIDGYFKIYSKHTPINLVRDLPQGFSALEPLVDLPIGINITRFQTLLALHRSY
LTPGDSSSGWTAGAAAYYVGYLQPRTELLKYNENGTITDAVDCALDPLSETKCTLKSFTVEK
GIYQTSNERVQPTESIVRFPNITNLCPFGEVENATRFASVYAWNRKRISNCVADYSVLYNSA
SFSTFKCYGVSPTKLNDLCFTNVYADSFVIRGDEVRQIAPGQTGKIADYNYKLPDDFTGCVI
AWNSNNLDSKVGGNYNYLYRLFRKSNLKPFERDISTEIYQAGSTPCNGVEGENCYFPLQSYG
FQPTNGVGYQPYRVVVLSFELLHAPATVCGPKKSTNLVKNKCVNFNFNGLTGTGVLTESNKK
FLPFQQFGRDIADTTDAVRDPQTLEILDITPCSFGGVSVITPGTNTSNQVAVLYQDVNCTEV
PVAIHADQLTPTWRVYSTGSNVFQTRAGCLIGAEHVNNSYECDIPIGAGICASYQTQTNSPG
SASSVASQSIIAYTMSLGAENSVAYSNNSIAIPTNFTISVTTEILPVSMTKTSVDCTMYICG
DSTECSNLLLQYGSFCTQLNRALTGIAVEQDKNTQEVFAQVKQIYKTPPIKDFGGENFSQIL
PDPSKPSKRSPIEDLLFNKVTLADAGFIKQYGDCLGDIAARDLICAQKENGLTVLPPLLTDE
MIAQYTSALLAGTITSGWTFGAGPALQIPFPMQMAYRENGIGVTQNVLYENQKLIANQENSA
IGKIQDSLSSTPSALGKLQDVVNQNAQALNTLVKQLSSNFGAISSVLNDILSRLDPPEAEVQ
IDRLITGRLQSLQTYVTQQLIRAAEIRASANLAATKMSECVLGQSKRVDFCGKGYHLMSFPQ
SAPHGVVFLHVTYVPAQEKNFTTAPAICHDGKAHFPREGVFVSNGTHWFVTQRNFYEPQIIT
TDNTFVSGNCDVVIGIVNNTVYDPLQPELDSGGDIIKLLNEQVNKEMQSSNLYMSMSSWCYT
HSLDGAGLFLFDHAAEEYEHAKKLIIFLNENNVPVQLTSISAPEHKFEGLTQIFQKAYEHEQ
HISESINNIVDHAIKSKDHATFNFLQWYVAEQHEEEVLEKDILDKIELIGNENHGLYLADQY
VKGIAKSRKS
Spike HexaPro ferritin (“HexaPro ferritin”)-
SEQ ID NO: 18
MFVFLVLLPLVSSQCVNLTTRTQLPPAYTNSFTRGVYYPDKVFRSSVLHSTQDLFLPFFSNV
TWFHAIHVSGTNGTKRFDNPVLPFNDGVYFASTEKSNIIRGWIFGTTLDSKTQSLLIVNNAT
NVVIKVCEFQFCNDPFLGVYYHKNNKSWMESEFRVYSSANNCTFEYVSQPFLMDLEGKQGNF
KNLREFVFKNIDGYFKIYSKHTPINLVRDLPQGFSALEPLVDLPIGINITRFQTLLALHRSY
LTPGDSSSGWTAGAAAYYVGYLQPRTFLLKYNENGTITDAVDCALDPLSETKCTLKSFTVEK
GIYQTSNFRVQPTESIVRFPNITNLCPFGEVENATRFASVYAWNRKRISNCVADYSVLYNSA
SFSTFKCYGVSPTKLNDLCFTNVYADSFVIRGDEVRQIAPGQTGKIADYNYKLPDDFTGCVI
AWNSNNLDSKVGGNYNYLYRLFRKSNLKPFERDISTEIYQAGSTPCNGVEGENCYFPLQSYG
FQPTNGVGYQPYRVVVLSFELLHAPATVCGPKKSTNLVKNKCVNFNFNGLTGTGVLTESNKK
FLPFQQFGRDIADTTDAVRDPQTLEILDITPCSFGGVSVITPGTNTSNQVAVLYQDVNCTEV
PVAIHADQLTPTWRVYSTGSNVFQTRAGCLIGAEHVNNSYECDIPIGAGICASYQTQTNSPG
SASSVASQSIIAYTMSLGAENSVAYSNNSIAIPTNFTISVTTEILPVSMTKTSVDCTMYICG
DSTECSNLLLQYGSFCTQLNRALTGIAVEQDKNTQEVFAQVKQIYKTPPIKDFGGENFSQIL
PDPSKPSKRSPIEDLLENKVTLADAGFIKQYGDCLGDIAARDLICAQKENGLTVLPPLLTDE
MIAQYTSALLAGTITSGWTFGAGPALQIPFPMQMAYRENGIGVTQNVLYENQKLIANQFNSA
IGKIQDSLSSTPSALGKLQDVVNQNAQALNTLVKQLSSNFGAISSVLNDILSRLDPPEAEVQ
IDRLITGRLQSLQTYVTQQLIRAAEIRASANLAATKMSECVLGQSKRVDFCGKGYHLMSFPQ
SAPHGVVFLHVTYVPAQEKNFTTAPAICHDGKAHFPREGVFVSNGTHWFVTQRNFYEPQIIT
TDNTFVSGNCDVVIGIVNNTVYDPLQPELDSFKEELDKYFKNHTSPDVDLGDISGINASVVN
IQKEIDRLNEVAKNLNESLIDLQELGKYEQSGGDIIKLLNEQVNKEMQSSNLYMSMSSWCYT
HSLDGAGLFLFDHAAEEYEHAKKLIIFLNENNVPVQLTSISAPEHKFEGLTQIFQKAYEHEQ
HISESINNIVDHAIKSKDHATFNFLQWYVAEQHEEEVLEKDILDKIELIGNENHGLYLADQY
VKGIAKSRKS
SpikeHexaPro GCN4 (“HexaPro GCN4”)-
SEQ ID NO: 19
MFVFLVLLPLVSSQCVNLTTRTQLPPAYTNSFTRGVYYPDKVFRSSVLHSTQDLFLPFFSNV
TWFHAIHVSGTNGTKRFDNPVLPENDGVYFASTEKSNIIRGWIFGTTLDSKTQSLLIVNNAT
NVVIKVCEFQFCNDPFLGVYYHKNNKSWMESEFRVYSSANNCTFEYVSQPFLMDLEGKQGNE
KNLREFVFKNIDGYFKIYSKHTPINLVRDLPQGFSALEPLVDLPIGINITRFQTLLALHRSY
LTPGDSSSGWTAGAAAYYVGYLQPRTFLLKYNENGTITDAVDCALDPLSETKCTLKSFTVEK
GIYQTSNERVQPTESIVRFPNITNLCPFGEVENATRFASVYAWNRKRISNCVADYSVLYNSA
SFSTFKCYGVSPTKLNDLCFTNVYADSFVIRGDEVRQIAPGQTGKIADYNYKLPDDETGCVI
AWNSNNLDSKVGGNYNYLYRLFRKSNLKPFERDISTEIYQAGSTPCNGVEGENCYFPLQSYG
FQPTNGVGYQPYRVVVLSFELLHAPATVCGPKKSTNLVKNKCVNFNFNGLTGTGVLTESNKK
FLPFQQFGRDIADTTDAVRDPQTLEILDITPCSFGGVSVITPGTNTSNQVAVLYQDVNCTEV
PVAIHADQLTPTWRVYSTGSNVFQTRAGCLIGAEHVNNSYECDIPIGAGICASYQTQTNSPG
SASSVASQSIIAYTMSLGAENSVAYSNNSIAIPTNFTISVTTEILPVSMTKTSVDCTMYICG
DSTECSNLLLQYGSFCTQLNRALTGIAVEQDKNTQEVFAQVKQIYKTPPIKDFGGENFSQIL
PDPSKPSKRSPIEDLLENKVTLADAGFIKQYGDCLGDIAARDLICAQKENGLTVLPPLLTDE
MIAQYTSALLAGTITSGWTFGAGPALQIPFPMQMAYRENGIGVTQNVLYENQKLIANQENSA
IGKIQDSLSSTPSALGKLQDVVNQNAQALNTLVKQLSSNFGAISSVLNDILSRLDPPEAEVQ
IDRLITGRLQSLQTYVTQQLIRAAEIRASANLAATKMSECVLGQSKRVDFCGKGYHLMSFPQ
SAPHGVVFLHVTYVPAQEKNFTTAPAICHDGKAHFPREGVFVSNGTHWFVTQRNFYEPQIIT
TDNTFVSGNCDVVIGIVNNTVYDPLQPELDSFKEELDKYFKNHTSPDVDLGDISGINASVVN
IQKEIDRLNEVAKNLNESLIDLQELGKYEQGGGGSRMKQIEDKIEEILSKQYHIENEIARIK
KLIGERGGSGGHHHHHH
SpikeHexaProΔC GCN4 (“HexaPro ΔC GCN4”)-
SEQ ID NO: 20
MFVFLVLLPLVSSQCVNLTTRTQLPPAYTNSFTRGVYYPDKVFRSSVLHSTQDLFLPFFSNV
TWFHAIHVSGTNGTKRFDNPVLPFNDGVYFASTEKSNIIRGWIFGTTLDSKTQSLLIVNNAT
NVVIKVCEFQFCNDPFLGVYYHKNNKSWMESEFRVYSSANNCTFEYVSQPFLMDLEGKQGNE
KNLREFVFKNIDGYFKIYSKHTPINLVRDLPQGFSALEPLVDLPIGINITRFQTLLALHRSY
LTPGDSSSGWTAGAAAYYVGYLQPRTFLLKYNENGTITDAVDCALDPLSETKCTLKSFTVEK
GIYQTSNFRVQPTESIVRFPNITNLCPFGEVENATRFASVYAWNRKRISNCVADYSVLYNSA
SFSTFKCYGVSPTKLNDLCFTNVYADSFVIRGDEVRQIAPGQTGKIADYNYKLPDDFTGCVI
AWNSNNLDSKVGGNYNYLYRLFRKSNLKPFERDISTEIYQAGSTPCNGVEGENCYFPLQSYG
FQPTNGVGYQPYRVVVLSFELLHAPATVCGPKKSTNLVKNKCVNFNFNGLTGTGVLTESNKK
FLPFQQFGRDIADTTDAVRDPQTLEILDITPCSFGGVSVITPGTNTSNQVAVLYQDVNCTEV
PVAIHADQLTPTWRVYSTGSNVFQTRAGCLIGAEHVNNSYECDIPIGAGICASYQTQTNSPG
SASSVASQSIIAYTMSLGAENSVAYSNNSIAIPTNFTISVTTEILPVSMTKTSVDCTMYICG
DSTECSNLLLQYGSFCTQLNRALTGIAVEQDKNTQEVFAQVKQIYKTPPIKDFGGENFSQIL
PDPSKPSKRSPIEDLLENKVTLADAGFIKQYGDCLGDIAARDLICAQKENGLTVLPPLLTDE
MIAQYTSALLAGTITSGWTFGAGPALQIPFPMQMAYRENGIGVTQNVLYENQKLIANQFNSA
IGKIQDSLSSTPSALGKLQDVVNQNAQALNTLVKQLSSNFGAISSVLNDILSRLDPPEAEVQ
IDRLITGRLQSLQTYVTQQLIRAAEIRASANLAATKMSECVLGQSKRVDFCGKGYHLMSFPQ
SAPHGVVFLHVTYVPAQEKNFTTAPAICHDGKAHFPREGVFVSNGTHWFVTQRNFYEPQIIT
TDNTFVSGNCDVVIGIVNNTVYDPLQPELDGGGGSRMKQIEDKIEEILSKQYHIENEIARIK
KLIGERGGSGGHHHHHH

### Example 10: Comparison of Expression and Purification Yields of Three Different SARS-CoV-2 Spike Protein Antigens

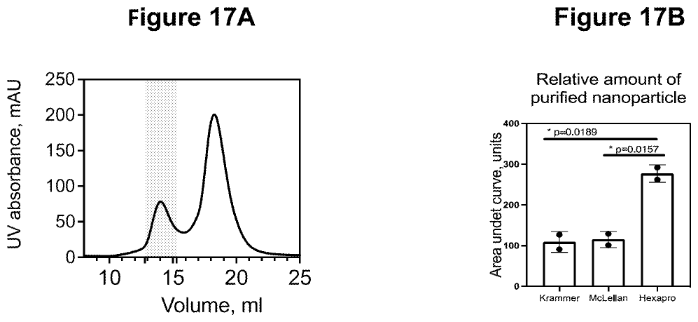

Expression and purification yields of the following SARS-CoV-2 Spike protein antigens were compared: AC Spike ferritin fusion protein (“SpikeΔC ferritin,” SEQ ID NO:13, denoted as “Krammer” in FIGS. 17B-19), AC Spike ferritin fusion protein variant (“SpikeΔC ferritin variant,” SEQ ID NO:21, denoted as “McLellan” in FIGS. 17B-19), and SpikeHexaProAC ferritin (“HexaPro AC ferritin,” SEQ ID NO:16, denoted as “HexaPro” in FIGS. 17B-19) was conducted, with the results illustrated in FIGS. 17A and 17B. Amino acid sequence of AC Spike ferritin fusion protein variant (SEQ ID NO:14) is shown below. SARS-CoV-2 Spike signal peptide sequence is shown in bold/underlined font, Ser/Gly linker region is underlined, and H. pylori ferritin sequences are italicized and underlined.

ΔC Spike ferritin fusion protein variant
(“SpikeΔC ferritin variant”)-
SEQ ID NO: 21
MFVFLVLLPLVSSQCVNLTTRTQLPPAYTNSFTRGVYYPDKVFRSSVLHSTQDLFLPFFSNV
TWFHAIHVSGTNGTKRFDNPVLPENDGVYFASTEKSNIIRGWIFGTTLDSKTQSLLIVNNAT
NVVIKVCEFQFCNDPFLGVYYHKNNKSWMESEFRVYSSANNCTFEYVSQPFLMDLEGKQGNE
KNLREFVFKNIDGYFKIYSKHTPINLVRDLPQGFSALEPLVDLPIGINITRFQTLLALHRSY
LTPGDSSSGWTAGAAAYYVGYLQPRTFLLKYNENGTITDAVDCALDPLSETKCTLKSFTVEK
GIYQTSNFRVQPTESIVRFPNITNLCPFGEVENATRFASVYAWNRKRISNCVADYSVLYNSA
SFSTFKCYGVSPTKLNDLCFTNVYADSFVIRGDEVRQIAPGQTGKIADYNYKLPDDFTGCVI
AWNSNNLDSKVGGNYNYLYRLFRKSNLKPFERDISTEIYQAGSTPCNGVEGENCYFPLQSYG
FQPTNGVGYQPYRVVVLSFELLHAPATVCGPKKSTNLVKNKCVNFNFNGLTGTGVLTESNKK
FLPFQQFGRDIADTTDAVRDPQTLEILDITPCSFGGVSVITPGTNTSNQVAVLYQDVNCTEV
PVAIHADQLTPTWRVYSTGSNVFQTRAGCLIGAEHVNNSYECDIPIGAGICASYQTQTNSPG
SASSVASQSIIAYTMSLGAENSVAYSNNSIAIPTNFTISVTTEILPVSMTKTSVDCTMYICG
DSTECSNLLLQYGSFCTQLNRALTGIAVEQDKNTQEVFAQVKQIYKTPPIKDFGGFNFSQIL
PDPSKPSKRSFIEDLLENKVTLADAGFIKQYGDCLGDIAARDLICAQKENGLTVLPPLLTDE
MIAQYTSALLAGTITSGWTFGAGAALQIPFAMQMAYRENGIGVTQNVLYENQKLIANQFNSA
IGKIQDSLSSTASALGKLQDVVNQNAQALNTLVKQLSSNFGAISSVLNDILSRLDPPEAEVQ
IDRLITGRLQSLQTYVTQQLIRAAEIRASANLAATKMSECVLGQSKRVDFCGKGYHLMSFPQ
SAPHGVVFLHVTYVPAQEKNFTTAPAICHDGKAHFPREGVFVSNGTHWFVTQRNFYEPQIIT
TDNTFVSGNCDVVIGIVNNTVYDPLQPELDSGGDIIKLLNEQVNKEMQSSNLYMSMSSWCYT
HSLDGAGLFLFDHAAEEYEHAKKLIIFLNENNVPVQLTSISAPEHKFEGLTQIFQKAYEHEQ
HISESINNIVDHAIKSKDHATFNFLQWYVAEQHEEEVLFKDILDKIELIGNENHGLYLADQY
VKGIAKSRKS

Each of the above three SARS-CoV-2 Spike protein antigens was expressed and purified using the procedures based on those described in Example 1 and Hsieh et al., 2020. The expression, which was performed in duplicate for each SARS-CoV-2 Spike protein, was conducted in Expi293F cells cultured in medium containing Freestyle™ and Expi293™ expression media (Thermo Fisher Scientific, Waltham, Massachusetts) mixed at 2:1 ratio and transfected with FectPRO® reagent (Polyplus transfection, New York, New York) according to the manufacturer's instructions. After 4-5 days of culture, the culture medium was clarified by spinning and filtration. Clarified media was diluted with 20 mM Tris, pH 8.0, buffer and loaded with a sample pump on HiTrap Q® HP (Cytiva, Marlborough, Massachusetts) column pre-equilibrated with a low ionic strength buffer (Buffer A, 10 mM Tris, pH 8.0). The column was washed with 5 column volumes of Buffer A and a gradient of Buffer B (10 mM Tris, pH 8.0, 1M NaCl) was applied. The fractions eluted with 5-25% buffer B were collected and concentrated 20-fold using centrifugal concentrators (Amicon®, MilliporeSigma, Burlington, Massachusetts), 100 kDa cutoff). The resulting concentrate was diluted 10 times by PBS and concentrated again with the centrifugal concentrators. AKTA™ pure FPLC (Cytiva, Marlborough, Massachusetts) system with SRT1000 gel filtration column was used for further purification.

For gel filtration, 2 ml of sample was injected into the FPLC system using a 2 ml loop and applied to a SRT1000 column pre-equilibrated with degassed PBS buffer. The fractions containing SARS-CoV-2 Spike protein antigen were collected, pooled and concentrated with the centrifugal concentrators. Glycerol or sucrose was added to the concentrated samples to final concentration of 10% (by weight for sucrose or by volume for glycerol) which were then filtered with 0.22 μm filters and flash-frozen with liquid nitrogen at 0.4-0.5 mg/ml. FIG. 17A shows a representative size-exclusion chromatography trace of a SARS-CoV-2 Spike protein antigen, with the pooled fractions shaded. A relative amount of each a SARS-CoV-2 Spike protein obtained was calculated as a shaded area under the curve representing the fractions containing SARS-CoV-2 Spike protein antigen (illustrated in FIG. 17A). FIG. 17B illustrates a comparison of relative amounts of each SARS-CoV-2 Spike protein antigen obtained by the above-described expression and purification procedure. The comparison illustrated in FIG. 17B revealed that the yield of SpikeHexaProAC ferritin was approximately 2.5 higher than the yield of either SpikeΔC ferritin, or SpikeΔC ferritin variant.

### Example 11: Immunogenicity of Three Different SARS-CoV-2 Spike Protein Antigens

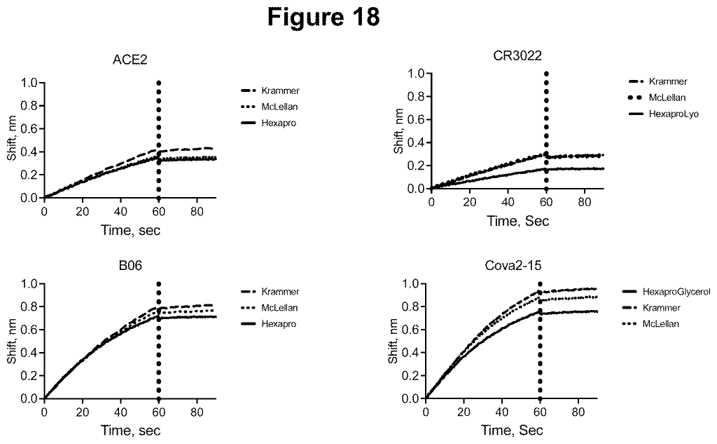

Potential immunogenicity of each of the three SARS-CoV-2 Spike protein antigens described in Example 10 was assessed. Bio-layer interferometry (BLI) on the Octet® system (Sartorius, Gottingen, Germany) was used to test binding of SARS-CoV-2 Spike protein antigens to the conformational monoclonal antibodies (mAbs) and to ACE2 receptor. Variable heavy chain (HC) and variable light chain (LC) sequences for SARS-CoV-2 reactive mAbs, CR3022 (HC GenBank DQ168569, LC Genbank DQ168570), CB6 (HC GenBank MT470197, LC GenBank MT470196), and COVA-2-15 (HC GenBank MT599861, LC GenBank MT599945) were codon-optimized for human expression using the IDT Codon Optimization Tool and ordered as gene-block fragments from IDT. The fragments were amplified by PCR and inserted, using In-Fusion® cloning system (Takara Bio, Shiga, Japan), into CMV/R expression vectors containing heavy chain or light chain Fc sequence from VRC01. Soluble human ACE2 with an Fc tag was constructed by PCR-amplifying ACE2 (sequence encoding amino acid residues 1-615) from Addgene plasmid #1786 and fusing it to a human Fc domain from VRC01, separated by a TEV-GSGG (SEQ ID NO:5) linker using a stitching PCR step. ACE2-Fc was inserted into the pADD2 mammalian expression vector via In-Fusion® using EcoRI/XhoI cut sites. SARS-CoV-2 mAbs to purified spike nanoparticles and ACE2 receptor-Fc fusion protein were loaded on Octet Fc-binding tips at 100 nM concentration, and the tips were dipped into wells with SARS-CoV-2 Spike protein antigen being tested diluted to 150 nM (SARS-CoV-2 Spike protein antigen monomer concentration) with Octet binding buffer. After 60 seconds of association, the tips were moved into wells with only buffer present (in order to measure dissociation). Equivalent binding of each of the three SARS-CoV-2 Spike protein antigens to conformational antibodies and ACE2 receptor was observed, as illustrated by FIG. 18. The above experimental observations confirmed that each of the three SARS-CoV-2 Spike protein antigens displayed epitopes in a similar manner and demonstrated that the presentation of the immunogenic sites was not affected by the sequence differences among the tested SARS-CoV-2 Spike protein antigens.

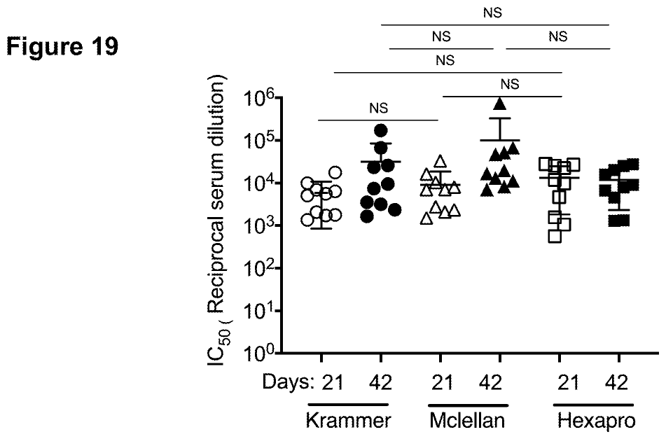

Comparison of neutralizing antibody responses elicited by SARS-CoV-2 Spike protein antigens was conducted using the following immunization scheme. Ten mice per group were immunized with two doses of 10 μg of each SARS-CoV-2 Spike protein antigen adjuvanted with 500 μg Alum (InvinoGen, San Diego, California) and 20 μg CpG (InvivoGen). The doses were administered by intramuscular injection on “Day 0” and “Day 21,” and blood samples were drawn on “Day 0” (prior to immunization), “Day 21,” and “Day 42.” The neutralization titers of the sera collected from the experimental animals were assessed using SARS-CoV-2 Spike pseudotyped lentivirus neutralization assay described in Example 1. The infectivity of the cells were measured after 48 hours by measuring luciferase enzyme activity. The relative luciferase enzyme activity was plotted against the serum dilution and the 50% infective concentration (IC50) was calculated from the dilution curves. The results are illustrated in FIG. 19. The three SARS-CoV-2 Spike protein antigens tested produced neutralization titers that were not statistically different.

### Example 12: Lyophilization of SARS-CoV-2 Spike Protein Antigen

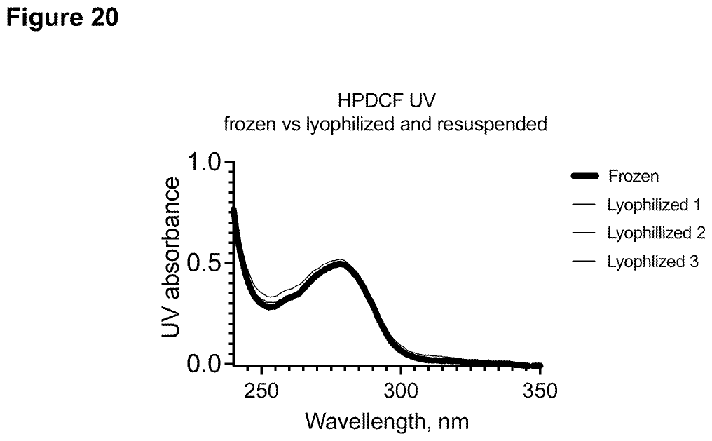

Experimental studies of lyophilized SpikeHexaProAC ferritin were conducted and demonstrated that SpikeHexaProAC ferritin lyophilized in presence of sucrose and subsequently reconstituted retained its structure and immunogenicity. The results of the experimental studies are illustrated in FIGS. 20-26. For the first series of studies, SpikeHexaProAC ferritin was expressed and purified as described in Example 10 and flash frozen in PBS with 10% sucrose. To generate lyophilized and reconstituted SpikeHexaProAC ferritin (“lyophilized samples”), frozen samples were lyophilized overnight on a freeze dryer (Labconco™, Kansas City, Missouri) and resuspended in a volume of water equal to the starting volume of PBS with 10% sucrose.

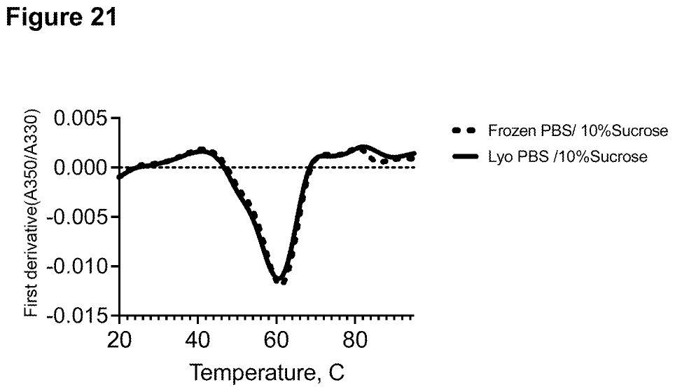

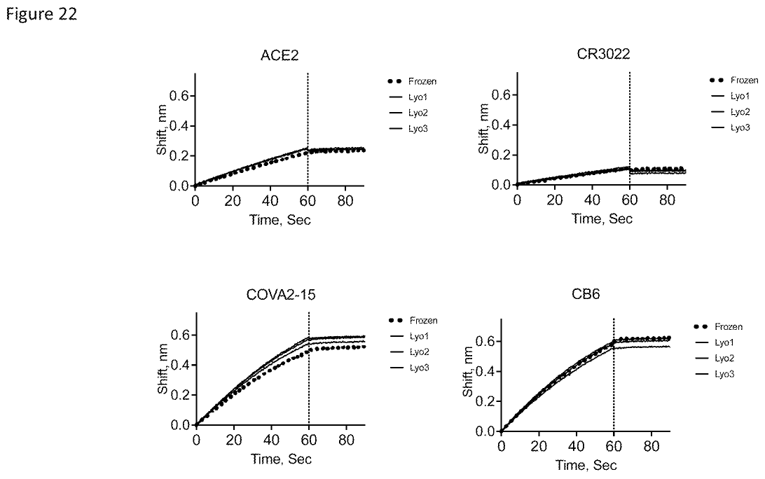

To confirm the that SpikeHexaProAC ferritin can be lyophilized and reconstituted without loss, the UV absorbance spectra of frozen and thawed SpikeHexaProAC ferritin samples (“frozen samples”) and of the lyophilized samples were compared, with the results illustrated in FIG. 20. Differential scanning fluorimetry (the results are illustrated in FIG. 21) confirmed that SpikeHexaProAC ferritin had the same thermal stability in frozen and lyophilized samples. To confirm that SpikeHexaProAC ferritin in the lyophilized samples retained its conformational epitopes, both samples were tested by BLI substantially as described in Example 11. The results of BLI analysis are illustrated in FIG. 22. BLI analysis showed that frozen and lyophilized samples bound to conformational antibodies and to the ACE receptor in a similar manner, demonstrating that the presentation of the immunogenic sites was not affected by lyophilization and reconstitution.

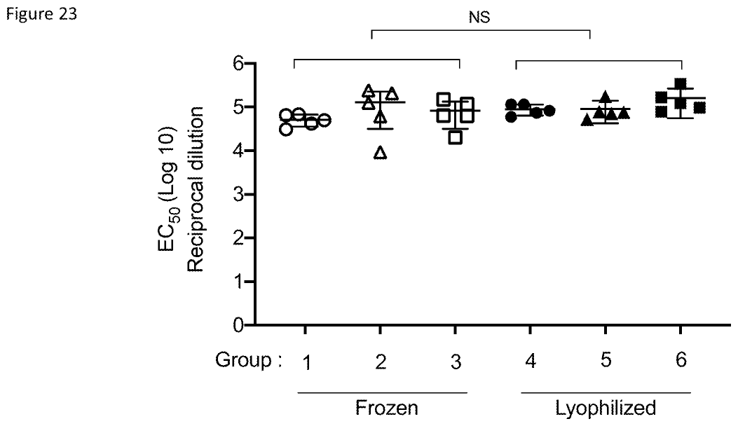

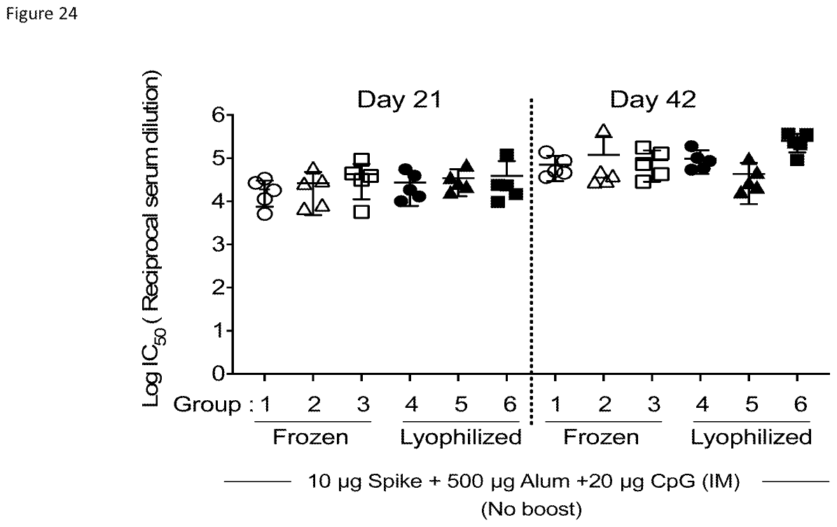

The immunogenicity of lyophilized and reconstituted SpikeHexaProAC ferritin was compared to the immunogenicity of frozen and thawed SpikeHexaProAC ferritin. Frozen and lyophilized samples were administered to three identical groups of five mice each (six groups total). Prior to administration, lyophilized and frozen samples were incubated at room temperature for 1 hour. After 1 hour, the samples were formulated by mixing 10 μg of protein with 500 μg Alum and 20 μg CpG. The mice were primed by immunization via intra-muscular injection on “Day 0,” and blood samples were collected on “Day 0” before priming, “Day 21,” and “Day 42” after immunization. The binding of the antisera to SARS-CoV-2 RBD protein was measured on “Day 21.” 96-well plates were coated with recombinant SARS-CoV-2 RBD protein, and the titers of diluted serum samples were measured by ELISA. Optical densities were plotted against serum dilution, and 50% effective concentrations (EC50) were calculated from the dilution curves. The results are illustrated in FIG. 23. SARS-CoV-2 pseudovirus neutralization titers were tested on “Day 21” and “Day 42.” Diluted mouse serum samples were incubated with pseudo-typed SARS-CoV-2 virus harboring “Delta 21-Spike” protein (SARS-CoV-2 Spike protein with C-terminal 21 amino acids deletion) and luciferase for 1 hour, and the added onto HeLa cells expressing ACE2 and transmembrane serine protease 2 (TMPRSS2). The infectivity of the cells was measured after 48 hours by measuring luciferase enzyme activity. The relative luciferase enzyme activity was plotted against the serum dilution and the 50% infective concentration (IC50) was calculated from the dilution curves. The results are illustrated in FIG. 24. The above studies showed that RBD binding titers and SARS-CoV-2 pseudovirus neutralization titers were not statistically different between the sera from mice immunized with frozen and lyophilized vaccine candidates.

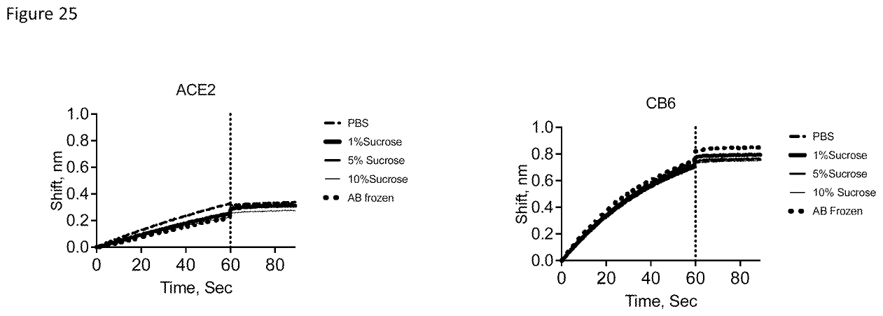

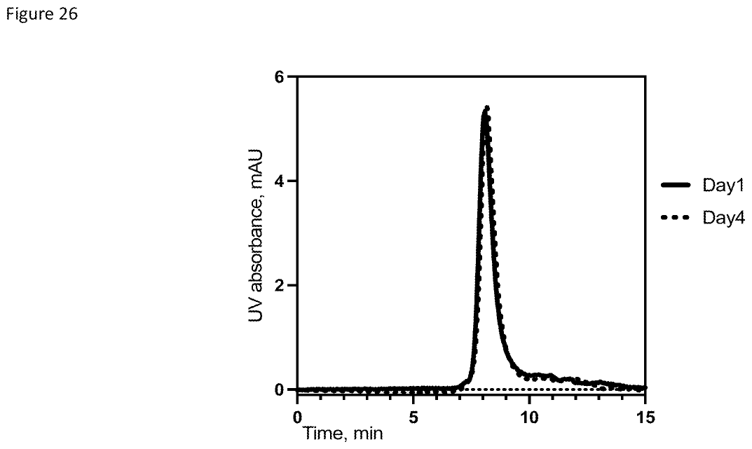

It was demonstrated that SpikeHexaProAC ferritin can be lyophilized in volatile ammonium bicarbonate buffer and resuspended at concentrations above 10 mg/ml. Lyophilization in non-volatile buffers, such as PBS, necessitates resuspension in comparable volumes of water to prevent a buildup of very high salt concentrations post-reconstitution. Using a volatile buffer allows for the protein to be resuspended in smaller volume compared to the starting volume, increasing the sample concentration. For the lyophilization in ammonium bicarbonate buffer, 1% sucrose (by weight) was used as a stabilizing agent. 1% sucrose was chosen based of ease of reconstitution (solubilization) of the lyophilized sample. SpikeHexaProAC ferritin was expressed and purified as described in Example 10, dialyzed overnight into 10 mM ammonium bicarbonate, pH 7.8. After dialysis, sucrose was added to 1% final concentration (by weight). The sample was then flash frozen at 1 mg/ml protein concentration in liquid nitrogen, lyophilized overnight, and resuspended in PBS at protein concentration of approximately 11 mg/ml. The reconstituted samples was then tested for binding to the conformational antibody CB6 and ACE2 receptor by BLI (the results are illustrated in FIG. 25). Structural integrity of the SpikeHexaProAC ferritin nanoparticles in the sample was confirmed by size exclusion chromatography—multiple angle light scattering (SEC-MALS). The results of SEC-MALS experiments are illustrated in FIG. 26. FIG. 26 illustrates the results of SEC-MALS testing the properties of SARS-CoV-2 Spike protein antigen lyophilized in volatile ammonium bicarbonate buffer. For the SEC-MALS experiment, 5 μg of protein was loaded, directly after reconstitution, onto SRT SEC-1000 4.6×300 mm column equilibrated in PBS. A single prominent peak detected in in both the UV and light-scattering traces confirmed that the nanoparticles in the sample were homogeneous and did not aggregate. The sample was then stored at room temperature for 4 days, and the SEC-MALS experiment was repeated to verify sample

### Example 13: Decreasing Ferritin Domain Immunogenicity by Engineered Glycosylation

In order to decrease immunogenicity of the ferritin domain of SARS-CoV-2 Spike ferritin fusion protein antigens according to certain embodiments of the present disclosure, artificial glycosylation sites were designed to be installed into the ferritin domain. The ferritin domain of the fusion proteins according to the present disclosure do not contain the naturally occurring consensus sequence N-X-S/T (where X cannot be P) that is required for N-linked protein glycosylation. To construct an artifical glycosysiation site in the ferritin domain, a position was selected that was distant from the 3-fold axis of symmetry of a fusion protein nanoparticle, and two amino acid substitutions were introduced, resulting in an arficial glycosylation site. Selecting a position that is far from the 3-fold axis of symmetry is envisioned to minimize disruptions of the immune response to the Spike protein domain (which is located at the 3 fold axis) of SARS-CoV-2 Spike fusion protein antigen. Examples of SpikeHexaProAC ferritin variants with artificial glycosylation sites are shown as SEQ ID NOs 22-25. SARS-CoV-2 Spike signal peptide sequences are shown in bold/underlined font, Ser/Gly linker regions are underlined, and H. pylori ferritin sequences are italicized and underlined, and amino acid substitutions in the ferritin domain are italicized, underlined and bolded.

site variant 1 (“HexaPro ΔC Gly 1 ferritin”)-
SEQ ID NO: 22
MFVFLVLLPLVSSQCVNLTTRTQLPPAYTNSFTRGVYYPDKVERSSVLHSTQDLFLPFFSNV
TWFHAIHVSGTNGTKRFDNPVLPENDGVYFASTEKSNIIRGWIFGTTLDSKTQSLLIVNNAT
NVVIKVCEFQFCNDPFLGVYYHKNNKSWMESEFRVYSSANNCTFEYVSQPFLMDLEGKQGNF
KNLREFVFKNIDGYFKIYSKHTPINLVRDLPQGFSALEPLVDLPIGINITRFQTLLALHRSY
LTPGDSSSGWTAGAAAYYVGYLQPRTFLLKYNENGTITDAVDCALDPLSETKCTLKSFTVEK
GIYQTSNERVQPTESIVRFPNITNLCPFGEVENATRFASVYAWNRKRISNCVADYSVLYNSA
SFSTFKCYGVSPTKLNDLCFTNVYADSFVIRGDEVRQIAPGQTGKIADYNYKLPDDFTGCVI
AWNSNNLDSKVGGNYNYLYRLFRKSNLKPFERDISTEIYQAGSTPCNGVEGENCYFPLQSYG
FQPTNGVGYQPYRVVVLSFELLHAPATVCGPKKSTNLVKNKCVNFNFNGLTGTGVLTESNKK
FLPFQQFGRDIADTTDAVRDPQTLEILDITPCSFGGVSVITPGTNTSNQVAVLYQDVNCTEV
PVAIHADQLTPTWRVYSTGSNVFQTRAGCLIGAEHVNNSYECDIPIGAGICASYQTQTNSPG
SASSVASQSIIAYTMSLGAENSVAYSNNSIAIPTNFTISVTTEILPVSMTKTSVDCTMYICG
DSTECSNLLLQYGSFCTQLNRALTGIAVEQDKNTQEVFAQVKQIYKTPPIKDFGGFNFSQIL
PDPSKPSKRSPIEDLLENKVTLADAGFIKQYGDCLGDIAARDLICAQKENGLTVLPPLLTDE
MIAQYTSALLAGTITSGWTFGAGPALQIPFPMQMAYRENGIGVTQNVLYENQKLIANQENSA
IGKIQDSLSSTPSALGKLQDVVNQNAQALNTLVKQLSSNFGAISSVLNDILSRLDPPEAEVQ
IDRLITGRLQSLQTYVTQQLIRAAEIRASANLAATKMSECVLGQSKRVDFCGKGYHLMSFPQ
SAPHGVVFLHVTYVPAQEKNFTTAPAICHDGKAHFPREGVFVSNGTHWFVTQRNFYEPQIIT
TDNTFVSGNCDVVIGIVNNTVYDPLQPELDSGGDIIKLLNEQVNKEMQSSNLYMSMSSWCYT
HSLDGAGLFLFDHAAEEYEHAKKLIIFLNENNVPVQLTSISAPEHNFTGLTQIFQKAYEHEQ
HISESINNIVDHAIKSKDHATFNFLQWYVAEQHEEEVLFKDILDKIELIGNENHGLYLADQY
VKGIAKSRKS
SpikeHexaProΔC ferritin with artificial glycosylation
site variant 2 (“HexaPro ΔC Gly 2 ferritin”)-
SEQ ID NO: 23
MFVFLVLLPLVSSQCVNLTTRTQLPPAYTNSFTRGVYYPDKVFRSSVLHSTQDLFLPFFSNV
TWFHAIHVSGTNGTKRFDNPVLPENDGVYFASTEKSNIIRGWIFGTTLDSKTQSLLIVNNAT
NVVIKVCEFQFCNDPFLGVYYHKNNKSWMESEFRVYSSANNCTFEYVSQPFLMDLEGKQGNF
KNLREFVFKNIDGYFKIYSKHTPINLVRDLPQGFSALEPLVDLPIGINITRFQTLLALHRSY
LTPGDSSSGWTAGAAAYYVGYLQPRTFLLKYNENGTITDAVDCALDPLSETKCTLKSFTVEK
GIYQTSNFRVQPTESIVRFPNITNLCPFGEVENATRFASVYAWNRKRISNCVADYSVLYNSA
SFSTFKCYGVSPTKLNDLCFTNVYADSFVIRGDEVRQIAPGQTGKIADYNYKLPDDFTGCVI
AWNSNNLDSKVGGNYNYLYRLFRKSNLKPFERDISTEIYQAGSTPCNGVEGENCYFPLQSYG
FQPTNGVGYQPYRVVVLSFELLHAPATVCGPKKSTNLVKNKCVNFNFNGLTGTGVLTESNKK
FLPFQQFGRDIADTTDAVRDPQTLEILDITPCSFGGVSVITPGTNTSNQVAVLYQDVNCTEV
PVAIHADQLTPTWRVYSTGSNVFQTRAGCLIGAEHVNNSYECDIPIGAGICASYQTQTNSPG
SASSVASQSIIAYTMSLGAENSVAYSNNSIAIPTNFTISVTTEILPVSMTKTSVDCTMYICG
DSTECSNLLLQYGSFCTQLNRALTGIAVEQDKNTQEVFAQVKQIYKTPPIKDFGGENFSQIL
PDPSKPSKRSPIEDLLENKVTLADAGFIKQYGDCLGDIAARDLICAQKENGLTVLPPLLTDE
MIAQYTSALLAGTITSGWTFGAGPALQIPFPMQMAYRENGIGVTQNVLYENQKLIANQFNSA
IGKIQDSLSSTPSALGKLQDVVNQNAQALNTLVKQLSSNFGAISSVLNDILSRLDPPEAEVQ
IDRLITGRLQSLQTYVTQQLIRAAEIRASANLAATKMSECVLGQSKRVDFCGKGYHLMSFPQ
SAPHGVVFLHVTYVPAQEKNFTTAPAICHDGKAHFPREGVFVSNGTHWFVTQRNFYEPQIIT
TDNTFVSGNCDVVIGIVNNTVYDPLQPELDSGGDIIKLLNEQVNKEMQSSNLYMSMSSWCYT
HSLDGAGLFLFDHAAEEYEHAKKLIIFLNENNVPVQLNSTSAPEHKFEGLTQIFQKAYEHEQ
HISESINNIVDHAIKSKDHATFNFLQWYVAEQHEEEVLFKDILDKIELIGNENHGLYLADQY
VKGIAKSRKS
SpikeHexaProΔC ferritin with artificial glycosylation
site variant 3 (“HexaPro ΔC Gly 3 ferritin”)-
SEQ ID NO: 24
MFVFLVLLPLVSSQCVNLTTRTQLPPAYTNSFTRGVYYPDKVFRSSVLHSTQDLFLPFFSNV
TWFHAIHVSGTNGTKRFDNPVLPENDGVYFASTEKSNIIRGWIFGTTLDSKTQSLLIVNNAT
NVVIKVCEFQFCNDPFLGVYYHKNNKSWMESEFRVYSSANNCTFEYVSQPFLMDLEGKQGNF
KNLREFVFKNIDGYFKIYSKHTPINLVRDLPQGFSALEPLVDLPIGINITRFQTLLALHRSY
LTPGDSSSGWTAGAAAYYVGYLQPRTELLKYNENGTITDAVDCALDPLSETKCTLKSFTVEK
GIYQTSNERVQPTESIVRFPNITNLCPFGEVENATRFASVYAWNRKRISNCVADYSVLYNSA
SFSTFKCYGVSPTKLNDLCFTNVYADSFVIRGDEVRQIAPGQTGKIADYNYKLPDDFTGCVI
AWNSNNLDSKVGGNYNYLYRLERKSNLKPFERDISTEIYQAGSTPCNGVEGENCYFPLQSYG
FQPTNGVGYQPYRVVVLSFELLHAPATVCGPKKSTNLVKNKCVNFNFNGLTGTGVLTESNKK
FLPFQQFGRDIADTTDAVRDPQTLEILDITPCSFGGVSVITPGTNTSNQVAVLYQDVNCTEV
PVAIHADQLTPTWRVYSTGSNVFQTRAGCLIGAEHVNNSYECDIPIGAGICASYQTQTNSPG
SASSVASQSIIAYTMSLGAENSVAYSNNSIAIPTNFTISVTTEILPVSMTKTSVDCTMYICG
DSTECSNLLLQYGSFCTQLNRALTGIAVEQDKNTQEVFAQVKQIYKTPPIKDFGGENFSQIL
PDPSKPSKRSPIEDLLFNKVTLADAGFIKQYGDCLGDIAARDLICAQKENGLTVLPPLLTDE
MIAQYTSALLAGTITSGWTFGAGPALQIPFPMQMAYRENGIGVTQNVLYENQKLIANQFNSA
IGKIQDSLSSTPSALGKLQDVVNQNAQALNTLVKQLSSNFGAISSVLNDILSRLDPPEAEVQ
IDRLITGRLQSLQTYVTQQLIRAAEIRASANLAATKMSECVLGQSKRVDFCGKGYHLMSFPQ
SAPHGVVFLHVTYVPAQEKNFTTAPAICHDGKAHFPREGVFVSNGTHWFVTQRNFYEPQIIT
TDNTFVSGNCDVVIGIVNNTVYDPLQPELDSGGDIIKLLNEQVNKEMQSSNLYMSMSSWCYT
HSLDGAGLFLFDHAAEEYEHAKKLIIFLNENNVPVQLTSISAPENKTEGLTQIFQKAYEHEQ
HISESINNIVDHAIKSKDHATFNFLQWYVAEQHEEEVLFKDILDKIELIGNENHGLYLADQY
VKGIAKSRKS
SpikeHexaProΔC ferritin with artificial glycosylation
site variant 4 (“HexaPro ΔC Gly 4 ferritin”)-
SEQ ID NO: 25
MFVFLVLLPLVSSQCVNLTTRTQLPPAYTNSFTRGVYYPDKVFRSSVLHSTQDLFLPFFSNV
TWFHAIHVSGTNGTKRFDNPVLPENDGVYFASTEKSNIIRGWIFGTTLDSKTQSLLIVNNAT
NVVIKVCEFQFCNDPFLGVYYHKNNKSWMESEFRVYSSANNCTFEYVSQPFLMDLEGKQGNE
KNLREFVFKNIDGYFKIYSKHTPINLVRDLPQGFSALEPLVDLPIGINITRFQTLLALHRSY
LTPGDSSSGWTAGAAAYYVGYLQPRTFLLKYNENGTITDAVDCALDPLSETKCTLKSFTVEK
GIYQTSNFRVQPTESIVRFPNITNLCPFGEVENATRFASVYAWNRKRISNCVADYSVLYNSA
SFSTFKCYGVSPTKLNDLCFTNVYADSFVIRGDEVRQIAPGQTGKIADYNYKLPDDFTGCVI
AWNSNNLDSKVGGNYNYLYRLFRKSNLKPFERDISTEIYQAGSTPCNGVEGENCYFPLQSYG
FQPTNGVGYQPYRVVVLSFELLHAPATVCGPKKSTNLVKNKCVNFNFNGLTGTGVLTESNKK
FLPFQQFGRDIADTTDAVRDPQTLEILDITPCSFGGVSVITPGTNTSNQVAVLYQDVNCTEV
PVAIHADQLTPTWRVYSTGSNVFQTRAGCLIGAEHVNNSYECDIPIGAGICASYQTQTNSPG
SASSVASQSIIAYTMSLGAENSVAYSNNSIAIPTNFTISVTTEILPVSMTKTSVDCTMYICG
DSTECSNLLLQYGSFCTQLNRALTGIAVEQDKNTQEVFAQVKQIYKTPPIKDFGGFNFSQIL
PDPSKPSKRSPIEDLLENKVTLADAGFIKQYGDCLGDIAARDLICAQKENGLTVLPPLLTDE
MIAQYTSALLAGTITSGWTFGAGPALQIPFPMQMAYRENGIGVTQNVLYENQKLIANQFNSA
IGKIQDSLSSTPSALGKLQDVVNQNAQALNTLVKQLSSNFGAISSVLNDILSRLDPPEAEVQ
IDRLITGRLQSLQTYVTQQLIRAAEIRASANLAATKMSECVLGQSKRVDFCGKGYHLMSFPQ
SAPHGVVFLHVTYVPAQEKNFTTAPAICHDGKAHFPREGVFVSNGTHWFVTQRNFYEPQIIT
TDNTFVSGNCDVVIGIVNNTVYDPLQPELDSGGDIIKLLNEQVNKEMQSSNLYMSMSSWCYT
HSLDGAGLFLFDHAAEEYEHAKKLIIFLNENNVPVQLTSISAPEHKFEGLTQIFQKAYEHEQ
HISESINNIVDHAIKSKDHATFNFLQWYVAEQHEEEVLFKDILDKIELIGNNNTGLYLADQY
VKGIAKSRKS

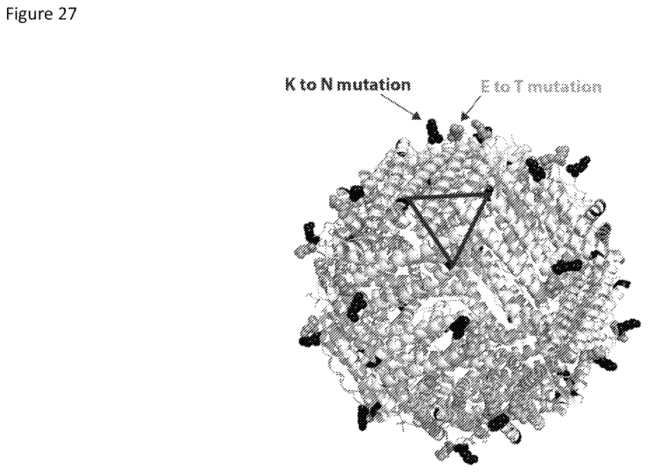

In SEQ ID NO:22, the two amino acid substitutions are K to N at a position corresponding to position 75 of SEQ ID NO:2, and E to T at a position corresponding to position 77 of SEQ ID NO:2. In SEQ ID NO:23, the two amino acid substitutions are T to N at a position corresponding to position 67 of SEQ ID NO:2, and I to T at a position corresponding to position 69 of SEQ ID NO:2. In SEQ ID NO:24, the two amino acid substitutions are H to N at a position corresponding to position 74 of SEQ ID NO:2, and F to T at a position corresponding to position 76 of SEQ ID NO:2. In SEQ ID NO:25, the two amino acid substitutions are E to N at a position corresponding to position 143 of SEQ ID NO:2, and H to T at a position corresponding to position 145 of SEQ ID NO:2. FIG. 27 schematically illustrates the position of the engineered glycosylation site in a SARS-CoV-2 Spike fusion protein nanoparticle formed from SEQ ID NO:22.

### Example 14: Testing of SARS-CoV-2 Spike Protein Antigens Based on of Naturally Occurring Variants of Coronavirus Spike Protein

Testing was conducted of SARS-CoV-2 Spike protein antigens based on naturally occurring variants of coronavirus Spike protein. Coronavirus Spike protein variants were selected for the study from five naturally circulating SARS-CoV-2 variants: D614G, B.1.1.7, B.1.429 (also known as “LA variant”), P1, and B.1.351, which, among others, were deemed “variants of concern” by Centers for Disease Control and Prevention of the U.S. Department of Health and Human Services. The amino acid sequences of the fusion proteins based on these SARS-CoV-2 Spike protein variants (“variant SARS-CoV-2 Spike protein antigens”) are shown below as SEQ ID NO:26 (based on D614G), SEQ ID NO:27 (based on B.1.1.7), SEQ ID NO:28 (based on B.1.351), SEQ ID NO:29 (based on B.1.429), and SEQ ID NO:30 (based on P1). SARS-CoV-2 Spike signal peptide sequences are shown in bold/underlined font, Ser/Gly linker regions are underlined, H. pylori ferritin sequences are italicized and underlined, amino acid substitutions within the Spike domain in comparison to SEQ ID NO:2 (also summarized in Table 1) are shown in bold, and deletions are shown with an underscore symbol.

Spike HexaProΔC ferritin D614G (“HexaPro ΔC ferritin D614G”)-
SEQ ID NO: 26
MFVFLVLLPLVSSQCVNLTTRTQLPPAYTNSFTRGVYYPDKVFRSSVLHSTQDLFLPFFSNV
TWFHAIHVSGTNGTKRFDNPVLPENDGVYFASTEKSNIIRGWIFGTTLDSKTQSLLIVNNAT
NVVIKVCEFQFCNDPFLGVYYHKNNKSWMESEFRVYSSANNCTFEYVSQPFLMDLEGKQGNE
KNLREFVFKNIDGYFKIYSKHTPINLVRDLPQGFSALEPLVDLPIGINITRFQTLLALHRSY
LTPGDSSSGWTAGAAAYYVGYLQPRTFLLKYNENGTITDAVDCALDPLSETKCTLKSFTVEK
GIYQTSNFRVQPTESIVRFPNITNLCPFGEVENATRFASVYAWNRKRISNCVADYSVLYNSA
SFSTFKCYGVSPTKLNDLCFTNVYADSFVIRGDEVRQIAPGQTGKIADYNYKLPDDFTGCVI
AWNSNNLDSKVGGNYNYLYRLFRKSNLKPFERDISTEIYQAGSTPCNGVEGENCYFPLQSYG
FQPTNGVGYQPYRVVVLSFELLHAPATVCGPKKSTNLVKNKCVNFNFNGLTGTGVLTESNKK
FLPFQQFGRDIADTTDAVRDPQTLEILDITPCSFGGVSVITPGTNTSNQVAVLYQGVNCTEV
PVAIHADQLTPTWRVYSTGSNVFQTRAGCLIGAEHVNNSYECDIPIGAGICASYQTQTNSPG
SASSVASQSIIAYTMSLGAENSVAYSNNSIAIPTNFTISVTTEILPVSMTKTSVDCTMYICG
DSTECSNLLLQYGSFCTQLNRALTGIAVEQDKNTQEVFAQVKQIYKTPPIKDFGGENFSQIL
PDPSKPSKRSPIEDLLENKVTLADAGFIKQYGDCLGDIAARDLICAQKENGLTVLPPLLTDE
MIAQYTSALLAGTITSGWTFGAGPALQIPFPMQMAYRENGIGVTQNVLYENQKLIANQENSA
IGKIQDSLSSTPSALGKLQDVVNQNAQALNTLVKQLSSNFGAISSVLNDILSRLDPPEAEVQ
IDRLITGRLQSLQTYVTQQLIRAAEIRASANLAATKMSECVLGQSKRVDFCGKGYHLMSFPQ
SAPHGVVFLHVTYVPAQEKNFTTAPAICHDGKAHFPREGVFVSNGTHWFVTQRNFYEPQIIT
TDNTFVSGNCDVVIGIVNNTVYDPLQPELDSGGDIIKLLNEQVNKEMQSSNLYMSMSSWCYT
HSLDGAGLFLFDHAAEEYEHAKKLIIFLNENNVPVQLTSISAPEHKFEGLTQIFQKAYEHEQ
HISESINNIVDHAIKSKDHATFNFLQWYVAEQHEEEVLFKDILDKIELIGNENHGLYLADQY
VKGIAKSRKS
SpikeHexaProΔC ferritin B.1.1.7 (“HexaPro ΔC ferritin B.1.1.7”)--
SEQ ID NO: 27
MFVFLVLLPLVSSQCVNLTTRTQLPPAYTNSFTRGVYYPDKVFRSSVLHSTQDLFLPFFSNV
TWFHAI__SGTNGTKRFDNPVLPFNDGVYFASTEKSNIIRGWIFGTTLDSKTQSLLIVNNAT
NVVIKVCEFQFCNDPFLGV_YHKNNKSWMESEFRVYSSANNCTFEYVSQPFLMDLEGKQGNE
KNLREFVFKNIDGYFKIYSKHTPINLVRDLPQGFSALEPLVDLPIGINITRFQTLLALHRSY
LTPGDSSSGWTAGAAAYYVGYLQPRTELLKYNENGTITDAVDCALDPLSETKCTLKSFTVEK
GIYQTSNERVQPTESIVRFPNITNLCPFGEVENATRFASVYAWNRKRISNCVADYSVLYNSA
SFSTFKCYGVSPTKLNDLCFTNVYADSFVIRGDEVRQIAPGQTGKIADYNYKLPDDFTGCVI
AWNSNNLDSKVGGNYNYLYRLFRKSNLKPFERDISTEIYQAGSTPCNGVEGENCYFPLQSYG
FQPTYGVGYQPYRVVVLSFELLHAPATVCGPKKSTNLVKNKCVNFNFNGLTGTGVLTESNKK
FLPFQQFGRDIDDTTDAVRDPQTLEILDITPCSFGGVSVITPGTNTSNQVAVLYQGVNCTEV
PVAIHADQLTPTWRVYSTGSNVFQTRAGCLIGAEHVNNSYECDIPIGAGICASYQTQTNSHG
SASSVASQSIIAYTMSLGAENSVAYSNNSIAIPINFTISVTTEILPVSMTKTSVDCTMYICG
DSTECSNLLLQYGSFCTQLNRALTGIAVEQDKNTQEVFAQVKQIYKTPPIKDFGGENFSQIL
PDPSKPSKRSPIEDLLENKVTLADAGFIKQYGDCLGDIAARDLICAQKENGLTVLPPLLTDE
MIAQYTSALLAGTITSGWTFGAGPALQIPFPMQMAYRENGIGVTQNVLYENQKLIANQENSA
IGKIQDSLSSTPSALGKLQDVVNQNAQALNTLVKQLSSNFGAISSVLNDILARLDPPEAEVQ
IDRLITGRLQSLQTYVTQQLIRAAEIRASANLAATKMSECVLGQSKRVDFCGKGYHLMSFPQ
SAPHGVVFLHVTYVPAQEKNFTTAPAICHDGKAHFPREGVFVSNGTHWFVTQRNFYEPQIIT
THNTFVSGNCDVVIGIVNNTVYDPLQPELDSGGDIIKLLNEQVNKEMQSSNLYMSMSSWCYT
HSLDGAGLFLFDHAAEEYEHAKKLIIFLNENNVPVQLTSISAPEHKFEGLTQIFQKAYEHEQ
HISESINNIVDHAIKSKDHATFNFLQWYVAEQHEEEVLFKDILDKIELIGNENHGLYLADQY
VKGIAKSRKS
SpikeHexaProΔC ferritin B.1.351 (“HexaPro ΔC ferritin B.1.351”)-
SEQ ID NO: 28
MFVFLVLLPLVSSQCVNFTTRTQLPPAYTNSFTRGVYYPDKVFRSSVLHSTQDLFLPFFSNV
TWFHAIHVSGTNGTKRFANPVLPFNDGVYFASTEKSNIIRGWIFGTTLDSKTQSLLIVNNAT
NVVIKVCEFQFCNDPFLGVYYHKNNKSWMESEFRVYSSANNCTFEYVSQPFLMDLEGKQGNE
KNLREFVFKNIDGYFKIYSKHTPINLVRGLPQGFSALEPLVDLPIGINITRFQTL___HISY
LTPGDSSSGWTAGAAAYYVGYLQPRTFLLKYNENGTITDAVDCALDPLSETKCTLKSFTVEK
GIYQTSNERVQPTESIVRFPNITNLCPFGEVENATRFASVYAWNRKRISNCVADYSVLYNSA
SFSTFKCYGVSPTKLNDLCFTNVYADSFVIRGDEVRQIAPGQTGNIADYNYKLPDDFTGCVI
AWNSNNLDSKVGGNYNYLYRLFRKSNLKPFERDISTEIYQAGSTPCNGVKGENCYFPLQSYG
FQPTYGVGYQPYRVVVLSFELLHAPATVCGPKKSTNLVKNKCVNFNFNGLTGTGVLTESNKK
FLPFQQFGRDIADTTDAVRDPQTLEILDITPCSFGGVSVITPGTNTSNQVAVLYQGVNCTEV
PVAIHADQLTPTWRVYSTGSNVFQTRAGCLIGAEHVNNSYECDIPIGAGICASYQTQTNSPG
SASSVASQSIIAYTMSLGVENSVAYSNNSIAIPTNFTISVTTEILPVSMTKTSVDCTMYICG
DSTECSNLLLQYGSFCTQLNRALTGIAVEQDKNTQEVFAQVKQIYKTPPIKDFGGFNFSQIL
PDPSKPSKRSPIEDLLENKVTLADAGFIKQYGDCLGDIAARDLICAQKENGLTVLPPLLTDE
MIAQYTSALLAGTITSGWTFGAGPALQIPFPMQMAYRENGIGVTQNVLYENQKLIANQFNSA
IGKIQDSLSSTPSALGKLQDVVNQNAQALNTLVKQLSSNFGAISSVLNDILSRLDPPEAEVQ
IDRLITGRLQSLQTYVTQQLIRAAEIRASANLAATKMSECVLGQSKRVDFCGKGYHLMSFPQ
SAPHGVVFLHVTYVPAQEKNFTTAPAICHDGKAHFPREGVFVSNGTHWFVTQRNFYEPQIIT
TDNTFVSGNCDVVIGIVNNTVYDPLQPELDSGGDIIKLLNEQVNKEMQSSNLYMSMSSWCYT
HSLDGAGLFLFDHAAEEYEHAKKLIIFLNENNVPVQLTSISAPEHKFEGLTQIFQKAYEHEQ
HISESINNIVDHAIKSKDHATFNFLQWYVAEQHEEEVLFKDILDKIELIGNENHGLYLADQY
VKGIAKSRKS
SpikeHexaProΔC ferritin B.1.429 (“HexaPro ΔC ferritin B.1.429”)-
SEQ ID NO: 29
MFVFLVLLPLVSIQCVNLTTRTQLPPAYTNSFTRGVYYPDKVFRSSVLHSTQDLFLPFFSNV
TWFHAIHVSGTNGTKRFDNPVLPENDGVYFASTEKSNIIRGWIFGTTLDSKTQSLLIVNNAT
NVVIKVCEFQFCNDPFLGVYYHKNNKSCMESEFRVYSSANNCTFEYVSQPFLMDLEGKQGNF
KNLREFVFKNIDGYFKIYSKHTPINLVRDLPQGFSALEPLVDLPIGINITRFQTLLALHRSY
LTPGDSSSGWTAGAAAYYVGYLQPRTELLKYNENGTITDAVDCALDPLSETKCTLKSFTVEK
GIYQTSNERVQPTESIVRFPNITNLCPFGEVENATRFASVYAWNRKRISNCVADYSVLYNSA
SFSTFKCYGVSPTKLNDLCFTNVYADSFVIRGDEVRQIAPGQTGKIADYNYKLPDDFTGCVI
AWNSNNLDSKVGGNYNYRYRLFRKSNLKPFERDISTEIYQAGSTPCNGVEGENCYFPLQSYG
FQPTNGVGYQPYRVVVLSFELLHAPATVCGPKKSTNLVKNKCVNFNENGLTGTGVLTESNKK
FLPFQQFGRDIADTTDAVRDPQTLEILDITPCSFGGVSVITPGTNTSNQVAVLYQGVNCTEV
PVAIHADQLTPTWRVYSTGSNVFQTRAGCLIGAEHVNNSYECDIPIGAGICASYQTQTNSPG
SASSVASQSIIAYTMSLGAENSVAYSNNSIAIPTNFTISVTTEILPVSMTKTSVDCTMYICG
DSTECSNLLLQYGSFCTQLNRALTGIAVEQDKNTQEVFAQVKQIYKTPPIKDFGGFNFSQIL
PDPSKPSKRSPIEDLLENKVTLADAGFIKQYGDCLGDIAARDLICAQKENGLTVLPPLLTDE
MIAQYTSALLAGTITSGWTFGAGPALQIPFPMQMAYRENGIGVTQNVLYENQKLIANQFNSA
IGKIQDSLSSTPSALGKLQDVVNQNAQALNTLVKQLSSNFGAISSVLNDILSRLDPPEAEVQ
IDRLITGRLQSLQTYVTQQLIRAAEIRASANLAATKMSECVLGQSKRVDFCGKGYHLMSFPQ
SAPHGVVFLHVTYVPAQEKNFTTAPAICHDGKAHFPREGVFVSNGTHWFVTQRNFYEPQIIT
TDNTFVSGNCDVVIGIVNNTVYDPLQPELDSGGDIIKLLNEQVNKEMQSSNLYMSMSSWCYT
HSLDGAGLFLFDHAAEEYEHAKKLIIFLNENNVPVQLTSISAPEHKFEGLTQIFQKAYEHEQ
HISESINNIVDHAIKSKDHATFNFLQWYVAEQHEEEVLFKDILDKIELIGNENHGLYLADQY
VKGIAKSRKS
SpikeHexaProΔC ferritin P1 (“HexaPro ΔC ferritin P1”)-
SEQ ID NO: 30
MFVFLVLLPLVSSQCVNFTNRTQLPSAYTNSFTRGVYYPDKVFRSSVLHSTQDLFLPFFSNV
TWFHAIHVSGTNGTKRFDNPVLPENDGVYFASTEKSNIIRGWIFGTTLDSKTQSLLIVNNAT
NVVIKVCEFQFCNYPFLGVYYHKNNKSWMESEFRVYSSANNCTFEYVSQPFLMDLEGKQGNF
KNLSEFVFKNIDGYFKIYSKHTPINLVRDLPQGFSALEPLVDLPIGINITRFQTLLALHRSY
LTPGDSSSGWTAGAAAYYVGYLQPRTELLKYNENGTITDAVDCALDPLSETKCTLKSFTVEK
GIYQTSNERVQPTESIVRFPNITNLCPFGEVENATRFASVYAWNRKRISNCVADYSVLYNSA
SFSTFKCYGVSPTKLNDLCFTNVYADSFVIRGDEVRQIAPGQTGTIADYNYKLPDDFTGCVI
AWNSNNLDSKVGGNYNYLYRLFRKSNLKPFERDISTEIYQAGSTPCNGVKGENCYFPLQSYG
FQPTYGVGYQPYRVVVLSFELLHAPATVCGPKKSTNLVKNKCVNFNFNGLTGTGVLTESNKK
FLPFQQFGRDIADTTDAVRDPQTLEILDITPCSFGGVSVITPGTNTSNQVAVLYQGVNCTEV
PVAIHADQLTPTWRVYSTGSNVFQTRAGCLIGAEYVNNSYECDIPIGAGICASYQTQTNSPG
SASSVASQSIIAYTMSLGAENSVAYSNNSIAIPTNFTISVTTEILPVSMTKTSVDCTMYICG
DSTECSNLLLQYGSFCTQLNRALTGIAVEQDKNTQEVFAQVKQIYKTPPIKDFGGENFSQIL
PDPSKPSKRSPIEDLLENKVTLADAGFIKQYGDCLGDIAARDLICAQKENGLTVLPPLLTDE
MIAQYTSALLAGTITSGWTFGAGPALQIPFPMQMAYRENGIGVTQNVLYENQKLIANQENSA
IGKIQDSLSSTPSALGKLQDVVNQNAQALNTLVKQLSSNFGAISSVLNDILSRLDPPEAEVQ
IDRLITGRLQSLQTYVTQQLIRAAEIRASANLAAIKMSECVLGQSKRVDFCGKGYHLMSFPQ
SAPHGVVFLHVTYVPAQEKNFTTAPAICHDGKAHFPREGVFVSNGTHWFVTQRNFYEPQIIT
TDNTFVSGNCDVVIGIVNNTVYDPLQPELDSGGDIIKLLNEQVNKEMQSSNLYMSMSSWCYT
HSLDGAGLFLFDHAAEEYEHAKKLIIFLNENNVPVQLTSISAPEHKFEGLTQIFQKAYEHEQ
HISESINNIVDHAIKSKDHATFNFLQWYVAEQHEEEVLFKDILDKIELIGNENHGLYLADQY
VKGIAKSRKS

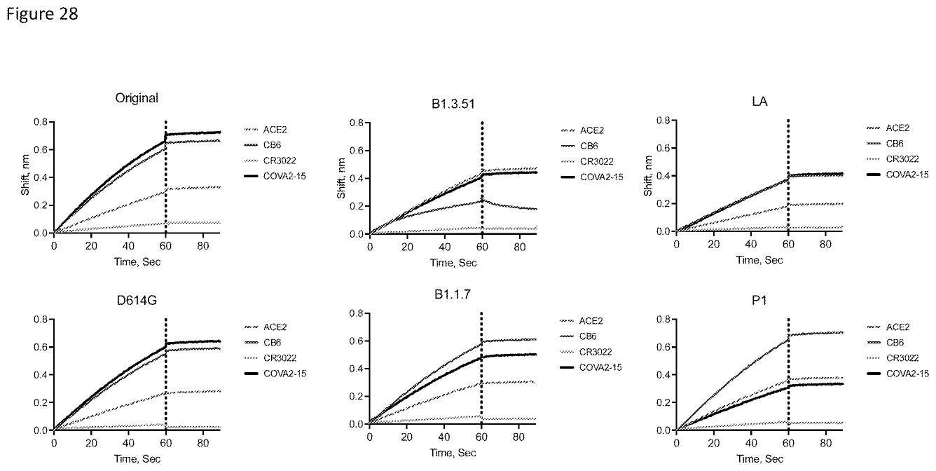

Expression and purification of the above SARS-CoV-2 Spike protein antigens based on naturally occurring variants of coronavirus Spike protein was performed substantially as described in Example 10. Protein samples were flash frozen in PBS with 10% sucrose for storage. BLI was used to check the binding of SARS-CoV-2 Spike protein antigens to conformational mAbs and to ACE2 receptor. The BLI experiments were conducted substantially as described in Example 11. The results are summarized in FIG. 28. Equivalent binding of SpikeHexaProAC ferritin and each of the five variant SARS-CoV-2 Spike protein antigens to conformational antibodies and ACE2 receptor was observed.

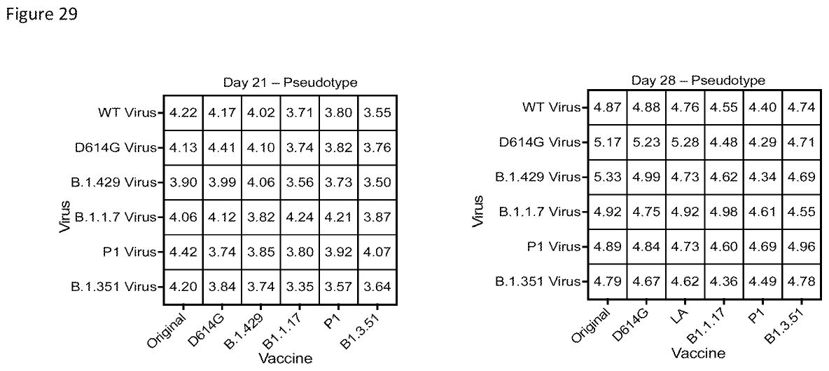

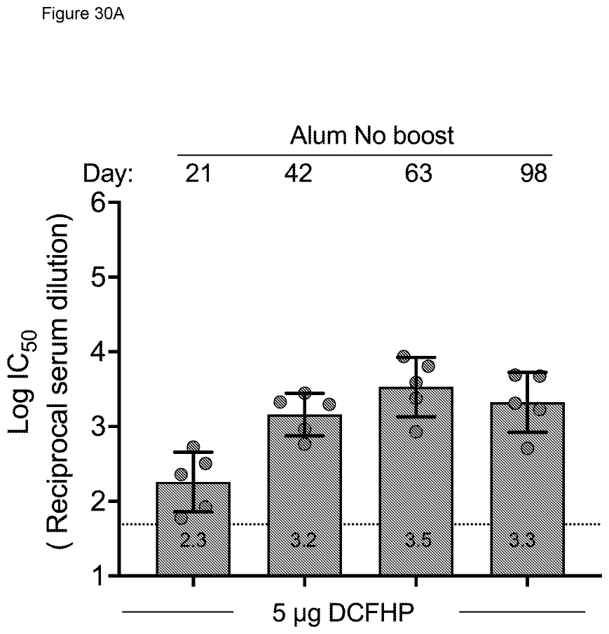

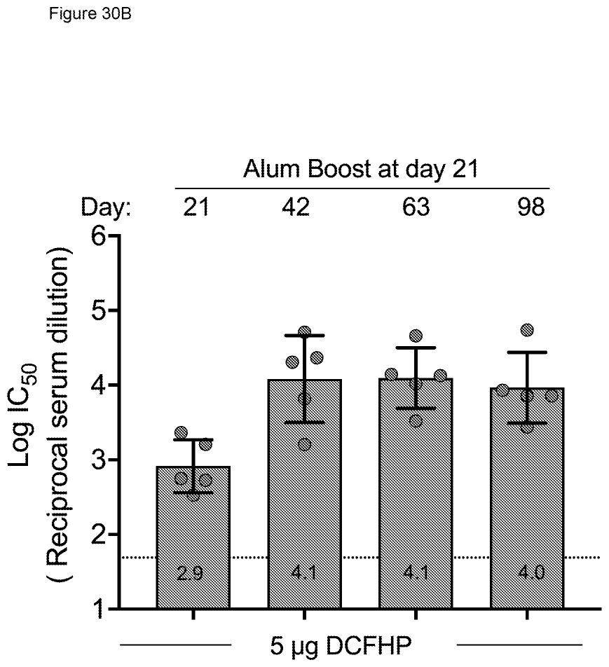

Testing of neutralizing antibody responses elicited by variant SARS-CoV-2 Spike protein antigens was conducted. Five mice per groups were immunized with each of variant SARS-CoV-2 Spike protein antigens and SpikeHexaProAC ferritin (SEQ ID NO:16). The immunization was conducted substantially as described in Example 11. The blood samples were drawn on “Day 0” (prior to immunization), “Day 21,” and “Day 28” The neutralization titers of the sera collected from the experimental animals were assessed using SARS-CoV-2 Spike pseudotyped lentivirus neutralization assay described in Example 1 against the panel of six pseudoviruses (Wuhan-1, D614G, B.1.429, B1.1.7, P1, and B.1.351). The results are summarized in 36 IC50 values were generated from using SARS-CoV-2 Spike pseudotyped lentivirus neutralization assay with pooled serum from “Day 21,” and another 36 values from the pooled serum at “Day 28.” The results are summarized as a “heat map” shown in the tables in FIG. 29. Each value shown in tables is a log₁₀IC50 value of the pooled serum from the mice immunized with the same SARS-CoV-2 Spike protein antigen against a specific pseudotyped virus. The analysis summarized in FIG. 29 allowed for comparison of neutralizing activity of each SARS-CoV-2 Spike protein antigen against each virus variant. The animals immunized with SpikeHexaProAC ferritin version of the SARS-CoV-2 Spike protein antigen had the highest neutralization titers across the panel of the tested pseudoviruses.

### Example 15: Adjuvant Testing

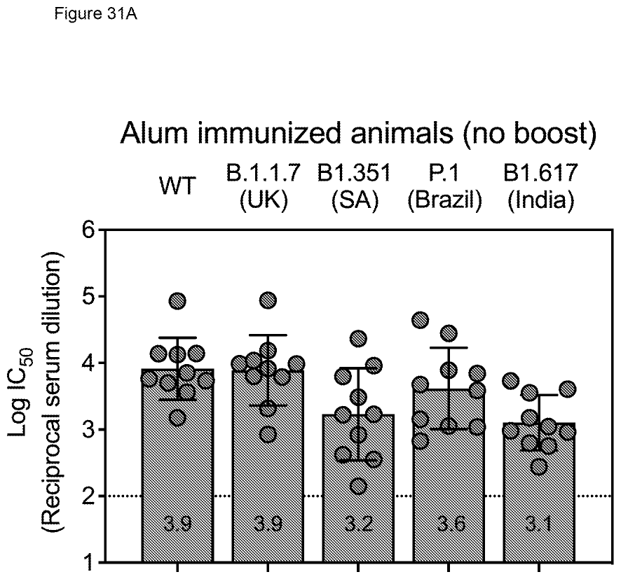

Adjuvant testing was conducted by testing SARS-CoV-2 neutralization response in mice immunized with adjuvanted SpikeHexaProAC ferritin (SEQ ID NO:16). The results are illustrated in FIGS. 30A-34F. FIGS. 30A and 30B illustrate the results of the experimental testing of neutralization responses against wild type SARS-CoV-2 in mice immunized with SpikeHexaProAC ferritin adjuvanted with 500 μg alum. Groups of 5 mice were immunized with 5 μg of SpikeHexaProAC ferritin adjuvanted with 500 μg alum (Alhydrogel®, InvivoGen, San Diego, California) via subcutaneous injections. The first group (FIG. 30A) was immunized once, and the second group (FIG. 30B) was boosted 21 days after the initial immunization. Mice were bled at the indicated time points to monitor immune response, and, subsequently, wild type SARS-CoV-2 pseudo-virus neutralization titers were measured substantially as discussed elsewhere in the present disclosure. Briefly, diluted mouse serum samples were incubated with pseudo-typed SARS-CoV-2 virus for 1 hour and added onto HeLa cells expressing ACE2 and TMPRSS 2. The infectivity of the cells were measured after 48 hours by measuring luciferase enzyme activity. The relative luciferase enzyme activity was plotted against the serum dilution. and the 50% infective concentrations (IC50) were calculated from the dilution curves. The experiments showed that a single dose immunization with SpikeHexaProAC ferritin adjuvanted with alum induced SARS-CoV-2 neutralization response in mice. While a boost at day 21 improved the neutralization response, a single-dose immunization with SpikeHexaProAC ferritin adjuvanted with alum was sufficient to generate adequate immune response against SARS-CoV-2.

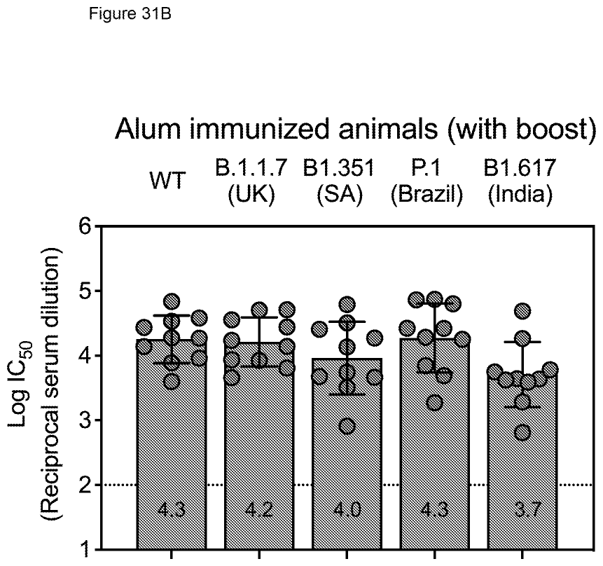

FIGS. 31A and 31B illustrate the results of the experimental testing of the neutralization responses against wild type SARS-CoV-2 and SARS-CoV-2 variants in mice immunized with SpikeHexaProAC ferritin adjuvanted with 500 μg alum. Groups of 10 mice were immunized with 5 μg of SpikeHexaProAC ferritin adjuvanted with 500 μg alum (Alhydrogel®, InvivoGen, San Diego, California) via subcutaneous injections. The first group (FIG. 31A) was immunized once, and the second group (FIG. 31B) was boosted 21 days after the initial immunization. Mice were bled 63 days after the initial immunization to monitor immune response. Subsequently, neutralizing titers of the serum samples were assayed against pseudo-typed wild type SARS-CoV-2 and SARS-CoV-2 variants substantially as discussed above and elsewhere in the present disclosure. The experiments showed that sera from mice immunized with single dose of SpikeHexaProAC ferritin adjuvanted with alum were able to neutralize both wild type SARS-CoV-2 and SARS-CoV-2 variants. While a boost at day 21 increased the neutralization activity against, a single dose immunization with SpikeHexaProAC ferritin advanced with alum was effective to mount SARS-CoV-2 antiviral response against all the variant tested, including B.1.617.2 (“delta variant”).

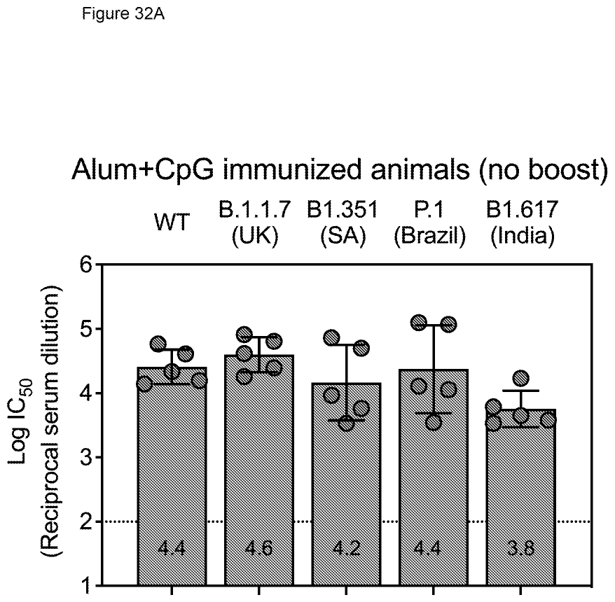

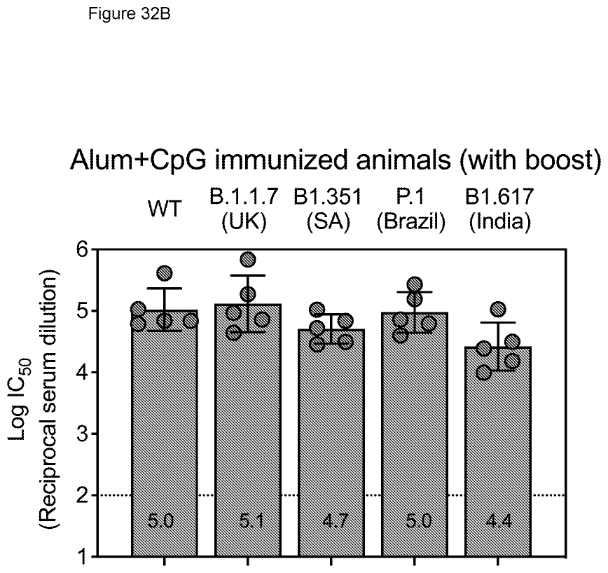

FIGS. 32A and 32B illustrate the results of the experimental testing of the neutralization responses against wild type SARS-CoV-2 and SARS-CoV-2 variants in mice immunized with SpikeHexaProAC ferritin adjuvanted with alum and CpG. Groups of 10 mice were immunized with 5 μg of SpikeHexaProAC ferritin adjuvanted with 500 μg alum (Alhydrogel®, InvivoGen, San Diego, California) and 20 μg of CpG (InvivoGen, San Diego, California) via subcutaneous injections. The first group (FIG. 32A) was immunized once, and the second group (FIG. 32B) was boosted 21 days after the initial immunization. Mice were bled 63 days after the initial immunization to monitor immune response. Subsequently, neutralizing titers of the serum samples were assayed against pseudo-typed wild type SARS-CoV-2 variants substantially as discussed above and elsewhere in the present disclosure. The experiments showed that a single dose of SpikeHexaProAC ferritin adjuvanted with alum and CpG induced strong neutralization response in mice against both wild type SARS-CoV-2 and SARS-CoV-2 variants. A boost at day 21 increased the neutralization activity. The experimental testing showed that inclusion of CpG as an adjuvant in addition to alum was beneficial in comparison to the use of alum alone.

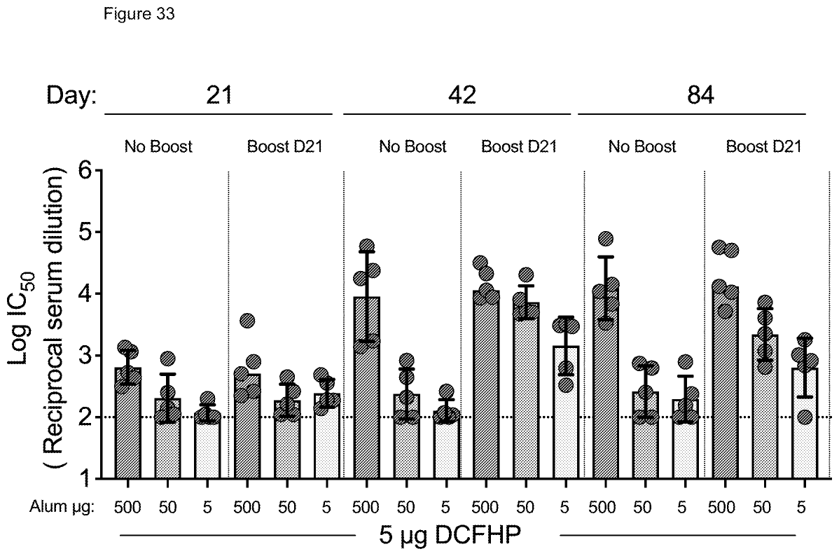

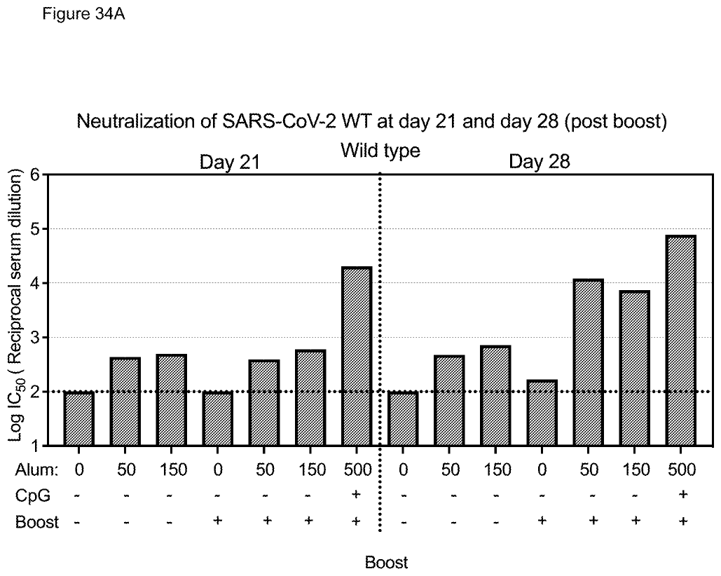

FIG. 33 illustrates the results of the experimental testing of the neutralization responses against wild type SARS-CoV-2 in mice immunized with SpikeHexaProAC ferritin adjuvanted with different doses of alum (Alhydrogel®, InvivoGen, San Diego, California). Groups of 5 mice were immunized with 5 μg of SpikeHexaProAC ferritin adjuvanted with 500, 50, or 5 μg alum (Alhydrogel®, InvivoGen, San Diego, California) via subcutaneous injections. Mice were bled at different time points after the initial immunization to monitor immune response. Subsequently, neutralizing titers of the serum samples were assayed against pseudo-typed wild type SARS-CoV-2 variants substantially as discussed above and elsewhere in the present disclosure. The experiments showed that increasing doses of alum improved the immune response, and that, at lower doses of alum, a boost was beneficial. The experiments also showed that neutralization responses induced by single-dose SpikeHexaProAC immunization (no boost) adjuvanted with the highest tested dose of alum improved with time. With the highest tested dose of alum, single-dose neutralization responses measured at day 42 and day 84 were comparable to the neutralization response induced by a prime-boost regimen. Thus, a single dose immunization with SpikeHexaProAC adjuvanted with higher amounts of alum may be sufficient to mount anti-SARS-CoV-2 responses.

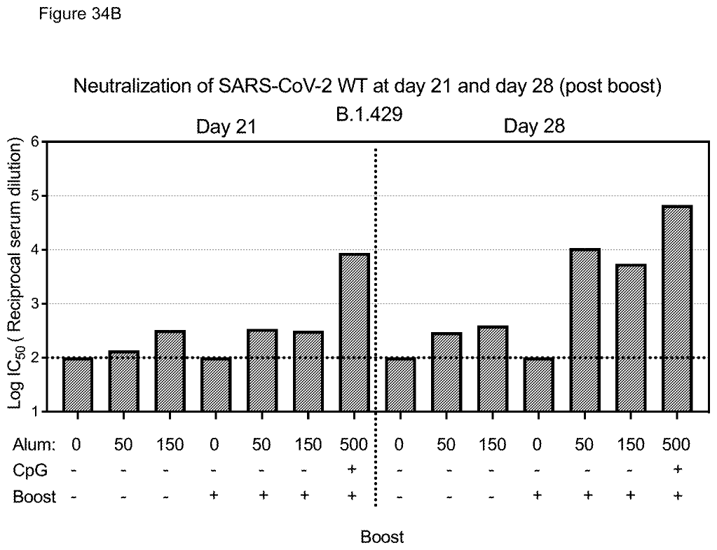

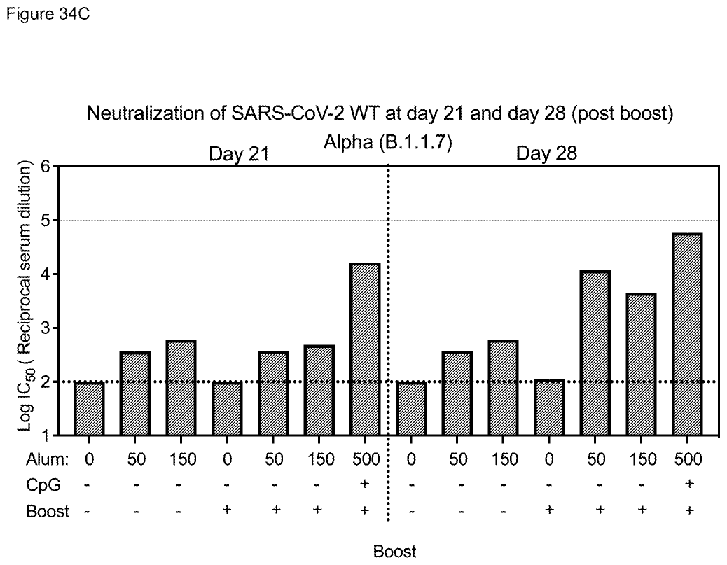

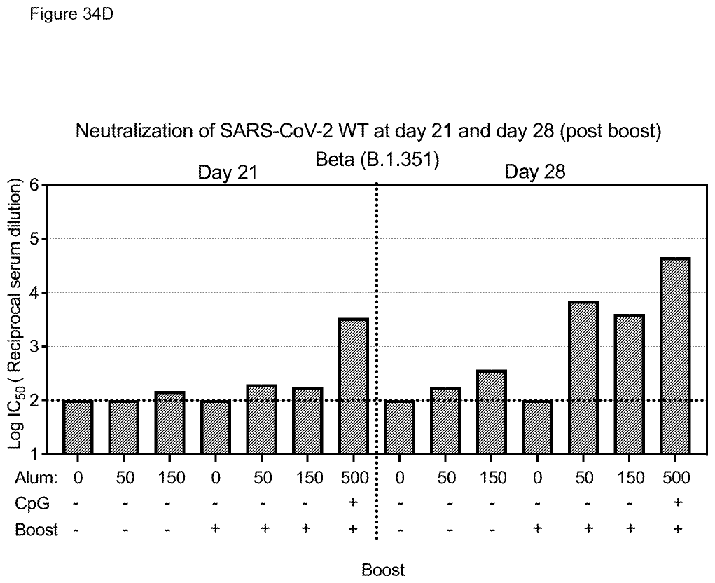

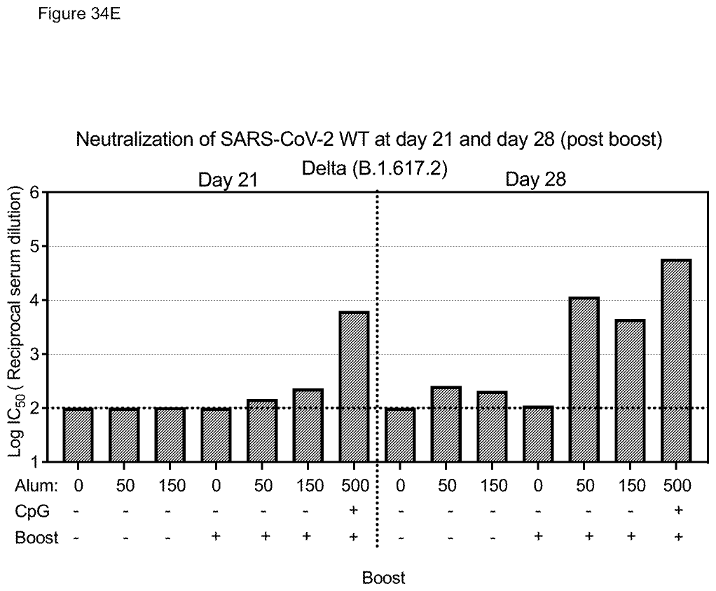

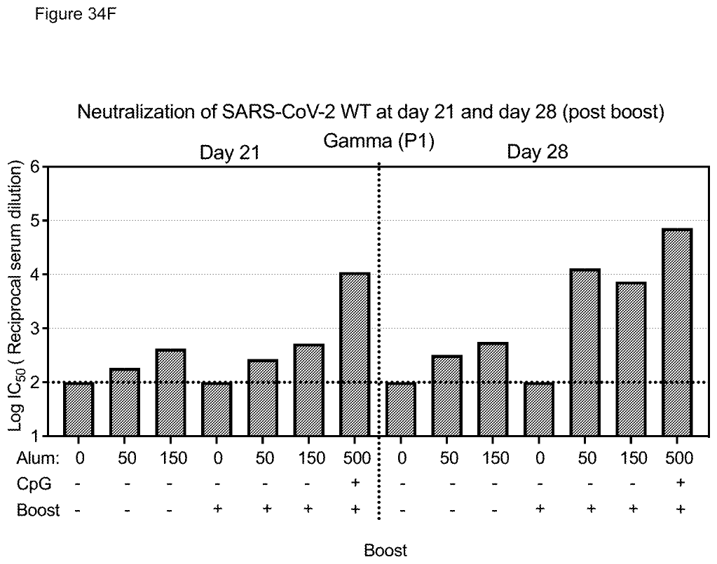

FIGS. 34A-34F illustrate the results of the experimental testing of the neutralization responses against wild type SARS-CoV-2 and SARS-CoV-2 variants in mice immunized with SpikeHexaProAC ferritin adjuvanted with different doses of alum (Alhydrogel®, InvivoGen, San Diego, California), either alone or in combination with 20 μg of CpG. Groups of 5 mice were immunized with 10 μg of SpikeHexaProAC ferritin adjuvanted with 500, 50, or 50 μg alum (Alhydrogel®, InvivoGen, San Diego, California) via subcutaneous injections. For each tested adjuvant, one group received single immunization, and a second group was boosted 21 days after the primary immunization. Mice were bled at day 21 and day 28 to monitor immune response. Serum samples from 5 mice of each group were pooled Subsequently, neutralizing titers of the pooled serum samples were assayed against pseudo-typed wild type SARS-CoV-2 variants substantially as discussed above and elsewhere in the present disclosure. The experiments showed immunization with SpikeHexaProAC adjuvanted with alum doses between 50 and 150 μg in prime-boost regimen induced adequate neutralization responses against both wild-type SARS-CoV-2 and SARS-CoV-2 variants, including B.1.617.2 (“delta variant”).

### Example 16: SpikeHexaProAC Ferritin Variations

Variations of SpikeHexaProAC ferritin that include different versions of a signal peptide sequence of SARS-CoV-2 protein are envisioned, with two examples shown below as SEQ ID NOs 33 and 34. SARS-CoV-2 Spike signal peptide sequences are shown in bold/underlined font, Ser/Gly linker regions are underlined, and H. pylori ferritin sequences are italicized and underlined.

SpikeHexaProΔC ferritin variation-
SEQ ID NO: 33
MFVFLVLLPLVSQCVNLTTRTQLPPAYTNSFTRGVYYPDKVERSSVLHSTQDLFLPFFSNVT
WFHAIHVSGTNGTKRFDNPVLPFNDGVYFASTEKSNIIRGWIFGTTLDSKTQSLLIVNNATN
VVIKVCEFQFCNDPFLGVYYHKNNKSWMESEFRVYSSANNCTFEYVSQPFLMDLEGKQGNEK
NLREFVFKNIDGYFKIYSKHTPINLVRDLPQGFSALEPLVDLPIGINITRFQTLLALHRSYL
TPGDSSSGWTAGAAAYYVGYLQPRTFLLKYNENGTITDAVDCALDPLSETKCTLKSFTVEKG
IYQTSNFRVQPTESIVRFPNITNLCPFGEVENATRFASVYAWNRKRISNCVADYSVLYNSAS
FSTFKCYGVSPTKLNDLCFTNVYADSFVIRGDEVRQIAPGQTGKIADYNYKLPDDFTGCVIA
WNSNNLDSKVGGNYNYLYRLFRKSNLKPFERDISTEIYQAGSTPCNGVEGENCYFPLQSYGF
QPTNGVGYQPYRVVVLSFELLHAPATVCGPKKSTNLVKNKCVNFNENGLTGTGVLTESNKKE
LPFQQFGRDIADTTDAVRDPQTLEILDITPCSFGGVSVITPGTNTSNQVAVLYQDVNCTEVP
VAIHADQLTPTWRVYSTGSNVFQTRAGCLIGAEHVNNSYECDIPIGAGICASYQTQTNSPGS
ASSVASQSIIAYTMSLGAENSVAYSNNSIAIPTNFTISVTTEILPVSMTKTSVDCTMYICGD
STECSNLLLQYGSFCTQLNRALTGIAVEQDKNTQEVFAQVKQIYKTPPIKDFGGENFSQILP
DPSKPSKRSPIEDLLFNKVTLADAGFIKQYGDCLGDIAARDLICAQKENGLTVLPPLLTDEM
IAQYTSALLAGTITSGWTFGAGPALQIPFPMQMAYRENGIGVTQNVLYENQKLIANQFNSAI
GKIQDSLSSTPSALGKLQDVVNQNAQALNTLVKQLSSNFGAISSVLNDILSRLDPPEAEVQI
DRLITGRLQSLQTYVTQQLIRAAEIRASANLAATKMSECVLGQSKRVDFCGKGYHLMSFPQS
APHGVVFLHVTYVPAQEKNFTTAPAICHDGKAHFPREGVFVSNGTHWFVTQRNFYEPQIITT
DNTFVSGNCDVVIGIVNNTVYDPLQPELDSGGDIIKLLNEQVNKEMQSSNLYMSMSSWCYTH
SLDGAGLFLFDHAAEEYEHAKKLIIFLNENNVPVQLTSISAPEHKFEGLTQIFQKAYEHEQH
ISESINNIVDHAIKSKDHATFNFLQWYVAEQHEEEVLENDILDKIELIGNENHGLYLADQYV
KGIAKSRKS
SpikeHexaProΔC ferritin variation-
SEQ ID NO: 34
MFVFLVLLPLVSSSQCVNLTTRTQLPPAYTNSFTRGVYYPDKVFRSSVLHSTQDLFLPFFSN
VTWFHAIHVSGTNGTKRFDNPVLPENDGVYFASTEKSNIIRGWIFGTTLDSKTQSLLIVNNA
TNVVIKVCEFQFCNDPFLGVYYHKNNKSWMESEFRVYSSANNCTFEYVSQPFLMDLEGKQGN
FKNLREFVFKNIDGYFKIYSKHTPINLVRDLPQGFSALEPLVDLPIGINITRFQTLLALHRS
YLTPGDSSSGWTAGAAAYYVGYLQPRTFLLKYNENGTITDAVDCALDPLSETKCTLKSFTVE
KGIYQTSNFRVQPTESIVRFPNITNLCPFGEVENATRFASVYAWNRKRISNCVADYSVLYNS
ASFSTFKCYGVSPTKLNDLCFTNVYADSFVIRGDEVRQIAPGQTGKIADYNYKLPDDETGCV
IAWNSNNLDSKVGGNYNYLYRLFRKSNLKPFERDISTEIYQAGSTPCNGVEGENCYFPLQSY
GFQPTNGVGYQPYRVVVLSFELLHAPATVCGPKKSTNLVKNKCVNFNFNGLTGTGVLTESNK
KFLPFQQFGRDIADTTDAVRDPQTLEILDITPCSFGGVSVITPGTNTSNQVAVLYQDVNCTE
VPVAIHADQLTPTWRVYSTGSNVFQTRAGCLIGAEHVNNSYECDIPIGAGICASYQTQTNSP
GSASSVASQSIIAYTMSLGAENSVAYSNNSIAIPTNFTISVTTEILPVSMTKTSVDCTMYIC
GDSTECSNLLLQYGSFCTQLNRALTGIAVEQDKNTQEVFAQVKQIYKTPPIKDFGGFNFSQI
LPDPSKPSKRSPIEDLLENKVTLADAGFIKQYGDCLGDIAARDLICAQKENGLTVLPPLLTD
EMIAQYTSALLAGTITSGWTFGAGPALQIPFPMQMAYRENGIGVTQNVLYENQKLIANQENS
AIGKIQDSLSSTPSALGKLQDVVNQNAQALNTLVKQLSSNFGAISSVLNDILSRLDPPEAEV
QIDRLITGRLQSLQTYVTQQLIRAAEIRASANLAATKMSECVLGQSKRVDFCGKGYHLMSFP
QSAPHGVVFLHVTYVPAQEKNFTTAPAICHDGKAHFPREGVFVSNGTHWFVTQRNFYEPQII
TTDNTFVSGNCDVVIGIVNNTVYDPLQPELDSGGDIIKLLNEQVNKEMQSSNLYMSMSSWCY
THSLDGAGLFLFDHAAEEYEHAKKLIIFLNENNVPVQLTSISAPEHKFEGLTQIFQKAYEHE
QHISESINNIVDHAIKSKDHATFNFLQWYVAEQHEEEVLENDILDKIELIGNENHGLYLADQ
YVKGIAKSRKS

It is understood that the examples and embodiments described in the present disclosure are for illustrative purposes only and that various modifications or changes in light thereof will be suggested to persons skilled in the art and are to be included within the spirit and purview of this application and scope of the appended claims. All publications, patents, and patent applications cited in the present disclosure are hereby incorporated by reference in their entirety for all purposes.

### PUBLICATIONS CITED IN THIS DISCLOSURE

Altschul et al., 1990, “Basic local alignment search tool.” J. Mol. Biol. 215: 403-410.
Altschul et al., 1977, “Gapped BLAST and PSI-BLAST: a new generation of protein database search programs” Nucleic Acids Res. 25: 3389-3402.
Amanat et al., 2020, “A serological assay to detect SARS-CoV-2 seroconversion in humans.” Nature Medicine 26:1033-1036.
Brouwer et al., 2020, “Potent neutralizing antibodies from COVID-19 patients define multiple targets of vulnerability.” Science 369(6504):643-650/
Chen et al., 2013, “Fusion protein linkers: Property, design and functionality.” Adv. Drug Deliv. Rev. 65(10): 1357-1369 (doi: 10.1016/j.addr.2012.9.039).
Crawford et al., 2020, “Protocol and Reagents for Pseudotyping Lentiviral Particles with SARS-CoV-2 Spike Protein for Neutralization Assays.” Viruses 12(5):513-542.
He et al., 2016, “Presenting native-like trimer HIV-1 antigens with self-assembling nanoparticles.” Nat. Commun. 7:12041 doi:10.1038/ncomms12041.
Henikoff and Henikoff, 1989, “Amino acid substitution matrices from protein blocks” Proc. Natl. Acad. Sci. USA 89:10915-10919.
Hsieh et al., 2020, “Structure-based design of prefusion-stabilized SARS-CoV-2 spikes.” Science 369:1501-1505.
Kanekiyo et al., 2013, “Self-assembling influenza nanoparticle vaccines elicit broadly neutralizing H1N1 antibodies.” Nature 499(7456):102-106 doi: 10.1038/nature12202.
Kanekiyo et al., 2015, “Rational design of an Epstein-Barr virus vaccine targeting the receptor-binding site.” Cell 162(5):1090-100 doi: 10.1016/j.cell.2015.07.043.
Karlin and Altschul, 1993, “Applications and statistics for multiple high-scoring segments in molecular sequences.” Proc. Nat'l. Acad. Sci. USA 90:5873-5787.
Kimple et al., 2013, “Overview of Affinity Tags for Protein Purification,” Curr. Protoc. Protein Sci. 73: Unit-9.9.
Low et al., 2013, “Optimisation of signal peptide for recombinant protein secretion in bacterial hosts.” Applied Microbiology and Biotechnology 97:3811-3826.
Needleman and Wunsch, 1970, “A general method applicable to the search for similarities in the amino acid sequence of two proteins.” J. Mol. Biol. 48:443-453.
Pearson and Lipman, 1988, “Improved tools for biological sequence comparison.” Proc. Natl. Acad. Sci. (U.S.A.) 85: 2444-2448.
Rohou et al., 2015, “CTFFIND4: Fast and accurate defocus estimation from electron micrographs.” J. Struct. Biol. 192(2):216-221.
Rogers et al., 2020, “Isolation of potent SARS-CoV-2 neutralizing antibodies and protection from disease in a small animal model.” Science Jun. 15, 2020 doi: 10.1126/science.abc7520
Smith and Waterman, 1981, “Comparison of biosequences.” Add. APL. Math. 2:482-489.
Wu et al., 2020, “A new coronavirus associated with human respiratory disease in China.” Nature 579:265-269.
Walls et al., 2020, “Structure, function and antigenicity of the SARS-CoV-2 spike glycoprotein.” Cell 181:281-292.
Wrapp et al., 2020, “Cryo-EM structure of the 2019-nCoV spike in the prefusion conformation.” Science 367(6483):1260-1263.
Zhang, 2011, “Self-assembly in the ferritin nano-cage protein super family.” Int. J. Mol. Sci. 12:5406-5421.
Zhang et al., 2020, Probable Pangolin Origin of SARS-CoV-2 Associated with the COVID-19 Outbreak.” Current Biology 30:1346-1351

What is claimed is:

1. A fusion protein of an artificially modified amino acid sequence of a Spike protein of a coronavirus and an amino acid sequence of a ferritin subunit polypeptide, wherein the artificially modified amino acid sequence of the Spike protein is a sequence with at least 90% sequence identity to SEQ ID NO:3, SEQ ID NO:4, SEQ ID NO:14, or SEQ ID NO:15.

2. The fusion protein of claim 1, wherein the coronavirus is SARS-CoV-2.

3. The fusion protein of claim 1 or 2, wherein the artificially modified amino acid sequence of the Spike protein of the coronavirus comprises a C-terminal deletion of at least an amino acid sequence of heptad repeat 2 (H1R2).

4. The fusion protein of any one of claims 1 to 3, wherein the artificially modified amino acid sequence of the Spike protein of the coronavirus comprises a mutation eliminating a furin recognition site.

5. The fusion protein of any one of claims 1 to 4, wherein the artificially modified amino acid sequence of the Spike protein of the coronavirus comprises one or more mutations stabilizing the Spike protein a pre-fusion conformation.

6. The fusion protein of any one of claims 1 to 5, wherein the ferritin subunit polypeptide is Helicobacter pylori ferritin subunit polypeptide.

7. The fusion protein of any one of claims 1 to 6, wherein the amino acid sequence of the ferritin subunit polypeptide is a sequence with at least 90% sequence identity to SEQ ID NO:2.

8. The fusion protein of any one of claims 1 to 7, wherein the ferritin subunit polypeptide contains one or more artificial glycosylation sites.

9. The fusion protein of any one of claims 1 to 8, wherein the artificially modified amino acid sequence of the Spike protein of the coronavirus is joined to the amino acid sequence of the ferritin subunit polypeptide by a linker amino acid sequence.

10. The fusion protein of any one of claims 1 to 9, wherein the amino acid sequence of the fusion protein is a sequence with at least 90% sequence identity to SEQ ID NO:12, SEQ ID NO:13, SEQ ID NO:16, SEQ ID NO:17, SEQ ID NO:18, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:33, or SEQ ID NO:34.

11. A nanoparticle comprising an oligomer of the fusion protein of any one of claims 1-10.

12. The nanoparticle of claim 11, wherein the nanoparticle comprises surface-exposed trimers of an ectodomain of the Spike protein of the coronavirus.

13. The nanoparticle of claim 12, wherein the nanoparticle comprises eight of the surface-exposed trimers of the ectodomain of the Spike protein of the coronavirus.

14. A nucleic acid encoding the fusion protein of any one of claims 1 to 10.

15. The nucleic acid of claim 14, wherein the nucleic acid is DNA or RNA.

16. A vector comprising the nucleic acid of claim 14 or 15.

17. A cell comprising the nucleic acid of claim 14 or 15 or the vector of claim 14.

18. An immunogenic composition comprising the fusion protein of any one of claims 1-9, the nanoparticle of any one of claims 10 to 12, the nucleic acid of claim 13 or 14, or the vector of claim 15.

19. An immunogenic composition comprising two or more different fusion proteins of any one of claims 1 to 9, two or more different nanoparticles of any one of claims 10 to 12, two or more different nucleic acids of claim 13 or 14, or two or more different vectors of claim 15.

20. The immunogenic composition of claim 18 or 19, further comprising one or more adjuvants.

21. The immunogenic composition of any one of claims 18 to 20, wherein the one or more adjuvants comprise alum.

22. The immunogenic composition of claim 18 or 20, wherein the immunogenic composition is lyophilized.

23. A kit comprising the immunogenic composition of any one of claims 18 to 22 and one or more of: a device for administering the immunogenic composition, and an excipient.

24. A method of inducing an immune response in a subject, the method comprising administering to the subject the immunogenic composition of any one of claims 18 to 22.

25. The method of claim 24, wherein the immunogenic composition is administered in an amount capable of eliciting a protective immune response against the coronavirus in the subject.

26. The method of claim 25, wherein the protective immune response comprises production of neutralizing antibodies against the coronavirus in the subject.

27. The method of any one of claim 24 to 26, wherein the subject is a human.

28. A method of producing the fusion protein, comprising:
introducing into a cell the nucleic acid of claim 14 or 15 or the vector of claim 15;
incubating the cell under conditions allowing for expression of the fusion protein; and,
isolating the fusion protein.

29. A method of producing a nanoparticle, comprising:
introducing into a cell the nucleic acid of claim 14 or 15 or the vector of claim 15;
incubating the cell under conditions allowing for expression of the fusion protein and self-assembly of the nanoparticle; and,
isolating the nanoparticle.
